(19) United States
(12) Patent Application Publication
(10) Pub. No.: US 2023/0374540 A1
(43) Pub. Date: Nov. 23, 2023

(54) IMPROVED ADENO-ASSOCIATED VIRUS GENE THERAPY VECTORS

(71) Applicant: FERRING VENTURES SA, Lausanne (CH)

(72) Inventors: Lionel GALIBERT, Lausanne (CH); Kari AIRENNE, Lausanne (CH); Amira HYVÖNEN, Lausanne (CH); Reetta ERIKSSON, Lausanne (CH); Hanna Lesch, Lausanne (CH); Justin Darius ALBERS, Lausanne (CH)

(21) Appl. No.: 18/002,585

(22) PCT Filed: Jun. 25, 2021
(86) PCT No.: PCT/EP2021/067569

§ 371 Date: Jul. 25, 2023

Related U.S. Application Data
(60) Provisional application No. 63/043,837, filed on Jun. 25, 2020.

(51) Int. Cl. C12N 15/86; C07K 14/015

(52) U.S. Cl. CPC C12N 15/86; C07K 14/015; C12N 2750/14122; C12N 2750/14143; C12N 2750/14151

(57) ABSTRACT

“MAAP” is a naturally-occurring, newly-discovered about 13 KDa adeno-associated vims protein. It is not homologous to known proteins. When AAV producer cells are cultured for more than 24 hours, we found that inactivating translation of the full-length MAAP improves the productivity of the transfected producer cells. The resulting AAV viruses are also of better quality and more stable. Our findings thus provide a way to improve the industrial manufacture of recombinant adeno-associated virus gene therapy vectors.

### CROSS-REFERENCE TO RELATED APPLICATIONS

This patent application claims the benefit of U.S. Prov. Ser. No. 63/043,837 filed Jun. 25, 2020, which is incorporated by reference herein.

### BACKGROUND

Adeno-associated virus (AAV) is a dependo-parvovirus. Viral replication depends on the infected cell being co-infected with a helper virus such as Adenovirus or Herpesvirus.

AAV is highly prevalent in humans and other primates. Several serotypes have been isolated from various tissue samples.

More than 12 natural serotypes and over 100 variants of AAV have been isolated, studied and applied as gene delivery vectors, and new variants are continuously been generated to improve AAV for gene delivery. Among the most studied AAV are serotypes 2, 3, 5, 6, 9 and 12, discovered in human cells, and serotypes 1, 4, 7, 8, 10 and 11, discovered in non-human primate cells. The International Committee on Taxonomy of Viruses parses these various serotypes into two broad species: A and B. The A species encompasses e.g., serotypes -1, -2, -3 and -4, while the B species encompasses serotype -5. In addition to primates, AAVs have also been isolated from other species such as horse, cow, chicken, snake, lizard, and goat.

Each serotype has some degree of tissue specificity. For example, serotype 6 is efficient at infecting human heart cells, while serotype 8 is efficient at infecting human liver and skeletal muscle cells.

Serotype, and thus tissue specificity, is determined by the capsid. AAV capsid proteins contain 12 hyper-variable surface regions. Most variability occurs in the three-fold proximal peaks.

The genomes of all known serotypes share a similar organization. Serotype 2 (AAV2), for example, has a genome of 4679 bases. The AAV2 genome is flanked at both ends by 145-base T-shaped structures, Inverted Terminal Repeats (ITRs). ITRs are necessary for genome replication, second-strand synthesis, encapsidation and insertion of the viral genome into the human genome. In AAV2, genome replication is mediated by two large rep proteins, Rep78 and Rep68. The small rep proteins, Rep52 and Rep40, are required for the packaging of either the positive or the negative strand of the AAV genome in preformed empty capsids.

The cap gene expresses the 3 capsid proteins, VP1, VP2 and VP3. It does so through alternative splicing, the use of non-ATG start codons, overlapping reading frames. Assembly Activating Protein (AAP), expressed through a frame-shift in VP2/3 reading frame, targets the VP proteins to the nucleolus. This is required for capsid assembly.

The wild-type AAV capsid is icosahedral. It is composed of 60 VP protein molecules. The wild-type capsid displays a VP1:VP2:VP3 ratio of 1:1:10. VP3 thus commonly forms the “core” of the capsid.

The intra-cellular compartmentalization of AAV, studied in the context of wild-type Adenovirus and wild-type AAV (wt-AAV) co-infection in Hela cells, suggests the nucleolus to be involved in the initiation of capsid assembly while the DNA packaging occurs in the nucleoplasm. At a later stage, the Rep proteins are enriched at the nuclear periphery. Assembled AAV capsids can be observed co-localizing with AAP either in the nucleus, the nucleolus, or clustered around the nuclear membrane.

AAV is known as an attractive potential gene therapy vector. Full therapeutic employment of AAV, however, faces several hurdles. For example, in a commercial manufacturing setting in vitro it is preferred to culture infected and/or transfected producer cells for at least 72 hours or more, to increase the amount of virus produced. AAV, however, can degrade rapidly during manufacture. For example, while it is preferred to culture infected producer cells for at least 72 hours to assure a prolific harvest of virus, serotypes 2 and 8 degrade in less than 72 hours, thus reducing the final yield of infective virus.

It is possible to overcome this by harvesting the virus from producer cells only 24 hours after infection. This reduces the degradation of the completed virus, but in prematurely halting virus replication, it also reduces the amount of virus produced.

Hence, there remains a need in the art to provide methods wherein high quality and stable AAV, in particular AAV for gene therapy, can be prepared in sufficient amounts.

### BRIEF SUMMARY

The disclosure provides the following preferred embodiments. However, the invention is not limited to these embodiments.

In one aspect, the disclosure provides an adeno-associated virus genome that has a mutation that inactivates the membrane-associated accessory protein (MAAP) mRNA translation-initiation codon or introduces at least one stop codon to stop translation of full-length wild-type MAAP.

In a further aspect, the disclosure provides an adeno-associated virus genome that has a mutation that reduces expression of full-length wild-type MAAP.

In preferred embodiments, expression of VP1 is maintained.

In a further aspect, the disclosure provides an adeno-associated virus genome that transcribes to MAAP mRNA, the genome having a mutation whereby the MAAP mRNA is altered from wild-type MAAP mRNA, the alteration selected from: changing the MAAP translation initiation codon to a sequence that is not an initiation codon, and creating at least one stop codon in the MAAP mRNA; wherein said mutation does not prevent expression of VP1 from said genome.

In preferred embodiments, said mutation inactivates the MAAP translation initiation codon and/or introduces at least one stop codon to stop translation of full-length wild-type MAAP.

In preferred embodiments, said mutation inactivates the MAAP translation initiation codon.

In preferred embodiments, said mutation introduces at least one stop codon to stop translation of MAAP polypeptide at a polypeptide residue aligning with polypeptide consensus sequence SEQ ID NO. 11 residue number 9, 33, 39, 47, 65, 90, 100, 103, 105, 106 or 110.

In preferred embodiments, said mutation introduces at least one stop codon to stop translation of MAAP polypeptide at a polypeptide residue aligning with polypeptide consensus sequence SEQ ID NO. 11 from residue numbers 9 to 110, more preferably from residue numbers 39 to 103.

In preferred embodiments, said mutation introduces at least one stop codon to stop translation of MAAP polypeptide at a polypeptide residue aligning with MAAP polypeptide consensus sequence SEQ ID NO. 11 residue number 9, 33, 39, 47, 65, 90, 100, 106 or 110.

In preferred embodiments, said mutation introduces a stop codon to stop translation of MAAP polypeptide at a polypeptide residue aligning with MAAP polypeptide consensus sequence SEQ ID NO. 11 residue number 9, 33, 39 and/or 47.

In preferred embodiments, said mutation introduces a stop codon to stop translation of MAAP polypeptide at a polypeptide residue aligning with AP polypeptide consensus sequence SEQ ID NO. 11 residue number 9 or residue numbers 33, 39 and 47.

In preferred embodiments, the genome is selected from the group consisting of: a naturally-occurring serotype and a non-naturally-occurring serotype.

In preferred embodiments, the genome is selected from a serotype 1 genome, a serotype 2 genome, a serotype 5 genome, a serotype 6 genome, a serotype 8 genome, a serotype 9 genome, a serotype 10 genome and a non-naturally-occurring serotype.

In preferred embodiments, the genome is selected from a serotype 1 genome, serotype 2 genome, serotype 6 genome, serotype 7 genome, serotype 8 genome and a serotype 10 genome.

In preferred embodiments, the genome comprises a serotype 1, 2, 5, 6, 8 or 9 genome.

In preferred embodiments, the genome comprises a serotype 2, 5, 6 or 8 genome.

In preferred embodiments, the genome comprises a serotype 2 genome.

In preferred embodiments, the genome comprises a non-naturally-occurring serotype.

In preferred embodiments, the VP1 peptide sequence is unaltered from wild type.

In preferred embodiments, the VP1 peptide sequence contains a mutation, such as a conservative mutation. Typically, the VP1 peptide is altered at the location(s) corresponding to where the MAAP peptide sequence has been mutated to include a stop codon.

In preferred embodiments, the MAAP and VP1 peptide sequences each have at least 80% homology to wild type.

In preferred embodiments, the MAAP and VP1 peptide sequences each have at least 90% homology to wild type.

In a further aspect is provided an adeno-associated virus genome that does not express a polypeptide having a primary amino acid sequence having at least 50% homology to any 33 contiguous residues of MAAP consensus polypeptide sequence SEQ. ID NO. 11, i.e. a viral particle, vector or plasmid comprising the AAV genome does not express such polypeptide when present in a suitable host cell, i.e. a host cell that allows expression of proteins encoded by the AAV genome. In preferred embodiments, the AAV genome does not express a polypeptide having a primary amino acid sequence having at least 50% identity to any 33 contiguous residues of MAAP consensus polypeptide sequence SEQ. ID NO. 11.

In a further aspect, the disclosure provides a producer cell that produces adeno-associated virus, the producer cell comprising an adeno-associated virus genome of the invention.

In preferred embodiments, the producer cell is eukaryotic.

In preferred embodiments, the producer cell comprises a human cell.

In preferred embodiments, the producer cell is selected from the group consisting of yeast cells and insect cells.

In a further aspect, the disclosure provides a method for producing adeno-associated virus, the method comprising: obtaining an adeno-associated virus genome, and then introducing said genome into a cell to create a producer cell of the invention, and then culturing said producer cell whereby said producer cell produces adeno-associated virus, and then harvesting said adeno-associated virus.

In preferred embodiments, said harvested adeno-associated virus comprises a transgene.

In preferred embodiments, the producer cell produces virus preparation wherein the ratio of the number of capsids containing the gene or genome of interest to the number of total physical capsids is at least as high as the ratio of the number of capsids containing the gene or genome of interest to the number of total physical capsids produced by a similar cell containing a wild-type adeno-associated virus genome.

In preferred embodiments, a producer cell of the invention produces virus having a ratio of full: empty virus capsids least as high as does a similar cell infected with a wild-type adeno-associated virus genome.

In preferred embodiments, the producer cell produces virus having at least as many viral genomes/mL as does a similar cell infected with wild-type adeno-associated virus.

In preferred embodiments, the producer cell produces virus having at least four times as many viral genomes/mL as does a similar cell infected with wild-type adeno-associated virus.

In preferred embodiments, the producer cell produces virus having a ratio of full: empty virus capsids 30% higher than does a similar cell infected with wild-type adeno-associated virus.

In a further aspect, the disclosure provides an adeno-associated virus genome that has a mutation that inactivates the MAAP mRNA translation-initiation codon.

In preferred embodiments, said adeno-associated virus genome further comprises at least one mutation that introduces at least one stop codon to stop translation of full-length wild-type MAAP.

In preferred embodiments, said mutation introduces at least one stop codon to stop translation of MAAP polypeptide at a polypeptide residue aligning with MAAP polypeptide consensus sequence SEQ ID NO. 11 residue number 9, 33, 39, 47, 65, 90, 100, 103, 105, 106 or 110.

In preferred embodiments, wherein said mutation introduces stop codons to stop translation of MAAP polypeptide at a polypeptide residue aligning with polypeptide consensus sequence SEQ ID NO. 11 residue numbers 33, 39 and 47.

In preferred embodiments, the genome is selected from the group consisting of: adeno-associated virus a serotype 1, serotype 2, serotype 3, serotype 4, serotype 5, serotype 6, serotype 7, serotype 8, serotype 9, serotype 10 and non-naturally-occurring serotype.

In preferred embodiments, the genome comprises a serotype 2 genome.

In preferred embodiments, the genome comprises a non-naturally-occurring serotype.

In a further aspect, the disclosure provides an adeno-associated virus genome that does not express a polypeptide having a primary amino acid sequence having at least 50% homology to any 33 contiguous residues of MAAP consensus polypeptide sequence SEQ. ID NO. 11.

In preferred embodiments, the 33 contiguous residues comprise MAAP consensus polypeptide sequence SEQ. ID NO. 11 residues 93 to 97.

In preferred embodiments, the 33 contiguous residues comprise MAAP consensus polypeptide sequence SEQ. ID NO. 11 residues 107 to 119.

In preferred embodiments, the 33 contiguous residues comprise MAAP consensus polypeptide sequence SEQ. ID NO. 11 residues 1 to 30.

In preferred embodiments, the adeno-associated virus genome is free of any sequence having at least 60% homology to MAAP consensus polypeptide sequence SEQ. ID NO. 11 residues 1 to 33.

In preferred embodiments, the adeno-associated virus genome is free of any sequence having at least 60% homology to MAAP consensus polypeptide sequence SEQ. ID NO. 11 residues 1 to 39 or residues 1 to 47.

In preferred embodiments, the adeno-associated virus genome is free of any sequence having at least 60% homology to any 30 contiguous residues of MAAP consensus polypeptide sequence SEQ. ID NO. 11.

In preferred embodiments, the adeno-associated virus genome is free of any sequence having at least 70% homology to any 30 contiguous residues of MAAP consensus polypeptide sequence SEQ. ID NO. 11.

In preferred embodiments, the adeno-associated virus genome is free of any sequence having at least 80% homology to any 30 contiguous residues of MAAP consensus polypeptide sequence SEQ. ID NO. 11.

In a further aspect, the disclosure provides an adeno-associated virus genome that does not express a polypeptide having a primary amino acid sequence having at least 95% homology to any 15 contiguous residues of MAAP consensus polypeptide sequence SEQ. ID NO. 11.

In preferred embodiments, the genome does not express a polypeptide having a primary amino acid sequence having at least 90% homology to any 17, preferably 19, preferably 21 contiguous residues of MAAP consensus polypeptide sequence SEQ. ID NO. 11.

In a further aspect, the disclosure provides an adeno-associated virus genome that does not express a polypeptide having a primary amino acid sequence having at least 50% homology to any 10 contiguous residues of MAAP consensus polypeptide sequence SEQ. ID NO. 11 residue numbers 94 to 120.

In a further aspect, the disclosure provides a method for producing adeno-associated virus, introducing into a cell an adeno-associated virus genome of the invention to make a producer cell, and then culturing said producer cell to make adeno-associated virus, and then harvesting said adeno-associated virus.

In a further aspect, the disclosure provides a method for producing adeno-associated virus, the method comprising: inserting an adeno-associated virus genome of the invention into a cell to make a producer cell, and then culturing said producer cell to make adeno-associated virus, and then harvesting said adeno-associated virus.

In a further aspect, the disclosure provides a producer cell that produces adeno-associated virus, the producer cell substantially free of polypeptide having at least 50% homology to any 30 contiguous residues of consensus polypeptide sequence SEQ. ID NO. 11.

In a further aspect, the disclosure provides a producer cell that produces adeno-associated virus, the producer cell substantially free of polypeptide having at least 95% homology to any 15 contiguous residues of M consensus polypeptide sequence SEQ. ID NO. 11.

In a further aspect, the disclosure provides a producer cell that produces adeno-associated virus, the producer cell substantially free of polypeptide having at least 50% homology to any 10 contiguous residues of MAAP consensus polypeptide sequence SEQ. ID NO. 11 residue numbers 94 to 120.

In a further aspect, the disclosure provides a producer cell comprising an adeno-associated virus genome, the producer cell able to express adeno-associated virus, the producer cell substantially free of full-length functional MAAP.

In preferred embodiments, the producer cell is eukaryotic.

In preferred embodiments, the producer cell comprises a human cell.

In preferred embodiments, the producer cell is selected from the group consisting of yeast cells and insect cells.

In preferred embodiments, said adeno-associated virus genome has a mutation that interferes with the expression of full-length, wild-type functional MAAP.

In preferred embodiments, said producer cell comprises a protein, such as a monoclonal antibody or affibody, directed against MAAP that binds to MAAP and impairs the function of MAAP.

In a further aspect the disclosure provides a producer cell comprising an adeno-associated virus genome, the producer cell able to express adeno-associated virus, the producer cell substantially free of full-length functional MAAP. In preferred embodiments, the adeno-associated virus genome has a mutation that interferes with the expression of full-length, wild-type functional MAAP. In preferred embodiments, the producer cell comprises interfering RNA that interferes with the expression of full-length, wild-type functional MAAP. In preferred embodiments, comprises a monoclonal antibody directed against MAAP that binds to MAAP and impairs the function of MAAP.

In a further aspect, the disclosure provides a method for producing adeno-associated virus, the method comprising culturing a producer cell of the invention whereby the producer cell produces adeno-associated virus, and then harvesting said adeno-associated virus.

In a further aspect, the disclosure provides an adeno-associated virus produced by a process of the invention.

In a further aspect, the disclosure provides a method of increasing stability, increasing capsid integrity, or reducing capsid degradation of an adeno-associated virus (AAV), comprising including in the AAV the adeno-associated virus genome of the invention.

In a further aspect, the disclosure provides a method of increasing the proportion of AAV capsids containing a gene or genome of interest, comprising including in the AAV the adeno-associated virus genome of the invention and the gene or genome of interest.

In a further aspect, the disclosure provides a method of the increasing the viral titre (viral genomes/mL) of a producer cell producing an AAV, comprising including in the AAV the adeno-associated virus genome of the invention and introducing the AAV in the producer cell.

In preferred embodiments, the producer cell is cultured for at least 30 hours. In preferred embodiments, the producer cell is cultured for at least 36 hours, 48 hours, 72 hours or 96 hours.

In a further aspect, the disclosure provides a method for increasing the retention of viral genomes or viral particles in a producer cell producing an AAV, comprising including in the AAV the adeno-associated virus genome of any one of claims 1-47 and introducing the AAV in the producer cell.

In preferred embodiments, the method further comprising harvesting and/or purifying the viral genomes or viral particles from the producer cells, preferably substantially free of media.

### DETAILED DESCRIPTION

Despite the success of recombinant adeno-associated virus (rAAV) for gene therapy treatments, their availability is limited due the limitations in large-scale manufacturing. Variants of the recently identified ORF encoding the membrane-associated accessory protein (MAAP) that is encoded by the cap gene in the same genomic region as the VP1/2 unique domain, may help address productivity limitations for most AAV serotypes. It is shown herein that some C-terminally truncated MAAP variants lead to improved wild type AAV2 productivity and reduced capsid degradation, without affecting the VP amino acid sequence. Further, two structurally diverse examples of MAAP variants were used for the production of rAAV serotypes 1, 2, 5, 6, 8 and 9 encoding the murine Secreted Alkaline Phosphatase (mSeAP) gene. As shown in Example 2, the MAAP variants generally led to an increase in rAAV production yields and an increase in the percentage of capsids containing the rAAV genome. The presence of the vector in the cell or in the media was altered for some AAV serotypes. Based on cap gene sequences of several AAV serotypes, a MAAP phylogenetic tree was constructed, which connects certain biological properties with the major clades of MAAP. This phylogenetic tool allows estimates of the potential productivity gains and distribution of the vector in the cell and in the media for their particular capsid variants when using specific MAAP variants, but also reasonably predicts the consistency of many of these properties across AAV serotypes.

We serendipitously found a way to both increase production while also making a better-quality vector. Using our new approach, one can increase viral production 72 hours after infection by 300-400%. The resulting viruses are more stable, showing less capsid degradation ≥72 hours after infection. The resulting virus, if designed to be a gene therapy vector (i.e., if it is recombinant and includes a “transgene” or therapeutic foreign gene), shows also improved genome packaging at 72 h. Furthermore, the resulting gene therapy vector is expected to achieve improved transduction efficiency (expression of the therapeutic transgene in the target cells).

While our discovery is important industrially, we came across it while doing research more academic or theoretical in nature. Doing a virion characterization and whole-genome analysis of AAV, we identified a novel protein encoded by a non-canonical start codon and then discovered that the protein in fact is expressed in wild-type AAV. We named the novel protein “DS”. While carrying out our studies the same protein was described by Ogden et. al. (2019) who named it as a membrane-associated accessory protein (MAAP). For consistency, we also use the name MAAP herein instead of DS.

We found that mutation of the non-canonical start codon leads to inactivation of this new protein. We similarly found that the introduction of multiple stop codons in the N-terminus of the accompanying ORF also leads to protein inactivation.

We then compared AAVs that were modified to include stop codons to inactivate this new protein to wild-type AAV. We found that 24 h after infection, wt-AAV produces higher viral titers than do viruses modified to include stop codons to inactivate the new protein. This is perhaps not surprising, because it implies that the wild-type gene gives some kind of selective advantage viz mutants lacking a working copy of that gene.

Surprisingly, however, we found that when we extended the period of culturing infected cells to 72 h, our mutant null viruses produced higher viral genome (vg) titers than did wt-AAV2. This is surprising because it implies that a wild-type gene has a deleterious selective disadvantage viz mutants lacking a working copy of that gene.

Further, we were surprised to find that our mutant (null) viruses show more robust capsid integrity or durability, and reduced capsid degradation over time. In contrast, wt-AAV displayed specific proteolytic fragments that are visible in immunoblotting.

Further, we found that our mutant (null) viruses show increased relative concentrations of VP1 and VP2 in the resulting capsids.

Further, we expect that our mutant (null) viruses are more infective than are wild-type AAV. Without intending to be bound by theory, we posit that mutant virus will be more infective due to higher capsid integrity, notably due to the VP1 protein. The VP1 unique domain encodes a phospholipase A2 domain, “PLA2.” This phospholipase is critical for AAV endosomal escape during the infection.

Similarly, we expect that our mutant (null) viruses, when engineered to contain a transgene, produce improved transgene expression. Without intending to be bound by theory, we posit that this improved transgene expression is because VP1 delivers the transgene to the infected cell nucleus for expression and our mutant (null) viruses have an increased relative concentration of VP1.

Although we do not wish to be bound theory, the MAAP C-terminus contains three basic-amino-acid-rich (BR) clusters, KKIR (MAAP2BR1), RRKR (MAAP2BR2) and RNLLRRLREKRGR (MAAP2BR3) that could be involved in the cellular localisation of MAAP. Indeed, similar BR clusters were shown to act as nuclear localisation signal (NLS) for AAP. The nearly complete deletion of the MAAPBR3 did not provide evidence of impaired nuclear localisation, so it remains that the MAAP2BR1 and MAAP2BR2 domains may still have allowed for membrane association. The lack of only the last ten amino acids at the MAAP C-terminus boosted VP and capsid levels and reduced capsid degradation while the total deletion of MAAP2BR3 fully prevented the degradation of AAV2 capsid. Proteasome inhibition plays a role during AAV infection and the addition of protease inhibitor can prevent capsid antigen presentation, as well enhance viral transduction. Furthermore, AAV capsid has been shown to be able to auto-cleave in acidic conditions, and AAV capsids are subjected to proteasome-involved post-translational modifications (PTMs) during wt-AAV production, including ubiquitination. These PTMs could potentially be used as signals to initiate host cell defence or to down-regulate new capsids via ubiquitination and subsequent proteasomal degradation. In addition to a possible impact of MAAP on the cellular degradation processes, the AAV stabilising effect of MAAP could be achieved by protecting the capsids from entering into subcellular localisations where the degradation process takes place. However, we did not observe an effect of MAAP on capsid egress from the nucleus within 24 hpt. Accordingly, in certain embodiments, MAAP peptides of the invention exclude, partially or fully, one or more of the BR clusters, particularly MAAP2BR3.

We thus found a surprising way to increase the industrial production of AAV vectors by inactivating expression of this novel wild-type protein. We also found a surprising way to increase the shelf-life stability of AAV viruses by inactivating expression of the wild-type protein. Our findings are industrially valuable because AAV can be therapeutically used as a gene therapy vector. Thus, our findings provide a way to make AAV gene therapy vectors (e.g., non-complimentary or self-complementary AAVs, AAVs with engineered ITRs, etc.) in larger quantity and with greater stability than previously possible.

By inactivating expression of the wild-type protein, one can increase viral production 72 hours after infection by 300-400%. The resulting viruses are more stable, showing less capsid degradation ≥72 hours after infection. The resulting virus has as high a percentage of full capsids as wild-type, and perhaps an even higher percentage of full capsids than wild type. Furthermore, we expect that the resulting gene therapy vector will achieve improved transduction efficiency (expression of the therapeutic transgene in the target cells).

As used herein, “to comprise” and its conjugations is used in its non-limiting sense to mean that items following the word are included, but items not specifically mentioned are not excluded. In addition, the verb “to consist” may be replaced by “to consist essentially of” meaning that a compound or adjunct compound as defined herein may comprise additional component(s) than the ones specifically identified, said additional component(s) not altering the unique characteristic of the invention.

The articles “a” and “an” are used herein to refer to one or to more than one (i.e., to at least one) of the grammatical object of the article. By way of example, “an element” means one element or more than one element. For example, a claim requiring “a stop codon” reads on one stop codon and several stop codons.

The word “approximately” or “about” when used in association with a numerical value (approximately 10, about 10) preferably means that the value may be the given value of 10 more or less 10% of the value.

As used herein the term “comparable” in the context of a particular value and a reference value means that the particular value is identical to the reference value, or deviates (being either higher or lower) from the reference value by at most 10%.

As used herein “membrane-associated accessory protein” or “MAAP” refers to a AAVP MAAP protein of any AAV serotype. As used herein, “wild-type MAAP” refers to naturally occurring, AAV MAAP. SEQ ID NO's 1-10 (see also FIG. 20) provide the amino acid sequence for the full length wild type MAAP for AAV serotypes 1-10 respectively. The amino acid sequence for each of these serotypes is highly conserved at the C-terminal end. At the N-terminal end, AAV serotype 4 (SEQ ID NO. 4) and serotype 5 (SEQ ID NO. 5) wild-type proteins have a leading 15-25 amino acid residue sequence not seen in the other serotypes. SEQ ID NO. 11 provides the primary amino acid sequence for the theoretical consensus of all ten of these serotypes. In preferred embodiments, a “wild-type MAAP” is a sequence selected from any of the sequences of SEQ ID NO's: 1 to 11. As used herein, “wild-type VP1” refers to naturally occurring AAV VP1.

The percentage of identity of an amino acid sequence or nucleic acid sequence, or the term “% sequence identity”, is defined herein as the percentage of residues of the full length of an amino acid sequence or nucleic acid sequence that is identical with the residues in a reference amino acid sequence or nucleic acid sequence after aligning the two sequences and introducing gaps, if necessary, to achieve the maximum percent identity. The percentage homology of an amino acid sequence or the term “% homology to” is defined herein as the percentage of amino acid residues in a particular sequence that are homologous with the amino acid residues in a reference sequence, after aligning the sequences and introducing gaps, if necessary, to achieve the maximum percent sequence homology. This method takes into account conservative amino acid substitutions. Conservative substitutions are substitutions of an amino acid to be substituted with a similar amino acid. Amino acids can be similar in several characteristics, for example, size, shape, hydrophobicity, hydrophilicity, charge, isoelectric point, polarity, aromaticity, etc. Preferably, a conservative substitution is an exchange of one amino acid within a group for another amino acid within the same group, whereby the groups are the following: (1) alanine, valine, leucine, isoleucine, methionine, and phenylalanine: (2) histidine, arginine, lysine, glutamine, and asparagine; (3) aspartate and glutamate; (4) serine, threonine, alanine, tyrosine, phenylalanine, tryptophan, and cysteine; and (5) glycine, proline, and alanine. Methods and computer programs for the alignment are well known in the art, for example “Align 2”. Programs for determining nucleotide sequence identity are also well known in the art, for example, the BESTFIT, FASTA and GAP programs. These programs are readily utilized with the default parameters recommended by the manufacturer.

If reference is made, e.g. in a claim, to mutated MAAP, then the claimed mutant need not be the same length as the wild-type polypeptide, but must be long enough to distinguish it from other, non-MAAP AAV polypeptides. For example, serotype 5 MAAP is 145 amino acid residues long. A claim requiring “mutated” serotype 5 MAAP reads on polypeptides that are greater than or less than 145 amino acids long, as long as the polypeptide has sufficient homology to serotype 5 MAAP to distinguish it from other, non-MAAP proteins. In contrast, the claim would not read on a polypeptide that is so short that it is indistinguishable from a wild-type AAV polypeptide that is not MAAP. This is because the artisan would not consider such a short polypeptide within the ambit of the claim term “MAAP.”

The terms “protein” and “polypeptide” refer to compounds comprising amino acids joined via peptide bonds and are used interchangeably. A protein or polypeptide encoded by a gene is not limited to the amino acid sequence encoded by a gene, but may include post-translational modifications of one or more amino acids of the protein or polypeptide. Sequences of proteins and polypeptides are depicted herein from N-terminal to C-terminal, unless otherwise indicated. As used herein with respect to the amino acids sequence of a protein or polypeptide, the terms “N-terminal” and “C-terminal” refer to relative positions in the amino acid sequence of the protein or polypeptide toward the N-terminus and the C-terminus, respectively. “N-terminus” and “C-terminus” refer to the extreme amino and carboxyl ends of the polypeptide, respectively.

As used herein reference to a specific amino acid residue or residues preferably refers to the residue with the corresponding number in the sequence of the MAAP sequences of SEQ ID NO's 1 to 11. When reference is made herein to a residue or residues in SEQ ID NO: 11 or to a residue or residues aligning with MAAP polypeptide consensus sequence SEQ ID NO. 11 residue number, the residue or residues in one of the MAAP amino acids sequences of AAV serotypes 1 to 10 (as depicted in SEQ ID NO's 1 to 10 and FIG. 20) that correspond to the indicated residue or residues in the consensus sequence of SEQ ID NO:11 are also encompassed. Hence, as used herein the term “a mutation at a polypeptide residue aligning with MAAP polypeptide consensus sequence SEQ ID NO. 11 residue number X” is defined as a mutation in a MAAP polypeptide at a position corresponding to amino acid residue number X of the MAAP polypeptide consensus sequence SEQ ID NO. 11. This can be either the indicated residue number in the sequence of SEQ ID NO:11 or a corresponding residue number in any AAV MAAP, in particular a corresponding residue number in a MAAP sequence having a sequence of any of SEQ ID NO's 1-10. In particular the corresponding residue or residues in MAAP of AAV serotype 1, 2, 5, 6, 8 and 9, more in particular AAV serotype 1, 2, 5, 6 and 8, are encompassed. A skilled person is well capable of determining the residue in any MAAP or an MAAP having a sequence of any of SEQ ID NO's 1 to 10 (MAAP amino acid sequence of AAV 1-10, respectively) corresponding to a particular residue in SEQ ID NO:11, e.g. by performing an alignment of the MAAP sequence and the sequence of SEQ ID NO:11. For example, in one embodiment, a mutation introduces a stop codon to stop translation of MAAP polypeptide at a polypeptide residue aligning with MAAP polypeptide consensus sequence SEQ ID NO. 11 residue number 65. As is clear to a skilled person, this refers to a mutation in a MAAP polypeptide at a position corresponding to amino acid residue number 65 of the MAAP polypeptide consensus sequence SEQ ID NO. 11. As an example residue number 65 in SEQ ID NO: 11 corresponds to residue number 64 in SEQ ID NO: 2.

In a first aspect is provided an adeno-associated virus genome that has a mutation that inactivates the MAAP mRNA translation-initiation codon and/or introduces at least one stop codon to stop translation of full-length wild-type MAAP. Also provided is an adeno-associated virus genome that has a mutation that reduces expression of full-length wild-type MAAP. In preferred embodiments, expression of VP1 is maintained. In particular, the mutation maintains expression of VP1.

As used herein the term “adeno-associated virus genome” refers to a polynucleotide molecule comprising at least one polynucleotide sequence encoding AAV MAAP. In preferred embodiments, the AAV genome or AAV vector comprises at least a gene encoding MAAP, in particular having a mutation that reduces expression of full-length wild-type MAAP, inactivates the membrane-associated accessory protein (MAAP) mRNA translation-initiation codon and/or introduces at least one stop codon to stop translation of full-length wild-type MAAP. The AAV genome or AAV vector preferably further comprises one or more polynucleotides sequences encoding one or more further AAV genes. In particular the genes other than the gene encoding may be wild-type or containing one or more mutations. In preferred embodiments, the AAV genome comprises a polynucleotide molecule comprising a AAV polynucleotide sequence flanked at both ends by AAV Inverted Terminal Repeats (ITRs). In preferred embodiments the AAV genome is encompassed by an AAV expression vector or is an AAV expression vector. The AAV genome or AAV expression vector thus preferably comprises at least one AAV polynucleotide, either wild-type or containing one or more mutations, flanked by AAV ITRs, as long as it contains a mutation that reduces expression of full-length wild-type MAAP, inactivates the membrane-associated accessory protein (MAAP) mRNA translation-initiation codon and/or introduces at least one stop codon to stop translation of full-length wild-type MAAP. In preferred embodiments, the AAV genome of the invention comprises a polynucleotide molecule comprising a AAV polynucleotide sequence that allows the production of AAV when introduced into a suitable host cell. Alternatively, the AAV genome of the invention is combined with one or more further polynucleotide molecules comprising AAV polynucleotide sequences, such as an AAV helper construct comprising a polynucleotide sequence encoding AAV capsid proteins and other AAV helper functions, so that the combined AAV genome and one or more further polynucleotide molecules allows the production of AAV when introduced into a suitable host cell.

In preferred embodiments, the AAV genome or AAV vector comprises the polynucleotide sequence of all AAV genes, either wild-type or containing one or more mutations, having at least one mutation that inactivates the membrane-associated accessory protein (MAAP) mRNA translation-initiation codon or introduces at least one stop codon to stop translation of full-length wild-type MAAP. The AAV genome or AAV vector may further comprise one or more heterologous polynucleotide, i.e. a polynucleotide other than a wild-type AAV gene, such as a transgene. An example of a transgene is a therapeutic gene.

The AAV genome or AAV vector may be of any AAV serotype, either a naturally occurring serotype or a non-naturally-occurring serotype. The cap genes encoding the MAAP are well known for each AAV serotype and a skilled person is therefore well capable of preparing a mutant AAV genome as described herein of any AAV serotype MAAP. As demonstrated in the examples herein, mutants have been prepared for multiple AAV serotypes and for all tested serotypes at least one of the effects described herein (e.g. higher viral titers after culturing for more than 24 hours, reduced capsid degradation, higher capsid integrity and VP protein integrity) were observed. In preferred embodiments, the AAV genome or AAV vector is a serotype 1 genome, a serotype 2 genome, a serotype 5 genome, a serotype 6 genome, a serotype 8 genome, a serotype 9 genome or a non-naturally-occurring serotype AAV genome or AAV vector. In preferred embodiments, the AAV genome or AAV vector is a serotype 1, 2, 5, 6, 8 or 9 AAV genome or AAV vector. In preferred embodiments, the AAV genome or AAV vector is a serotype 2, 5, 6 or 8 AAV genome or AAV vector. In preferred embodiment, the genome or vector comprises or is a serotype 2 genome or vector. In other preferred embodiments, the genome or vector comprises a non-naturally-occurring serotype genome or vector.

As used herein “reduces expression of full-length wild-type MAAP” means that the expression level of full-length wild-type MAAP by a viral particle, vector or plasmid comprising the AAV genome of the invention in a suitable host cell is reduced as compared to the expression level of full-length wild-type MAAP by a viral particle, vector or plasmid comprising an AAV genome that is identical to AAV genome of the invention with the exception that it lacks said mutation in the same host cell. In preferred embodiments, the expression of full-length wild-type MAAP is reduced by at least about 10%, preferably at least about 15%, more preferably at least about 20%, more preferably at least about 25%, more preferably at least about 50%, more preferably at least about 75%, more preferably at least about 80%, more preferably at least about 85%, more preferably at least about 90%, most preferably at least about 95%.

A mutation in the AAV genome that reduces expression of full-length wild-type MAAP is preferably a mutation whereby the MAAP mRNA is altered as compared to wild-type MAAP mRNA. In particular, altered as compared to wild-type MAAP mRNA of the same AAV serotype.

In preferred embodiments, a mutation in the AAV genome is a mutation in the gene encoding MAAP, in particular a mutation as compared to wild-type AAV. The gene encoding MAAP may have one or more such mutations. In one preferred embodiment, the gene encoding MAAP has one mutation. In other preferred embodiments, the gene encoding MAAP has between 1 and 10 mutations, preferably between 1 and 5 mutations, such as 1, 2, 3, 4 or 5 mutations. Said mutation(s) can be any type of mutation that has the indicated effect, e.g. a substitution, addition or deletion of one or more nucleotides. In preferred embodiments, said mutation is a substitution of one or more nucleotides, more preferably a substitution of one or more nucleotides resulting in the introduction of one or more stop codons in the MAAP amino acid sequence.

Mutations can for instance be introduced by site-directed mutagenesis. Site-directed mutagenesis is well known in the art and can be used to introduce one or more point mutations, including a mutation according to the invention (including substitution, insertion or deletion) into a viral polynucleotide or genome. A skilled person is well capable of introducing a mutation according to the invention. FIG. 21A-J provides the nucleic acid sequences of cap genes encoding inter alia MAAP and VP1 for AAV serotypes 1-10, respectively. Suitable techniques for site-directed mutagenesis are described in Sambrook's et al. Molecular Cloning:A Laboratory Manual, second edition (Cold Spring Harbor Laboratory Press, Cold Spring Harbor, N.Y. (1989)), Ausubel et al. Current Protocols in Molecular Biology(Greene Publishing Associates (1992)) and Bachman et al. (J. Methods Enzymol. 2013; 529:241-248), the content of which references is incorporated herein by reference.

In preferred embodiments, said mutation inactivates the MAAP mRNA translation initiation codon or introduces at least one stop codon to stop translation of full-length wild-type MAAP. Also provided is therefore an adeno-associated virus genome that transcribes to MAAP mRNA, the genome having a mutation whereby the MAAP mRNA is altered from wild-type MAAP mRNA, the alteration selected from the group consisting of: changing the MAAP translation initiation codon to a sequence that is not an initiation codon, creating at least one stop codon in the MAAP mRNA, and a combination thereof. In preferred embodiments said mutation does not prevent expression of VP1 from said genome.

In preferred embodiments, the mutation inactivates the MAAP (mRNA) translation initiation codon, i.e. the MAAP translation initiation codon is changed to a sequence that is not an initiation codon. Said translation initiation codon is preferably a non-ATG initiation codon, more preferably a CTG initiation codon, such as the first CTG in the nucleic acid sequence encoding MAAP, which translates to a leucine in MAAP of AAV serotype 2 (L1 on the full-length protein of AAV serotype 2). For instance, the CTG initiation codon can be mutated to CGG, inactivating the codon as a potential start codon. However, a person skilled in the art is well capable of introducing other mutations that inactivate an initiation codon.

In preferred embodiments, the mutation introduces at least one stop codon to stop translation of full-length wild-type MAAP. Said stop codon can be introduced at any position where its introduction has the result that full-length wild-type MAAP is no longer translated and/or expressed. Multiple stop codons can be introduced. In preferred embodiments 1-5 stop codons are introduced. In preferred embodiments 1-3 stop codons are introduced. preferred embodiments 1 or 3 stop codons are introduced. In preferred embodiments, the mutation is a mutation that introduces at least one stop codon to stop translation of full-length wild-type MAAP but that does not prevent expression VP1, preferably a mutation that introduces at least one stop codon to stop translation of full-length wild-type MAAP but that does not introduce a mutation in the VP1 amino acid sequence. In preferred embodiments, said mutation introduces at least one stop codon to stop translation of MAAP polypeptide at a polypeptide residue aligning with MAAP polypeptide consensus sequence SEQ ID NO. 11 from residue numbers 9 to 110, i.e. at a polypeptide residue in the sequence of SEQ ID NO. 11 from residue numbers 9 to 110 or a corresponding residue numbers in any MAAP amino acid sequence, in particular corresponding residue number in any of the sequences of SEQ ID NO's 1-10. In preferred embodiments, the mutation introduces at least one stop codon at a polypeptide residue aligning with MAAP polypeptide consensus sequence SEQ ID NO. 11 residue number 9, 33, 39, 47, 65, 90, 100, 103, 105, 106 and/or 110. In preferred embodiments, said mutation introduces at least one stop codon to stop translation of MAAP polypeptide at a polypeptide residue aligning with MAAP polypeptide consensus sequence SEQ ID NO. 11 from residue numbers 39 to 103. A skilled person is well capable of identifying the relevant residue number or numbers in any of the AAV naturally or non-naturally occurring serotypes based on the indicated residue numbers in the consensus sequence of SEQ ID NO:11. In preferred embodiments, the mutation introduces at least one stop codon at a polypeptide residue aligning with MAAP polypeptide consensus sequence SEQ ID NO. 11 residue number 9, 33, 39, 47, 65, 90, 100, 106 and/or 110. In preferred embodiments, the mutation introduced at least one stop codon at a polypeptide residue aligning with MAAP polypeptide consensus sequence SEQ ID NO. 11 residue number 9, 33, 39 and/or 47.

In preferred embodiments, the mutation is a mutation selected from the mutations indicated in table 1, table 2, table 3, or a combination of any of these mutations. In tables 1, 2 and 3 the mutated sequence is depicted in the column “MAAP mutated”.

In preferred embodiments, expression of VP1 is not prevented. In other preferred embodiments, expression of VP1 is maintained.

As used herein “maintains expression of VP1” and “does not prevent expression of VP1” mean that a viral particle, vector or plasmid comprising the AAV genome of the invention in a suitable host cell expresses VP1. “Maintains expression of VP1” and “does not prevent expression of VP1” preferably mean that the expression level of VP1 by such viral particle, vector or plasmid comprising the AAV genome of the invention in a suitable host cell is at least about 25% of the expression level of VP1 by a viral particle, vector or plasmid comprising an AAV genome that is identical to AAV genome of the invention with the exception that it lacks said mutation in the same host cell, more preferably at least about 50%, more preferably at least about 75%, more preferably at least about 90%, more preferably at least about 100%. In preferred embodiments, the term “maintains expression of VP1” means that the expression of VP1 by a viral particle, vector or plasmid comprising the AAV genome of the invention having said mutation in a suitable host cell is comparable to the expression of VP1 by a viral particle, vector or plasmid comprising an AAV genome that is identical to AAV genome of the invention with the exception that it lacks said mutation and in the same host cell.

Maintaining expression of VP1 or not preventing expression of VP1 is for instance achieved by introducing a mutation that reduces expression of full-length wild-type MAAP, that inactivates the MAAP mRNA translation-initiation codon or that introduces at least one stop codon to stop translation of full-length wild-type MAAP, but that does not result in a mutation of the VP1 amino acid sequence. Alternatively, this is achieved by introducing a mutation that results in a mutation in the VP1 amino acid sequence that does not affect expression of full-length VP1. In particular, such mutation also does not affect functionality of VP1. In preferred embodiments, the mutation reduces expression of full-length wild-type MAAP, that inactivates the MAAP mRNA translation-initiation codon or that introduces at least one stop codon to stop translation of full-length wild-type MAAP does not introduce a stop codon in VP1. Hence, in preferred embodiments, the VP1 amino acid sequence is unaltered. In particular, the VP1 amino acids sequences is unaltered as compared to wild-type VP1 amino acids sequence of the same AAV serotype. However, the VP1 amino acid or peptide sequence may contain one or more mutations. For instance, the VP1 peptide is altered at the location(s) corresponding to where the MAAP peptide sequence has been mutated to include a stop codon. Hence, in some embodiments, the VP1 amino acid sequence has one or more mutations. In some embodiments the one or more mutations in the VP1 peptide are conservative mutations. A skilled person is well capable of identifying or selecting an appropriate conservative mutation. Examples of conservative amino acid mutations, unlikely to affect the function of a protein or peptide, include the following: alanine for serine, valine for isoleucine, aspartate for glutamate, threonine for serine, alanine for glycine, alanine for threonine, serine for asparagine, alanine for valine, serine for glycine, tyrosine for phenylalanine, alanine for proline, lysine for arginine, aspartate for asparagine, leucine for isoleucine, leucine for valine, alanine for glutamate, aspartate for glycine, and vice versa. Preferably, a conservative substitution is an exchange of one amino acid within a group for another amino acid within the same group, whereby the groups are the following: (1) alanine, valine, leucine, isoleucine, methionine, and phenylalanine: (2) histidine, arginine, lysine, glutamine, and asparagine; (3) aspartate and glutamate; (4) serine, threonine, alanine, tyrosine, phenylalanine, tryptophan, and cysteine; and (5) glycine, proline, and alanine.

In preferred embodiments, the VP1 amino acid sequence has at least 80% homology to wild-type VP1, in particular wild-type VP1 of the same AAV serotype. In preferred embodiments, the VP1 amino acid sequence has at least 90%, more preferably at least 95%, more preferably at least 98%, homology to wild-type VP1, in particular wild-type VP1 of the same AAV serotype.

In preferred embodiments, the adeno-associated virus genome does not express a polypeptide having a primary amino acid sequence having at least 50% homology to any 33 contiguous residues of MAAP consensus polypeptide sequence SEQ. ID NO. 11. In preferred embodiments, the AAV genome does not express a polypeptide having a primary amino acid sequence having at least 50% identity to any 33 contiguous residues of MAAP consensus polypeptide sequence SEQ. ID NO. 11. Also provided is an adeno-associated virus genome that does not express a polypeptide having a primary amino acid sequence having at least 50% homology to any 33 contiguous residues of MAAP consensus polypeptide sequence SEQ. ID NO. 11. In preferred embodiments, the AAV genome does not express a polypeptide having a primary amino acid sequence having at least 50% identity to any 33 contiguous residues of MAAP consensus polypeptide sequence SEQ. ID NO. 11.

In preferred embodiments, the 33 contiguous residues comprise MAAP consensus polypeptide sequence SEQ. ID NO. 11 residues 93 to 97.

In preferred embodiments, the 33 contiguous residues comprise MAAP consensus polypeptide sequence SEQ. ID NO. 11 residues 107 to 119.

In preferred embodiments, the 33 contiguous residues comprise MAAP consensus polypeptide sequence SEQ. ID NO. 11 residues 1 to 30.

In preferred embodiments, the AAV genome is free of any sequence having at least 60% homology to MAAP consensus polypeptide sequence SEQ. ID NO. 11 residues 1 to 33. In preferred embodiments, the AAV genome is free of any sequence having at least 60% identity to MAAP consensus polypeptide sequence SEQ. ID NO. 11 residues 1 to 33.

In preferred embodiments, the AAV genome is free of any sequence having at least 60% homology to MAAP consensus polypeptide sequence SEQ. ID NO. 11 residues 1 to 39. In preferred embodiments, the AAV genome is free of any sequence having at least 60% identity to MAAP consensus polypeptide sequence SEQ. ID NO. 11 residues 1 to 39.

In preferred embodiments, the AAV genome is free of any sequence having at least 60% homology to MAAP consensus polypeptide sequence SEQ. ID NO. 11 residues 1 to 47. In preferred embodiments, the AAV genome is free of any sequence having at least 60% identity to MAAP consensus polypeptide sequence SEQ. ID NO. 11 residues 1 to 47.

In preferred embodiments, the AAV genome is free of any sequence having at least 60% homology to any 30 contiguous residues of MAAP consensus polypeptide sequence SEQ. ID NO. 11. In preferred embodiments, the AAV genome is free of any sequence having at least 60% identity to any 30 contiguous residues of MAAP consensus polypeptide sequence SEQ. ID NO. 11.

In preferred embodiments, the AAV genome is free of any sequence having at least 70% homology to any 30 contiguous residues of MAAP consensus polypeptide sequence SEQ. ID NO. 11. In preferred embodiments, the AAV genome is free of any sequence having at least 70% identity to any 30 contiguous residues of MAAP consensus polypeptide sequence SEQ. ID NO. 11.

In preferred embodiments, the AAV genome is free of any sequence having at least 80% homology to any 30 contiguous residues of MAAP consensus polypeptide sequence SEQ. ID NO. 11. In preferred embodiments, the AAV genome is free of any sequence having at least 80% identity to any 30 contiguous residues of MAAP consensus polypeptide sequence SEQ. ID NO. 11.

In preferred embodiments, the AAV genome the genome does not express a polypeptide having a primary amino acid sequence having at least 95% homology to any 15 contiguous residues of MAAP consensus polypeptide sequence SEQ. ID NO. 11. In preferred embodiments, the AAV genome the genome does not express a polypeptide having a primary amino acid sequence having at least 95% identity to any 15 contiguous residues of MAAP consensus polypeptide sequence SEQ. ID NO. 11.

In preferred embodiments, the AAV genome does not express a polypeptide having a primary amino acid sequence having at least 90% homology to any 17 contiguous residues of MAAP consensus polypeptide sequence SEQ. ID NO. 11. In preferred embodiments, the AAV genome does not express a polypeptide having a primary amino acid sequence having at least 90% identity to any 17 contiguous residues of MAAP consensus polypeptide sequence SEQ. ID NO. 11.

In preferred embodiments, the AAV genome does not express a polypeptide having a primary amino acid sequence having at least 90% homology to any 19 contiguous residues of MAAP consensus polypeptide sequence SEQ. ID NO. 11. In preferred embodiments, the AAV genome does not express a polypeptide having a primary amino acid sequence having at least 90% identity to any 19 contiguous residues of MAAP consensus polypeptide sequence SEQ. ID NO. 11.

In preferred embodiments, the AAV genome does not express a polypeptide having a primary amino acid sequence having at least 90% homology to any 21 contiguous residues of MAAP consensus polypeptide sequence SEQ. ID NO. 11. In preferred embodiments, the AAV genome does not express a polypeptide having a primary amino acid sequence having at least 90% identity to any 21 contiguous residues of MAAP consensus polypeptide sequence SEQ. ID NO. 11.

In preferred embodiments, the AAV genome does not express a polypeptide having a primary amino acid sequence having at least 50% homology to any 10 contiguous residues of MAAP consensus polypeptide sequence SEQ. ID NO. 11 residue numbers 94 to 120. In preferred embodiments, the AAV genome does not express a polypeptide having a primary amino acid sequence having at least 50% identity to any 10 contiguous residues of MAAP consensus polypeptide sequence SEQ. ID NO. 11 residue numbers 94 to 120.

In preferred embodiments, the adeno-associated virus genome according to the invention increases viral production when introduced into a suitable producer cell. In particular, viral production is increased as compared to viral production by the same host cell in which an AAV genome is introduced that is identical to AAV genome of the invention with the exception that it lacks a mutation that reduces expression of full-length wild-type MAAP, that inactivates the membrane-associated accessory protein (MAAP) mRNA translation-initiation codon and/or introduces at least one stop codon to stop translation of full-length wild-type MAAP as described herein. Preferably, viral production is increased by at least 10%. In preferred embodiments, viral production is increased by at least 15%, at least more preferably at least about 20%, more preferably at least about 25%, more preferably at least about 50%. In further preferred embodiments, viral production is increased by at least about 60%, at least about 75%, more preferably at least about 80%, more preferably at least about 85%, more preferably at least about 90%, most preferably at least about 95%.

In preferred embodiments, the adeno-associated virus genome according to the invention increases infectivity of a viral particle comprising the genome. In particular, infectivity is increased as compared to infectivity of a viral particle comprising an AAV genome that is identical to AAV genome of the invention with the exception that it lacks a mutation that reduces expression of full-length wild-type MAAP, that inactivates the membrane-associated accessory protein (MAAP) mRNA translation-initiation codon and/or introduces at least one stop codon to stop translation of full-length wild-type MAAP as described herein. Preferably, infectivity is increased by at least 10%. In preferred embodiments, infectivity is increased by at least 15%, at least more preferably at least about 20%, more preferably at least about 25%, more preferably at least about 50%. In further preferred embodiments, infectivity is increased by at least about 60%, at least about 75%, more preferably at least about 80%, more preferably at least about 85%, more preferably at least about 90%, most preferably at least about 95%.

In preferred embodiments, the adeno-associated virus genome according to the invention increases stability of a viral particle comprising the genome. In particular, the adeno-associated virus genome according to the invention reduces capsid degradation, increases capsid integrity and/or increases VP1 protein integrity. In particular, stability is increased as compared to stability of a viral particle comprising an AAV genome that is identical to AAV genome of the invention with the exception that it lacks a mutation that reduces expression of full-length wild-type MAAP, that inactivates the membrane-associated accessory protein (MAAP) mRNA translation-initiation codon and/or introduces at least one stop codon to stop translation of full-length wild-type MAAP as described herein. Preferably, stability is increased by at least 10%. In preferred embodiments, stability is increased by at least 15%, at least more preferably at least about 20%, more preferably at least about 25%, more preferably at least about 50%. In further preferred embodiments, stability is increased by at least about 60%, at least about 75%, more preferably at least about 80%, more preferably at least about 85%, more preferably at least about 90%, most preferably at least about 95%.

An AAV genome, or AAV vector, according to the invention can be replicated and packaged into viral particles, in particular infectious viral particles, when present in a suitable producer cell and in the presence of AAV Rep and Cap proteins. Also provided is therefore an adeno-associated virus comprising an AAV genome or AAV vector of the invention.

As used herein, “adeno-associated virus” or “AAV” refers to a viral particle composed of at least one AAV capsid protein VP1, VP2 and/or VP3, preferably all of the capsid proteins of a wild-type AAV, and an encapsidated polynucleotide AAV genome or AAV vector. An AAV of the invention is typically a recombinant AAV. In preferred embodiments the AAV is a non-naturally occurring AAV. The AAV may comprise one or more heterologous polynucleotides, i.e. a polynucleotide other than a wild-type AAV polynucleotide, such as a transgene. An example of a transgene is a therapeutic gene. A “therapeutic gene” as used herein refers to a gene that, when expressed in a cell, produced a gene product that confers a beneficial effect on the cell, tissue or animal in which it is expressed.

The AAV of the invention can be replication competent or replication incompetent. “Replication competent” means that the virus or viral particle is infectious and is capable of being replicated in a suitable infected cell. In preferred embodiments, the AAV of the invention is replication incompetent.

In some embodiments, the AAV genome comprises a polynucleotide that is operably linked to a promoter sequence, in particular a promoter sequence that drives expression of the polynucleotide in a host cell. As used herein “operably linked” when referring to polynucleotide that is operably linked to a promoter sequence, means the polynucleotide sequence is placed in a functional relationship with the promoter, i.e. the promoter is operably linked to a polynucleotide sequence if the promoter effects the transcription of the sequence.

In preferred embodiments, the adeno-associated virus according to the invention has increased infectivity. In particular, infectivity is increased as compared to infectivity of an AAV comprising an AAV genome that is identical to AAV genome of the invention with the exception that it lacks a mutation that reduces expression of full-length wild-type MAAP, that inactivates the membrane-associated accessory protein (MAAP) mRNA translation-initiation codon and/or introduces at least one stop codon to stop translation of full-length wild-type MAAP as described herein. Preferably, infectivity is increased by at least 10%. In preferred embodiments, infectivity is increased by at least 15%, at least more preferably at least about 20%, more preferably at least about 25%, more preferably at least about 50%. In further preferred embodiments, infectivity is increased by at least about 60%, at least about 75%, more preferably at least about 80%, more preferably at least about 85%, more preferably at least about 90%, most preferably at least about 95%.

In preferred embodiments, the adeno-associated virus according to the invention has increased stability. In particular, stability is increased as compared to stability of a and AAV comprising an AAV genome that is identical to AAV genome of the invention with the exception that it lacks a mutation that reduces expression of full-length wild-type MAAP, that inactivates the membrane-associated accessory protein (MAAP) mRNA translation-initiation codon and/or introduces at least one stop codon to stop translation of full-length wild-type MAAP as described herein. Preferably, stability is increased by at least 10%. In preferred embodiments, stability is increased by at least 15%, at least more preferably at least about 20%, more preferably at least about 25%, more preferably at least about 50%. In further preferred embodiments, stability is increased by at least about 60%, at least about 75%, more preferably at least about 80%, more preferably at least about 85%, more preferably at least about 90%, most preferably at least about 95%.

In a further aspect is provided a producer cell that produces adeno-associated virus, the producer cell comprising an adeno-associated virus genome of the invention.

In a further aspect is provided is a producer cell that produces adeno-associated virus, the producer cell substantially free of polypeptide having:
(a) at least 50% homology to any 30 contiguous residues of MAAP consensus polypeptide sequence SEQ. ID NO. 11;
(b) at least 95% homology to any 15 contiguous residues of MAAP consensus polypeptide sequence SEQ. ID NO. 11;
(c) at least 50% homology to any 10 contiguous residues of MAAP consensus polypeptide sequence SEQ. ID NO. 11 residue numbers 94 to 120. The producer cell is preferably free of polypeptides as detailed herein above.

As used herein a “producer cell” is also referred to as a “host cell”. The terms “producer cell” and “host cell” as used herein refers to any cell capable of being infected or transduced by an AAV, in particular an AAV of the invention. As used herein, “transduce” or “transduction” refers to the introduction of one or more polynucleotides into a cell by a virus or viral vector. As used herein, the term “suitable host cell” means a host cell that allows expression of proteins encoded by the AAV genome if infected by a viral particle, vector or plasmid comprising an AAV genome. In preferred embodiments, the producer cell is a genetically engineered producer cell.

Introduction or inclusion of an AAV genome or vector into a cell or producer to produce viral particles can be achieved by any conventional method in the art, which methods are well known to a skilled person. In preferred embodiments, the cell or producer cell is transduced with the AAV genome. For instance, an AAV expression vector comprising an AAV genome according to the invention can be introduced into a producer cell together with an AAV helper construct comprising a polynucleotide sequence encoding AAV capsid proteins and other AAV helper functions, including replication proteins and packaging proteins, necessary for infection and/or replication, followed by culturing of the producer cells to produce the AAV. Alternatively, an AAV genome of the invention comprises all nucleotides sequences and proteins necessary for infection and replication of viral particles. A suitable method is described in the examples herein.

The terms “producer cell” and “host cell” encompasses to any eukaryotic or prokaryotic cell (e.g., bacterial cells such as E. coli, yeast cells, mammalian cells, avian cells, amphibian cells, plant cells, fish cells, and insect cells). Host cells may be in vitro or in vivo, e.g. located in a transgenic animal. In preferred embodiments, the producer cell is eukaryotic. In preferred embodiments, the producer cell is mammalian. In preferred embodiments, the producer cell is a human cell. In preferred embodiments, the producer cell is selected from of yeast cells and insect cells. A skilled person is well capable of selecting a suitable producer cell for producing adeno-associated virus. One example of suitable producer cells are 293T cells. Other examples of producer cells include, but are not limited to, HeLa cell, COS cell, COS-1 cell, COS-7 cell, HEK293 cell, A549 cell, BHK cell, BSC-1 cell, BSC-40 cell, Vero cell, Sf9 cell, Sf-21 cell, Tn-368 cell, BTI-Tn-5B1-4 (High-Five) cell, Saos cell, C2C12 cell, L cell, HT1080 cell, HepG2 cell, WEHI cell, 3T3 cell, 10T1/2 cell, MDCK cell, BMT-10 cell, WI38 cell, and primary mammalian fibroblast, hepatocyte or myoblast cells.

In a further aspect is provided a producer cell comprising an adeno-associated virus genome, the producer cell able to express adeno-associated virus, the producer cell substantially free of full-length functional MAAP. In preferred embodiments, the adeno-associated virus genome has a mutation that interferes with the expression of full-length, wild-type functional MAAP. In preferred embodiments, the producer cell comprises interfering RNA that interferes with the expression of full-length, wild-type functional MAAP. In preferred embodiments, comprises a monoclonal antibody directed against MAAP that binds to MAAP and impairs the function of MAAP.

In preferred embodiments, the producer cell of the invention has increased viral production. In particular, viral production is increased as compared to viral production by a same producer cell in which an AAV genome is introduced that is identical to an AAV genome of the invention with the exception that it lacks a mutation that reduces expression of full-length wild-type MAAP, that inactivates the membrane-associated accessory protein (MAAP) mRNA translation-initiation codon and/or introduces at least one stop codon to stop translation of full-length wild-type MAAP as described herein. Preferably, viral production is increased by at least 10%. In preferred embodiments, viral production is increased by at least 15%, at least more preferably at least about 20%, more preferably at least about 25%, more preferably at least about 50%. In further preferred embodiments, viral production is increased by at least about 60%, at least about 75%, more preferably at least about 80%, more preferably at least about 85%, more preferably at least about 90%, most preferably at least about 95%.

In a further aspect is provided a method for producing adeno-associated virus, the method comprising: obtaining an adeno-associated virus genome, and then introducing said genome into a cell to create the producer cell of the invention, and then culturing said producer cell whereby said producer cell produces adeno-associated virus. In preferred embodiments, the method further comprises harvesting said adeno-associated virus. In preferred embodiments, the adeno-associated virus genome is an adeno-associated virus genome according to the invention.

In a further aspect is provided a method for producing adeno-associated virus, the method comprising culturing the producer cell of the invention, whereby the producer cell produces adeno-associated virus. In preferred embodiments, the method further comprises harvesting said adeno-associated virus.

The adeno-associated virus produced with a method of the invention may comprise one or more heterologous polynucleotides, i.e. a polynucleotide other than a wild-type AAV polynucleotide, such as a transgene. In preferred embodiments, the adeno-associated virus, in particular the harvested adeno-associated virus, comprises a transgene. An example of a transgene is a therapeutic gene.

In preferred embodiments, a method of the invention for producing AAV comprises culturing the producer cells for more than 24 hours, in particular culturing the producer cells for more than 24 hours before AAV is harvested. In preferred embodiments, the producer cells are cultured for at least 30 hours, in particular culturing the producer cells for at least 30 hours before AAV is harvested. In preferred embodiments, the producer cells are cultured for at least 36 hours, in particular culturing the producer cells for at least 36 hours before AAV is harvested. In preferred embodiments, the producer cells are cultured for at least 48 hours, in particular culturing the producer cells for at least 48 hours before AAV is harvested. In preferred embodiments, the producer cells are cultured for at least 60 hours, in particular culturing the producer cells for at least 60 hours before AAV is harvested. In preferred embodiments, the producer cells are cultured for at least 72 hours, in particular culturing the producer cells for at least 72 hours before AAV is harvested.

In preferred embodiments, the producer cell produces virus preparation wherein the ratio of the number of capsids containing the gene or genome of interest to the number of total physical capsids is at least as high as the ratio of the number of capsids containing the gene or genome of interest to the number of total physical capsids produced by a similar cell containing a wild-type adeno-associated virus genome. In preferred embodiments, the genome of interest is preferably an AAV genome according to the invention. In preferred embodiments, the gene of interest is an AAV gene, in particular a MAAP gene that has a mutation that inactivates the membrane-associated accessory protein (MAAP) mRNA translation-initiation codon or introduces at least one stop codon to stop translation of full-length wild-type MAAP according to the invention. In other preferred embodiments, the gene of interest is a heterologous gene, in particular a transgene. An example of a transgene is a therapeutic gene.

In preferred embodiments, the producer cell produces virus having a ratio of full: empty virus capsids least as high as does a similar cell infected with a wild-type adeno-associated virus genome. In preferred embodiments, the producer cell produces virus having a ratio of full: empty virus capsids 30% higher than does a similar cell infected with wild-type adeno-associated virus.

In preferred embodiments, the producer cell produces virus having at least as many viral genomes/mL as does a similar cell infected with wild-type adeno-associated virus. In preferred embodiments, the producer cell produces virus having at least four times as many viral genomes/mL as does a similar cell infected with wild-type adeno-associated virus.

Further provided is a method of increasing stability, increasing capsid integrity, or reducing capsid degradation of an adeno-associated virus (AAV), comprising including in the AAV the adeno-associated virus genome of the invention.

Further provided is a method of increasing the proportion of AAV capsids containing a gene or genome of interest, comprising including in the AAV the adeno-associated virus genome of the invention and the gene or genome of interest.

Further provided is a method of the increasing the viral titre (viral genomes/mL) of a producer cell producing an AAV, comprising including in the AAV the adeno-associated virus genome of the invention and introducing the AAV in the producer cell.

In preferred embodiments, the producer cell is cultured for at least 30 hours.

In preferred embodiments, the producer cell is cultured for at least 36 hours, 48 hours, 72 hours or 96 hours.

Further provided is a method for increasing the retention of viral genomes or viral particles in a producer cell producing an AAV, comprising including in the AAV the adeno-associated virus genome of the invention and introducing the AAV in the producer cell. In preferred embodiments, the method further comprises harvesting and/or purifying the viral genomes or viral particles from the producer cells, preferably substantially free of media.

Further provided is an adeno-associated virus produced by a method of the invention for producing adeno-associated virus. In preferred embodiments, the adeno-associated virus is an adeno-associated virus comprising an AAV genome according to the invention. An AAV of the invention is typically a recombinant AAV. In preferred embodiments the AAV is a non-naturally occurring AAV. The AAV may comprise one or more heterologous polynucleotides, i.e. a polynucleotide other than a wild-type AAV polynucleotide, such as a transgene.

Features may be described herein as part of the same or separate aspects or embodiments of the present invention for the purpose of clarity and a concise description. It will be appreciated by the skilled person that the scope of the invention may include embodiments having combinations of all or some of the features described herein as part of the same or separate embodiments.

### BRIEF DESCRIPTION OF THE FIGURES

The patent or application file contains at least one drawing executed in color. Copies of this patent or patent application publication with color drawing(s) will be provided by the Office upon request and payment of any necessary fee.

FIG. 1 shows the fluorescence intensity of cells transfected with various plasmids and as control with PEI alone.

FIG. 2 follows the expression over time of the Rep78/52, VPs, AAP and MAAP proteins during WT AAV production in 293T cells.

FIG. 3 shows AAV viral titers (expressed as viral genomes per mL when measured using ddPCR) 24 hours after infection of producer cells. Column 1: wild-type (wt) AAV serotype 2. Column 2 is MAAP with the first theoretical non-canonical start codon (CTG, coding for residue L1 on the full-length serotype 2 MAAP polypeptide, SEQ ID NO. 2) mutated to CGG. Column 3 is MAAP with residue Q9 on SEQ ID NO. 2 mutated to a stop codon. Column 4 is MAAP with residue S39 on SEQ ID NO. 2 mutated to a stop codon. Column 5 is MAAP with residues S33, S39 and S47 on SEQ ID NO. 2 each mutated to a stop codon. Columns 6-12 are MAAP with each of residues S65, E90, L100, W103, W105, L106 and L110 (respectively) on SEQ ID NO.2 mutated to a stop codon.

FIG. 4 shows AAV viral titers (expressed as viral genomes per mL when measured using ddPCR) 72 hours after infection of producer cells. Columns are as with preceding FIG. 3.

FIG. 5 shows MAAP over-expression effect on AAV viral genomes per mL at 24 h, Column 1 wild type MAAP gene; Column 2: MAAP with residues S33, S39 and S47 on the full-length MAAP polypeptide each mutated to a stop codon. Column 3: As column 1, but cells treated also with MAAP overexpressing plasmid; Column 4: As column 2, but cells treated also with MAAP overexpressing plasmid; Column 5: wt-AAV2 and GFP overexpressing plasmid treated cells; Column 6: as column 2, but with GFP plasmid.

FIG. 6 shows MAAP over-expression effect on AAV viral genomes per mL at 72 h post-transfection, Column 1 wild type MAAP gene; Column 2: MAAP with residues S33, S39 and S47 on the full-length MAAP polypeptide each mutated to a stop codon. Column 3: As column 1, but cells treated also with MAAP overexpressing plasmid; Column 4: As column 2, but cells treated also with MAAP overexpressing plasmid; Column 5: wt-AAV2 and GFP overexpressing plasmid treated cells; Column 6: as column 2, but with GFP plasmid.

FIG. 7 measures the amount of contaminant kanamycin-resistance gene (kan) DNA (from the plasmids used to make virus) relative to AAV viral genome packaged in viral capsids when measured 24 hours after infection of producer cells. Columns are as with preceding FIG. 3.

FIG. 8 measures the amount of contaminant kan DNA relative to AAV viral genome packaged in viral capsids when measured 72 hours after infection of producer cells. Columns are as with preceding FIG. 3.

FIG. 9 measures the amount of packaged contaminant kan DNA after or in the presence of MAAP over-expression relative to AAV viral genome packaged in viral capsids when measured 24 hours after infection of producer cells. Columns are as with preceding FIG. 5.

FIG. 10 measures the amount of packaged contaminant kan DNA after or in the presence of MAAP over-expression relative to AAV viral genome packaged in viral capsids when measured 72 hours after infection of producer cells. Columns are as with preceding FIG. 6.

FIG. 11 measures the amount of contaminant adenovirus serotype 5 E4 gene DNA (from the helper adenovirus plasmid that was used to make adeno-associated virus) relative to AAV viral genome packaged in AAV viral capsids when measured 24 hours after infection of producer cells. Columns are as with preceding FIG. 3.

FIG. 12 measures the amount of contaminant adenovirus serotype 5 E4 gene DNA (from the helper adenovirus that was used to make adeno-associated virus) relative to AAV viral genome packaged in AAV viral capsids when measured 72 hours after infection of producer cells. Columns are as with preceding FIG. 3.

FIG. 13 is a photograph of a Western blot measuring polypeptide expression at 24h in cells transfected with plasmid coding for wt-AAV (v1 and v7 denote different versions of an adenovirus-genome helper plasmid; v7 is smaller than v1), and for MAAP with stop codons newly-introduced at amino acid residue Nos. E90, L100, W103, W105, L106 or L110 (as shown on the full-length MAAP sequence of SEQ ID NO. 2), and a negative control. Top panel: Expression of alpha-tubulin. Middle panel: Expression of MAAP. Bottom panel: expression of full-length VP-1, -2 and -3 (upper bands) and their degradation products (lower bands).

FIG. 14 is a photograph of a Western blot measuring expression of VP-1, -2 and -3 (top panel), MAAP (middle panel) and alpha-tubulin (bottom panel). Column 1: molecular weight marker. Column 2: wild-type (wt) AAV2. Column 3 is MAAP with the first theoretical non-canonical start codon (CTG, coding for residue L1 on the full-length MAAP polypeptide, SEQ ID NO. 2) mutated to CGG. Column 4 is MAAP with residue Q9 on the full-length MAAP polypeptide mutated to a stop codon. Column 5 is MAAP with residue S39 on the full-length MAAP polypeptide mutated to a stop codon. Column 6 is MAAP with residues S33, S39 and S47 on the full-length MAAP polypeptide each mutated to a stop codon. Column 7 is negative control.

FIG. 15 compares the percentage of capsids that are empty (lacking the desired DNA payload) or full (having the desired DNA payload). Columns are as with FIG. 3 above.

FIG. 16 shows the impact of MAAP variants on rAAV vg titers. rAAV of serotypes 1, 2, 5, 6, 8 and 9 encoding mSeAP were produced using 2-plasmid system from Plasmid Factory or 3-plasmid systems. The viral genome titers were quantified. Within the 3-plasmid systems, cap genes encoding same capsid serotype but either wt-MAAP, MAAP-triple stop, and MAAP-S/L-100 were used for rAAV production. (A) rAAV yields expressed as vg·mL⁻¹ alongside mean and SD. Statistical significance between rAAVs produced with wt-MAAP and mutated MAAP were calculated using two-tailed, unpaired Student's T-tests.

FIG. 17 shows the effect of MAAP variants on rAAV genome packaging. rAAV of serotypes 2, 5, 6 and 8 encoding mSeAP were produced using a 2-plasmid system containing a cap gene coding for wt-MAAP, and 3-plasmid systems with the cap gene encoding wt-MAAP or MAAP variants. rAAV vg titers were quantified. In parallel, we quantified the total number of AAV capsids from the same samples by ELISA. We present the ratio of capsid containing rAAV genome versus total capsids, expressed as percentage. Percentages of rAAV capsids encoding the mSeAP transgene are shown accompanied by mean and SD. Statistical significance between rAAV produced with cap gene encoding wt-MAAP or mutated MAAP was calculated using two-tailed, unpaired Student's T-tests.

FIG. 18 shows MAAP variants modify the secretion profiles of rAAV. rAAV of serotypes 1, 2, 5, 6, 8 and 9 encoding mSeAP were produced using 2-plasmid system from Plasmid Factory or 3-plasmid systems and viral genome titers were quantified from the cell culture or from cell culture media. Within the 3-plasmid systems, cap genes encoding wt-MAAP; MAAP-triple stop, MAAP-S/L-100 were used for rAAV production. The mean percentages of vg titers in cell media in respect to vg titers in cell lysate are displayed as ‘secreted viral particles’ with SD. Statistical significance between rAAV of same capsid serotype but produced with wt-MAAP or MAAP variants was calculated using two-tailed, unpaired Student's T-tests.

FIG. 19 shows the MAAP phylogenetic tree. Phylogenetic tree of the MAAP protein sequence of primate AAVs. Nodes with bootstrap values above 75 are indicated with 4 different size of circles. The nomenclature is either the serotype name or a reference to the species in which the AAV was identified (hu, human; rh, rhesus macaque; pi, pigtailed macaque) followed by a serotype number.

FIG. 20 shows the sequences of SEQ ID NO's 1 to 29.

FIG. 21 A-J provides the nucleic acid sequences of cap genes encoding inter alia MAAP for AAV serotypes 1-10, respectively.

### EXAMPLES

### Example 1

Materials & Methods

Full-Length and Truncated MAAP

Doing an analysis of AAV, we identified a possible novel viral protein and several non-canonical start codons for its translation. We then discovered that one of these three non-canonical start codons in fact operates in wild-type AAV to initiate translation of a novel wild-type protein. SEQ ID NO's 1-10 provide the primary amino acid sequence for the wild type protein for AAV serotypes 1-10 respectively. The amino acid sequence for each of these serotypes is highly conserved at the C-terminal end. At the N-terminal end, AAV serotype 4 (SEQ ID NO. 4) and serotype 5 (SEQ ID NO. 5) wild-type proteins have a leading 15-25 amino acid residue sequence not seen in the other serotypes. SEQ ID NO. 11 provides the primary amino acid sequence for the theoretical consensus of all ten of these serotypes. We refer to these proteins collectively, and each one individually, as “MAAP”.¹ ¹ The same newly-discovered protein has recently and independently been described by Ogden P. J. et al., Comprehensive AAV Capsid Fitness Landscape Reveals A Viral Gene And Enables Machine-Guided Design, 366 Science 1139 (2019). Ogden et al. refers to this newly-discovered protein as “membrane-associated accessory protein” or “MAAP.”

The wild-type DNA sequence includes two further non-canonical start codons. One of these is AGG, (coding for amino acid residue 13 on the full-length polypeptide sequence of SEQ ID NO. 2). The other is ACG (coding for amino acid residue 14 on the full-length polypeptide sequence of SEQ ID NO. 2.

Virus Preparations

AAV Virus production was carried out as follows. 293T cells (European Collection of Cell Cultures 293T Number: 12022001) were grown in Dulbecco's modified Eagle medium (DMEM, Gibco 11965084) supplemented with 10% fetal bovine serum (FBS, Thermo Fisher 10091-148), supplemented with 2 mM 1-glutamine (Gibco, 25030-024), and penicillin-streptomycin (Gibco 15070-063).

Polyethyleneimine (PEI) transfections of AAV plasmid and adenovirus helper plasmid were performed on 293T cells in T25 flasks (60,000 cells/cm²). The PEI Pro™ (Polyplus Transfection, ref #115-100)/DNA weight ratio was maintained at 1:1 in serum-free DMEM medium. For AAV2, we used AAV2 plasmid and adenovirus helper plasmid in a 1:1 ratio at a total of 350 ng/cm². Other AAV serotypes may be similarly used with the appropriate ratio of AAV plasmid to helper plasmid.

To analyze the relative usage of the non-canonical start codons, we point-mutated the first non-canonical start codon from CTG to CGG, inactivating the codon as a potential start codon.

To evaluate the function of this novel protein, we created artificially-truncated mutant forms of it. Designing these mutations was non-trivial because the same DNA sequence is used to express VP1, a viral protein that is critical for virus entry into a host cell and intracellular delivery of the viral genome to the infected cell nucleus. Thus, we needed to make point mutations that create codons that stop MAAP translation, yet do not perturb transcription of, nor the amino acid sequence of, VP1. We identified eleven suitable mutation sites. These new stop codons theoretically truncate MAAP (here, serotype 2, SEQ ID. NO. 2) translation at amino acid residue Nos. Q9, S33, S39, S47, S65, E90, L100, W103, W105, L106 and L110. The Table below provides a more complete list the different plasmids used in the study, the point-mutations we performed on the MAAP gene, and their impacts on the resulting MAAP and VP1 amino acid sequences.

TABLE 1
Plasmids used
Plasmids Used

| | | MAAP | MAAP | | VP1 | |
|---|---|---|---|---|---|---|
| # | Name | wt | mutated | VP1 wt | mutated | Use |
| p0059 | p0059- | | | | | Stuffer plasmid |
| | pDEST- | | | | | |
| | eGFP | | | | | |
| p0088 | p0088- | — | — | — | — | Helper plasmid |
| | pHelper- | | | | | used for WT AAV2 |
| | Ad5-Lio | | | | | and rAAV2 |
| | | | | | | production. |
| | | | | | | Encodes VAI, |
| | | | | | | VAII, E2, E4 |
| | | | | | | regions of |
| | | | | | | Adenovirus 5. |
| p0108 | p0108- | WT | — | WT | — | Reference for WT |
| | pGRG25- | sequence | | sequence | | AAV2 production |
| | AAV2WT | | | | | (Savy et al., 2018) |
| p0188 | p0188- | Gln 9 | stop 9 | Ala 35 | Val 35 | Study of MAAP |
| | AAV2WT- | (CAG) | (TAG) | (GCA) | (GTA) | start codon |
| | MAAP | | | | | |
| | Q16 -> | | | | | |
| | stop | | | | | |
| p0189 | p0189- | Ser 33 | Stop 33 | Leu 59 | Leu 59 | Study of MAAP |
| | AAV2WT- | (TCG)- | (TAG)- | (CTC)- | (CTA)- | inactivation |
| | MAAP | Ser 39 | Stop 39 | Val 65 | Val 65 | |
| | S40-S46- | (TCA)- | (TGA)- | (GTC)- | (GTA)- | |
| | S54 -> | Ser 47 | Stop 47 | Leu 72 | Leu 72 | |
| | stops | (TCG) | (TAG) | (CTC) | (CTA) | |
| p0190 | p0190- | Ser 33 | Stop 33 | Leu 59 | Leu 59 | Study of MAAP |
| | AAV2WT- | (TCG) | (TAG) | (CTC) | (CTA) | inactivation |
| | MAAP | | | | | |
| | S40 -> | | | | | |
| | stop | | | | | |
| p0191 | p0191- | Ser 39 | Stop 39 | Val 65 | Val 65 | Study of MAAP |
| | AAV2WT- | (TCA) | (TGA) | (GTC) | (GTA) | inactivation |
| | MAAP | | | | | |
| | S46 -> | | | | | |
| | stop | | | | | |
| p0192 | p0192- | Ser 47 | Stop 47 | Leu 72 | Leu 72 | Study of MAAP |
| | AAV2WT- | (TCG) | (TAG) | (CTC) | (CTA) | inactivation |
| | MAAP | | | | | |
| | S54 -> | | | | | |
| | stop | | | | | |
| p0193 | p0193- | Ser 65 | Stop 65 | Leu 91 | Leu 91 | Study of MAAP |
| | AAV2WT- | (TCA) | (TGA) | (CTC) | (CTC) | inactivation |
| | MAAP | | | | | |
| | S72 -> | | | | | |
| | stop | | | | | |
| p0194 | p0194- | Glu 90 | Stop 90 | Arg 116 | Leu 116 | Study of MAAP |
| | AAV2WT- | (GAG) | (TAG) | (CGA) | (CTA) † | nuclear |
| | MAAP | | | | | localization signal |
| | E97 -> | | | | | |
| | stop | | | | | |
| p0195 | p0195- | Leu 100 | Stop 100 | Leu 126 | Leu 126 | Study of MAAP |
| | AAV2WT- | (TTG) | (TAG) | (CTT) | (CTA) | nuclear |
| | MAAP | | | | | localization signal |
| | L107 -> | | | | | |
| | stop | | | | | |
| p0196 | p0196- | Trp 103 | Stop 103 | Leu 129 | Leu 129 | Study of MAAP |
| | AAV2WT- | (TGG) | (TAG) | (CTG) | (CTA) | nuclear |
| | MAAP | | | | | localization signal |
| | W110 -> | | | | | |
| | stop | | | | | |
| p0197 | p0197- | Trp 105 | Stop 105 | Leu 131 | Leu 131 | Study of MAAP |
| | AAV2WT- | (TGG) | (TAG) | (CTG) | (CTA) | nuclear |
| | MAAP | | | | | localization signal |
| | W112 -> | | | | | |
| | stop | | | | | |
| p0198 | p0198- | Leu 106 | Stop 106 | Val 132 | Val 132 | Study of MAAP |
| | AAV2WT- | (TTG) | (TAG) | (GTT) | (GTA) | nuclear |
| | MAAP | | | | | localization signal |
| | L113 -> | | | | | |
| | stop | | | | | |
| p0199 | p0199- | Leu 110 | Stop 110 | Val 136 | Val 136 | Study of MAAP |
| | AAV2WT- | (TTA) | (TGA) | (GTT) | (GTG) | nuclear |
| | MAAP | | | | | localization signal |
| | L117 -> | | | | | |
| | stop | | | | | |
| p0200 | p0200- | WT | WT | insertion | | Study of MAAP |
| | AAV2WT- | sequence | sequence | of GFP | | start codon- |
| | MAAP- | | | fused to | | MAAP-GFP fusion |
| | GFP | | | MAAP | | in WT-AAV |
| | | | | takes | | plasmid context |
| | | | | place | | |
| | | | | after | | |
| | | | | VP1- | | |
| | | | | P145 | | |
| p0201 | p0201- | Gln 9 | Stop 9 | Ala 35 | Val 35 | Study of MAAP |
| | AAV2WT- | (CAG) | (TAG) | (GCA) | (GTA) | start codon- |
| | MAAP- | | | | | MAAP-GFP fusion |
| | GFP Q16 | | | | | in WT-AAV |
| | -> stop | | | | | plasmid context |
| p0202 | p0202- | Ser 33 | Stop 33 | Leu 59 | Leu 59 | Study of MAAP |
| | AAV2WT- | (TCG) | (TAG) | (CTC) | (CTA) | inactivation- |
| | MAAP- | | | | | MAAP-GFP |
| | GFP S40 | | | | | fusion |
| | -> stop | | | | | |
| p0203 | p0203- | Ser 39 | Stop 39 | Val 65 | Val 65 | Study of MAAP |
| | AAV2WT- | (TCA) | (TGA) | (GTC) | (GTA) | inactivation- |
| | MAAP- | | | | | MAAP-GFP |
| | GFP S46 | | | | | fusion |
| | -> stop | | | | | |
| p0204 | p0204- | Ser 47 | Stop 47 | Leu 72 | Leu 72 | Study of MAAP |
| | AAV2WT- | (TCG) | (TAG) | (CTC) | (CTA) | inactivation- |
| | MAAP- | | | | | MAAP-GFP |
| | GFP S54 | | | | | fusion |
| | -> stop | | | | | |
| p0205 | p0205- | Ser 33 | Stop 33 | Leu 59 | Leu 59 | Study of MAAP |
| | AAV2WT- | (TCG)- | (TAG)- | (CTC)- | (CTA)-Val | inactivation- |
| | MAAP- | Ser 39 | Stop 39 | Val 65 | 65 (GTA)- | MAAP-GFP |
| | GFP S40- | (TCA)- | (TGA)- | (GTC)- | Leu 72 | fusion |
| | 46-54 -> | Ser 47 | Stop 47 | Leu 72 | (CTA) | |
| | stops | (TCG) | (TAG) | (CTC) | | |
| p0206 | p0206- | Leu 1 | Arg 1 | Pro 27 | Pro 27 | Study of MAAP |
| | AAV2WT- | (CTG) | (CGG) | (CCT) | (CCG) | start codon- |
| | MAAP- | | | | | MAAP-GFP fusion |
| | GFP | | | | | in WT-AAV |
| | start1 | | | | | plasmid context |
| | Leu8 -> | | | | | |
| | Arg | | | | | |
| p0223 | p0223- | Leu 1 | Arg 1 | Pro 27 | Pro 27 | Study of MAAP |
| | AAV2WT- | (CTG) | (CGG) | (CCT) | (CCG) | start codon |
| | MAAP- | | | | | |
| | start 1 | | | | | |
| | Leu8 -> | | | | | |
| | Arg | | | | | |
| p0226 | p0226- | WT | — | WT | — | Study of MAAP |
| | AAV2WT- | sequence | | sequence | | interaction with |
| | AAP- | | | | | AAP |
| | S13-W46 | | | | | |
| | -> stops | | | | | |
| p0230 | p0230- | WT | — | WT | — | Reference for WT |
| | pUC-K- | sequence | | sequence | | AAV2 production |
| | AAV2WT- | | | | | |
| | Reverse | | | | | |
| p0273 | p0273- | | | | | Helper plasmid |
| | Ad5-Lio- | | | | | used for WT AAV2 |
| | v7 | | | | | and rAAV2 |
| | | | | | | production. |
| | | | | | | Encodes VAI, |
| | | | | | | VAII, E2, E4 |
| | | | | | | regions of |
| | | | | | | Adenovirus 5. |
| p0280 | p0280- | | | | | Expression of |
| | MAAP | | | | | MAAP driven by |
| | | | | | | ATG codon, under |
| | | | | | | CMV enhancer- |
| | | | | | | CMV promoter- |
| | | | | | | SV40 intron |
| | | | | | | promoter sequence |
| p0283 | p0283- | | | | | Expression of |
| | MAAP- | | | | | MAAP-GFP driven |
| | GFP | | | | | by ATG codon, |
| | | | | | | under CMV |
| | | | | | | enhancer-CMV |
| | | | | | | promoter-SV40 |
| | | | | | | intron promoter |
| | | | | | | sequence |
| p0326 | p0326- | | | | | Expression of |
| | MAAP | | | | | MAAP initiated at |
| | start 2 | | | | | Start 2 (R13) |
| | | | | | | modified to ATG |
| | | | | | | codon, under CMV |
| | | | | | | enhancer-CMV |
| | | | | | | promoter-SV40 |
| | | | | | | intron promoter |
| | | | | | | sequence |
| p0331 | p0331- | | | | | |
| | AAP | | | | | |

# = Plasmid code number.
wt = wild-type.
† = This mutation modifies the VP1 amino acid sequence at the same time as it introduces a stop codon in MAAP.

TABLE 2
Plasmids used for AAV production
Plasmids Used for AAV Production

| | | | MAAP | | |
|---|---|---|---|---|---|
| # | Name | MAAP wt | mutated | n | t |
| | | | | 3 | 24 h; 72 h |
| | p0223-AAV2WT-MAAP- start1 Leu8 −> Arg | | | | |
| p0188 | p0188-AAV2WT-MAAP Q16 −> stop | Gln 9 | stop 9 | 3 | 24 h; 72 h |
| | | (CAG) | (TAG) | | |
| p0189 | p0189-AAV2WT-MAAP S40-S46-S54 −> stops | Ser 33 | Stop 33 | 3 | 24 h; 72 h |
| | | (TCG) - | (TAG) - | | |
| | | Ser 39 | Stop 39 | | |
| | | (TCA) - | (TGA) - | | |
| | | Ser 47 | Stop 47 | | |
| | | (TCG) | (TAG) | | |
| p0190 | p0190-AAV2WT-MAAP S40 −> stop | Ser 33 | Stop 33 | — | — |
| | | (TCG) | (TAG) | | |
| p0191 | p0191-AAV2WT-MAAP S46 −> stop | Ser 39 | Stop 39 | 3 | 24 h; 72 h |
| | | (TCA) | (TGA) | | |
| p0192 | p0192-AAV2WT-MAAP S54 −> stop | Ser 47 | Stop 47 | — | — |
| | | (TCG) | (TAG) | | |
| no sample; | p0193-AAV2WT-MAAP S72 −> stop | | | 3 | 72 h |
| no WB | | | | | |
| p0194 | p0194 - AAV2WT-MAAP E97 −> stop | Glu 90 | Stop 90 | 3 | 24 h; 72h |
| | | (GAG) | (TAG) | | |
| p0195 | p0195 - AAV2WT-MAAP L107 −> stop | Leu 100 | Stop 100 | 3 | 24 h; 72 h |
| | | (TTG) | (TAG) | | |
| p0196 | p0196 - AAV2WT-MAAP W110 −> stop | Trp 103 | Stop 103 | 2 | 24 h |
| | | (TGG) | (TAG) | | |
| p0197 | p0197 - AAV2WT-MAAP W112 −> stop | Trp 105 | Stop 105 | 2 | 24 h |
| | | (TGG) | (TAG) | | |
| p0198 | p0198 - AAV2WT-MAAP L113 −> stop | Leu 106 | Stop 106 | 3 | 24 h; 72 h |
| | | (TTG) | (TAG) | | |
| p0199 | p0199 - AAV2WT-MAAP L117 −> stop | Leu 110 | Stop 110 | 3 | 24 h; 72 h |
| | | (TTA) | (TGA) | | |
| | WB 24 h samples | | | | |
| | p0230-pUC-K-AAV2WT-Reverse | | | | |
| | p0189-AAV2WT-MAAP S40-S46-S54 −> stops | | | | |
| | p0194 - AAV2WT-MAAP E97 −> stop | | | | |
| | p0195 - AAV2WT-MAAP L107 −> stop | | | | |
| | p0196 - AAV2WT-MAAP W110 −> stop | | | | |
| | p0197 - AAV2WT-MAAP W112 −> stop | | | | |
| | p0198 - AAV2WT-MAAP L113 −> stop | | | | |
| | p0199 - AAV2WT-MAAP L117 −> stop | | | | |
| | neg | | | | |
| | p0230-pUC-K-AAV2WT-Reverse | | | 3 | 24 h; 72 h |
| | p0223-AAV2WT-MAAP- start1 Leu8 −> Arg | | | | |
| | p0188-AAV2WT-MAAP Q16 −> stop | | | | |
| | p0191-AAV2WT-MAAP S46 −> stop | | | | |
| | p0189-AAV2WT-MAAP S40-S46-S54 −> stops | | | | |

# = Plasmid code number.
n = number of experimental runs.
t = time (hours) after producer cell infection for virus harvest

For fluorescence-activated cell sorting, we used the following plasmids:

TABLE 3
Plasmids Used in Fluorescence-Activated Cell Sorting
Plasmids Used in Fluorescence-Activated Cell Sorting

| # | name | MAAP wt | MAAP mutated |
|---|---|---|---|
| p0200 | p0200-AAV2WT-MAAP-GFP | WT sequence | WT sequence |
| p0201 | p0201-AAV2WT-MAAP-GFP Q16 −> stop | Gln 9 (CAG) | Stop 9 (TAG) |
| p0202 | p0202-AAV2WT-MAAP-GFP S40 −> stop | Ser 33 (TCG) | Stop 33 (TAG) |
| p0203 | p0203-AAV2WT-MAAP-GFP S46 −> stop | Ser 39 (TCA) | Stop 39 (TGA) |
| p0204 | p0204-AAV2WT-MAAP-GFP S54 −> stop | Ser 47 (TCG) | Stop 47 (TAG) |
| p0205 | p0205-AAV2WT-MAAP-GFP S40-46-54 −> stops | Ser 33 (TCG) - Ser 39 | Stop 33 (TAG) - Stop |
| | | (TCA) -Ser 47 (TCG) | 39 (TGA) - Stop 47 (TAG) |
| p0206 | p0206-AAV2WT-MAAP-GFP | Leu 1 (CTG) | Arg 1 (CGG) |
| | start1 Leu8 −> Arg | | |

# = Plasmid number
MAAP original = MAAP original codon and amino acid
MAAP final = MAAP final mutation

Virus was harvested 24 h and 72 h after transfection.

For viral genome titer determination and AAV capsid ELISA samples, virus was harvested using Triton-X-100 buffer (0.5% Triton-X-100 (Sigma-Aldrich, refmX100-1L) and 2 mM MgCl2 (Merck, ref #E13980)) in 1×phosphate-buffered saline (PBS, Gibco, ref #18912-014) and Denarase (50 U/ml, c-Lecta, ref #20804-5M). Lysis buffer was added to the media and cells were incubated for 2 h at 37° C. before cell lysate was collected.

For samples processed for Western blot, virus was harvested as follows. Cells were detached using Tryple Select™ (Gibco, ref #12563-011) and suspended in 1×PBS (Gibco, rem #14190-094). Cells were pelleted by centrifugation (500 g, 5 min). Cell pellet was washed with 1×PBS and centrifugation was repeated. Cells were re-suspended in radio-immunoprecipitation assay (RIPA, Thermo Scientific, refm89901) buffer containing Proteinase Inhibitor Cocktail (cOmplete™, Roche, ref #1169749800). Samples were incubated on ice for 20 min and centrifuged at 20,000 g for 15 min. Supernatant was collected.

TABLE 4
Primers And Probes Used In The Study

| ID | Sequence |
|---|---|
| Rep2-PRB | /56-FAM/CCCGTGTCA/ZEN/GAATCTCAACCCGTT/ |
| | 3IABKFQ/ |
| Rep2-FWD | CTTCACTCACGGACAGAAAGA |
| Rep2-REV | CTGGCACCTTTCCCATGATA |
| Ad5-E4-PRB | /56-FAM/ACCCAGCCA/ZEN/ACCTACACATTCGTT/3IABKFQ/ |
| Ad5-E4-FWD | CATCCACCACCGCAGAATAA |
| Ad5-E4-REV | ACATGGTTCTTCCAGCTCTTC |
| Kan-PRB | /56-FAM/TCGCACCTG/ZEN/ATTGCCCGACATTAT/3IABKFQ/ |
| Kan-FWD | ATCGGGCTTCCCATACAATC |
| Kan-REV | GCTCTAGGCCGCGATTAAA |

Notes:
56-FAM, ZEN and 3IABKFQ are imaging agents.

Quantification of AAV and Contaminating Sequences by Droplet Digital PCR

To obtain droplet digital PCR (ddPCR) AAV viral genome (vg) titers, crude preparations of virus were first treated with DNaseI (0.01 U/μl, Invitrogen, ref #18047-019) and then Proteinase K (0.1 μg/μl, Roche, ref #03115879001), and viral titers were obtained by ddPCR amplification (QX200, Bio-Rad) with appropriate primers. For example, for AAV2, we used primers for Rep2-FWD and Rep2-REV, and probe Rep2-PRB to detect the AAV replicase region.

To assess levels of unwanted, contaminating DNA originating from the AAV plasmid backbone and Adenovirus helper plasmid backbone and packaged into AAV capsid, ddPCR was performed using appropriate primers. For example, to detect contamination by the kanamycin-resistance gene present on the plasmid backbone, we used primers for Kan-FWD and Kan-REV, and probe Kan-PRB for the kanamycin resistance gene. The adenovirus E4 (Ad5-E4) region set of primers (Ad5-E4-FWD; Ad5-E4-REV) and probe (Ad5-E4-PRB) was used to quantify the Adenovirus helper plasmid. All primers and probes were ordered from Integrated DNA Technologies.

For mastermix generation, primers (900 nM) and probe (250 nM) were diluted in 2×ddPCR supermix for Probes (no dUTP, Bio-Rad, ref #1863025) and nuclease free water (Thermo Scientific, ref #R0582). The Table above provides a list of primers and probes used in the study. Other primers and probes may be similarly used for different AAV serotypes or to probe for different contaminant DNA.

ELISA

To determine the ratios of capsids containing AAV genomes versus total AAV capsids, A20 capsid ELISAs were performed on serial dilutions of the virus preparation with the AAV titration ELISA kit (Progen, ref #PRATV) according to the manufacturer's instructions.

MAAP and AAP Antisera

Polyclonal anti-MAAP antiserum was obtained from the immunization of rabbit with the peptide KKIRLLGATSDEQSSRRKRG (SEQ ID NO 28), conjugated to a carrier before immunization (Davids Biotechnologie GmbH, Germany).

Polyclonal anti-AAP antiserum was obtained from the immunization of Guinea pig with peptide RSTSSRTSSARRIKDASRR (SEQ ID NO 29), conjugated to a carrier before immunization. Antisera were affinity purified (Davids Biotechnologie GmbH, Germany).

Western Blotting

Sample was denatured using 2-mercaptoethanol (10%, Sigma-Aldrich) in Laemmi sample buffer (Bio-Rad, re 1610747). A constant volume of each sample was run on Mini-Protean TGX gels (4-10%, Bio-Rad). Proteins were transferred to 0.2 μm PVDF membrane (Trans-Blot Turbo Transfer Pack, Bio-Rad) and stained with selected primary antibody (table) overnight. Proteins were detected with horseradish peroxydase (HRP) conjugated secondary antibody, and visualized using ChemiDoc (Bio-Rad).

TABLE 5
Western blot analysis: antibodies and dilutions used
Western blot analysis: antibodies and dilutions used

| Detected | | | | |
|---|---|---|---|---|
| protein | Primary antibody | 1° dil | Secondary antibody | 2° dil |
| AAV | 303.9 (Progen) | 1:250 | Goat anti-mouse IgG | 1:3000 |
| replicase | | | (H + L)-HRP conjugate (Bio- | |
| | | | Rad) | |
| AAV | B1 (Progen) | 1:250 | Goat anti-mouse IgG | 1:3000 |
| capsid | | | (H + L)-HRP conjugate (Bio- | |
| proteins | | | Rad) | |
| MAAP | GAL-KKI | 1 μg/mL | Goat anti-rabbit IgG | 1:3000 |
| | (Davids Biotechnologie) | | (H + L)-HRP conjugate (Bio- | |
| | | | Rad) | |
| AAP | GAL-RST | 3 μg/mL | Anti-guinea pig IgG (H + L)- | 1:1000 |
| | (Davids Biotechnologie) | | HRP conjugate (Sigma) | |
| α-tubulin | α-tubulin HRP | 1:1000 | NA | NA |
| | conjugated mouse | | | |
| | monoclonal IgG | | | |

1° dil = primary dilution;
2° dil = secondary dilution
α-tubulin HRP conjugated mouse monoclonal IgG from Santa Cruz Biotechnology.

### Statistical Analysis

Statistical comparison were performed using one-way analysis of variance (ANOVA), followed by a comparison of the wt-AAV2 reference against the other AAV2 assayed, performed using Dunnett's Multiple Comparison Test. The statistical tests were performed using GraphPad™ software (Prism).

### Results

### MAAP Translation Initiates at A CTG Codon

After detecting a possible novel viral protein, we analyzed the genome of wt AAV to identify potential non-ATG (non-canonical) initiation codons. Our review revealed at least three different non-canonical triplets that could theoretically initiate translation. Each of these three differs by only one base compared to the canonical ATG start codon. Thus, each can theoretically initiate translation.

We found that the first CTG encountered on the MAAP reading frame is the principal translation initiation codon for MAAP. The CTG translates to a leucine in MAAP (L1 on the full-length protein). On VP1 frame (−1 to MAAP), at this site is CCT that translates to P27.

To analyze non-canonical start codon usage in the context of the wild-type genome, we mutated the first potential non-canonical start codon of MAAP (CTG, L1 on the full-length protein) to CGG (translating to R1). This abolished its potential start codon function. In response, we found that MAAP production fell to levels undetectable using Western blot.

We thus confirmed our results several different ways. First, we introduced a stop codon in place of MAAP-Q9, between the first (CTG) and the second (AGG) potential non-canonical start codons. In response, we found that MAAP protein production fell to levels undetectable using Western blot.

Similarly, we introduced a stop codon in place of MAAP-S39. In response, we found that MAAP protein production fell to levels undetectable using Western blot.

Similarly, we placed three consecutive stop codons at MAAP amino acid residues S33, S39 and S47. In response, we found that MAAP production fell to levels undetectable using Western blot.

Our results differ from what Ogden (2019) observed. In their study, Ogden (2019) observed protein expression (perhaps in truncated form) when the first CTG start codon was mutated and when a stop codon was introduced in place of MAAP-Q6, while using a MAAP-flag tag fusion protein.

We further characterized the MAAP start codon by comparing the size of the wild-type MAAP with recombinant MAAP in which we changed the MAAP-L1 CTG start codon to an ATG, or when N-terminally truncated MAAP was expressed from the second potential start codon (MAAP-R13, AGG) changed to ATG. We detected the MAAP of the same molecular weight as the protein expressed from MAAP-L1 modified to ATG, while recombinant N-terminal truncated MAAP expressed from MAAP-R13 modified to ATG is detected at lower molecular weight in Western blot.

We also produced MAAP with enhanced green fluorescent protein (eGFP) fused in its C-terminal part (MAAP-GFP), and cloned it into the wt-AAV2 genome. It results in functional disruption of VP1/2 proteins. This is due to the insertion of the eGFP in the cap ORF frame. However, the capacity to encode the AAP, and VP3 proteins should be conserved. Likewise, the Rep protein expression and regulation of the p40 promoter should not be impaired. Thus, detected fluorescence originating from eGFP should reflect the production level of the MAAP protein in the viral context.

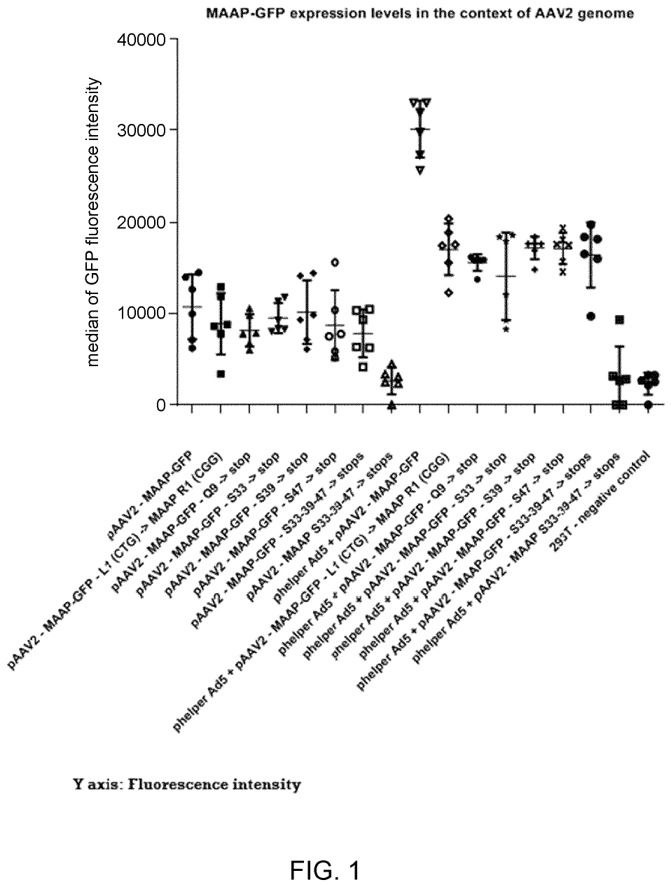

The MAAP-GFP fusion protein expressed from the wt-AAV2 genome had a median fluorescence intensity of 30872 when co-transfected with the plasmid encoding Adenovirus 5 helper genes (FIG. 1). The mutation of the MAAP-L1 CTG start to a CGG codon led to a fluorescent intensity of 17524. See FIG. 1. That is similar to the intensity level detected when we introduced a stop codon in place of MAAP-Q39 or MAAP-S47 or simultaneously to all three positions MAAP-S33,-39 and -47. When we introduced a stop codon in place of MAAP-Q9 or MAAP-S33, the levels of fluorescence intensity fell to 15896 and 15021, respectively. This level of expression remains above the background level and could hint at potential initiation of translation at different positions in the MAAP protein when stop codons are introduced in the reading frame. Alternatively, this level of expression could hint at potential read through of the inserted stop codons. We note, however, that with these new stop codons inserted, we were not able to detect any MAAP production at all using Western blot.

In the absence of adenovirus helper plasmid, the production level of MAAP-GFP was detected above the background level. This may be because the HEK293T cell line includes a copy of the adenoviral E1 gene. That E1 gene may act as trans-activator for AAV promoters.

Our experiments confirm the MAAP-L1 (CTG) as the start codon of the wild-type MAAP. Downstream of MAAP-L1, potential start codons located at position MAAP-R13 (AGG), MAAP-T14 (ACG) or further downstream of the MAAP protein may be used to translate an N-terminally truncated version of the MAAP, when translation of MAAP from L1 is impaired, as reflected by the MAAP-GFP production results.

### Kinetic of MAAP Production

MAAP is expressed from the cap gene, possibly from the spliced form of the p40 transcript leading to VP2/3 expression. According to ribosome scanning mechanism, we find that translation initiates at the CTG start codon of the MAAP protein (frame-shifted +1 to VP1 orf), followed by VP2 translation initiated at the ACG start codon, continued with AAP expression at a CTG codon (frame-shifted +1 to VP1 orf), and achieved by the VP3 protein, initiated at an ATG codon.

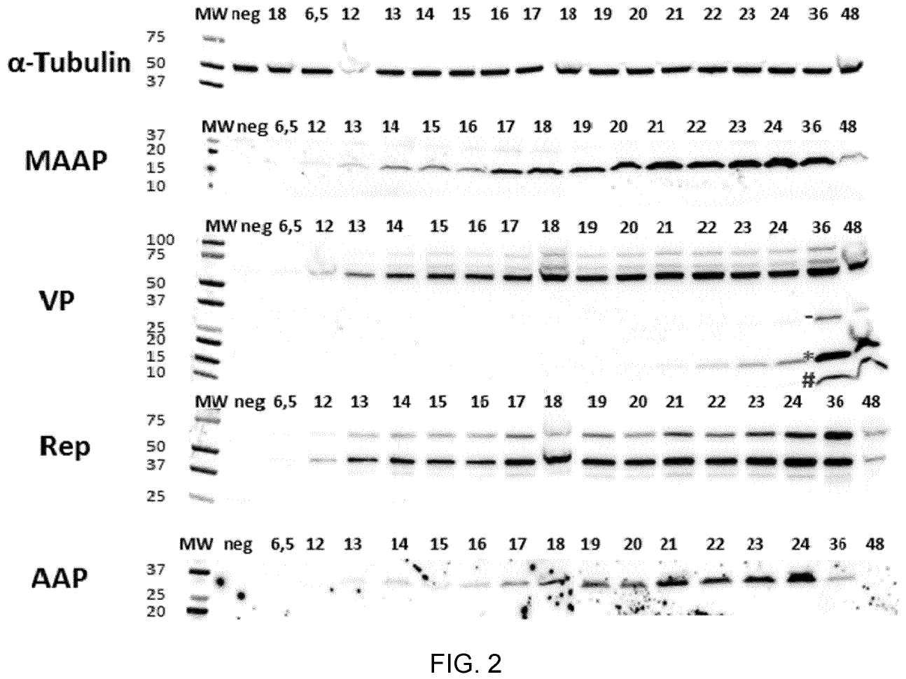

In a kinetic experiment, we followed the expression over time of the Rep78/52, VPs, AAP and MAAP proteins during WT AAV2 production in 293T cells. At 6.5 hours after transfection, we detected the very faint expression of VP3 and Rep52. At 12 hours post-transfection, we detected all AAV proteins except AAP. At 13 hours post-transfection, we detected AAP. See FIG. 2. Additionally, Protein expression intensified progressively and reached a plateau 21 h post-transfection. During AAV production, we could only detect the Rep78 and 52 isoform but not Rep 68 and 40.

During the production process, we also observed capsid degradation starting 21 h post-transfection. This is seen as lower than VP3 protein bands on Western blot using Progen B1 antibody targeting the C-terminal part of VPs.—See FIG. 2.

The AAP C-terminal region displays nuclear and nucleolar localization signal composed of five basic amino acid rich (“BR”) clusters. Any combination of 4 of these BR clusters will target the protein to the nucleus and nucleolus.

Similarly, we found that the MAAP C-terminal end displays three BR clusters: KKIR (BR1), RRKR (BR2), and RNLLRRLREKRGR (BR3). These are shown on SEQ ID NO. 2 at residues 78-82, 94-97 and 107-119 respectively. We thus concluded that the C-terminal part of the MAAP protein may contain nuclear localization signal(s).

### Effect of MAAP on Wild Type AAV

MAAP Inactivation and Impact on AAV Production

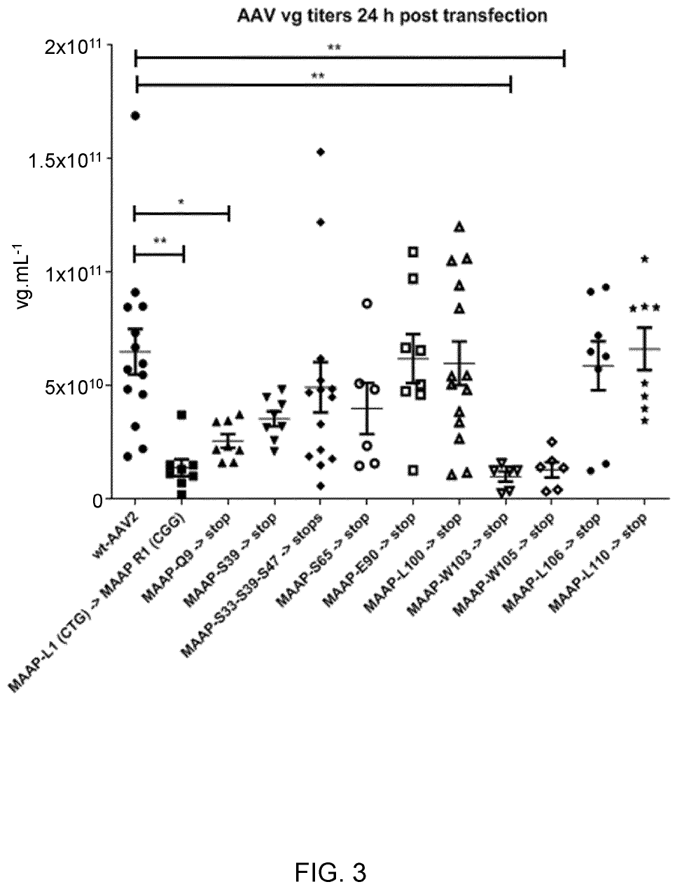

The modification of MAAP, either by the mutation of the start codon or introduction of stop codons at various positions in the MAAP coding sequence led to a reduction of AAV productivity at 24 h post transfection. See FIG. 3. This reduction in AAV productivity was statistically significant when MAAP-L1 (CTG) was modified to MAAP-R1 (CGG), with a productivity decreasing to only 21% of that observed with wt-AAV2. When a stop codon was introduced in place of MAAP-Q9, productivity decreased to only 39% of that observed with wt-AAV2. We also observed a reduction to 15% and 20% of wild-type productivity (respectively) for AAV mutants for which MAAP-W103 or MAAP-W105 were mutated to stop codon. Introduction of stop codons in place of MAAP-S33-39-S47, MAAP-S65, led to a trend in the reduction of AAV titers to 76% and 61% of levels seen with compared to wt-AAV2. Introduction of a stop codon in place of amino acid residue number 90, 100, 106 or 110 produced no clear trend in difference viz results observed with the wild-type gene. See FIG. 3. Thus, 24 h post transfection the expression of MAAP protein provides a replicative advantage compared to AAV mutants in which the MAAP gene is inactivated. Our results differ from Ogden (2019), who observed that the MAAP mutants did not have reduced titers. However, they observed that MAAP mutants were out-competed unless complemented in trans with functional MAAP polypeptide. This conclusion fits with our observed results of MAAP providing a replicative advantage to wt-AAV2 at 24 h post transfection.

TABLE 6
Amount of Viral Genomes
Amount of Viral Genomes (υg)/mL

| Rep | | | | | | | | | | | | |
|---|---|---|---|---|---|---|---|---|---|---|---|---|
| 24 h | A | B | C | D | E | F | G | H | I | J | K | L |
| I | 6.49 | 1.38 | 2.55 | 3.53 | 4.93 | 3.99 | 6.18 | 5.98 | 0.99 | 1.28 | 5.87 | 6.62 |
| II | — | 0.21 | 0.39 | 0.54 | 0.76 | 0.61 | 0.95 | 0.92 | 0.15 | 0.20 | 0.90 | 1.02 |
| n | 7 | 4 | 4 | 4 | 7 | 3 | 4 | 7 | 3 | 3 | 4 | 1 |

KEY:
Column A = wt-AAV2.
B = MAAP-L1 (CTG) → MAAP R1 (CGG).
C = MAAP-Q9 → stop.
D = MAAP-S39 → stop.
E = MAAP-S33-S39-S47 → stop.
F = MAAP-S65 → stops.
G = MAAP-E90 → stop.
H = MAAP-L100 → stop.
I = MAAP-W103 → stop.
J = MAAP-W105 → stop.
K = MAAP-L106 → stop.
L = MAAP-L110 → stop.
Row I = mean (vg.mL⁻¹) × 10¹⁰.
Row II = fold difference vs wt-AAV.
n = number of samples.

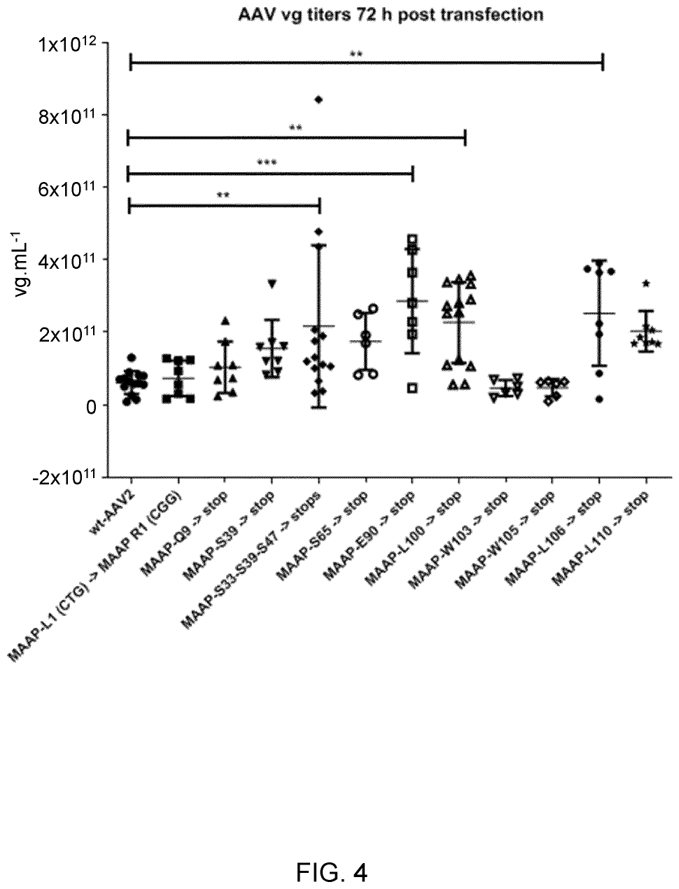

At 72 h time point, only MAAP mutants in which MAAP-W103 and MAAP-W105 were replaced by stop codons showed reduced titers, respectively 0.75 and 0.76-fold, compared to wt-AAV2. See FIG. 4. Surprisingly, however, we found that for all other studied MAAP mutants, AAV titers were improved, rather than decreased. This surprising increase in AAV titer was significant for mutants in which MAAP-S33-S39-S47, MAAP-E90, MAAP-L100, MAAP-L106, are replaced by stop codons, with titers respectively increased by 3.50, 4.62, 3.67, 4.08-fold compared to wt-AAV2. Thus, when production time (i.e., the time transfected cells are cultured to produce virus) is extended beyond 24 hours, inactivation of MAAP protein results in higher AAV titers than does wild-type AAV.

TABLE 7
Amount of Viral Genomes
Amount of Viral Genomes (υg)/mL

| Rep | | | | | | | | | | | | |
|---|---|---|---|---|---|---|---|---|---|---|---|---|
| 72 h | A | B | C | D | E | F | G | H | I | J | K | L |
| I | 6.19 | 7.35 | 10.3 | 15.5 | 21.7 | 17.4 | 28.6 | 22.7 | 4.62 | 4.74 | 25.3 | 20.3 |
| II | — | 1.19 | 1.67 | 2.51 | 3.50 | 2.82 | 4.62 | 3.67 | 0.75 | 0.76 | 4.08 | 3.28 |
| n | 7 | 4 | 4 | 4 | 7 | 3 | 4 | 7 | 3 | 3 | 4 | 4 |

KEY:
Column A = wt-AAV2.
Column B = MAAP-L1 (CTG) → MAAP R1 (CGG).
Column C = MAAP-Q9 → stop.
Column D = MAAP-S39 → stop.
Column E = MAAP- S33-S39-S47 → stop.
Column F = MAAP-S65 → stops.
Column G = MAAP-E90 → stop.
Column H = MAAP-L100 → stop.
Column I = MAAP-W103 → stop.
Column J = MAAP-W105 → stop.
Column K = MAAP-L106 → stop.
Column L =MAAP-L110 → stop.
Row I = mean (vg.mL⁻¹) × 10¹⁰.
Row II = fold difference vs wt-AAV2.
n = number of samples.

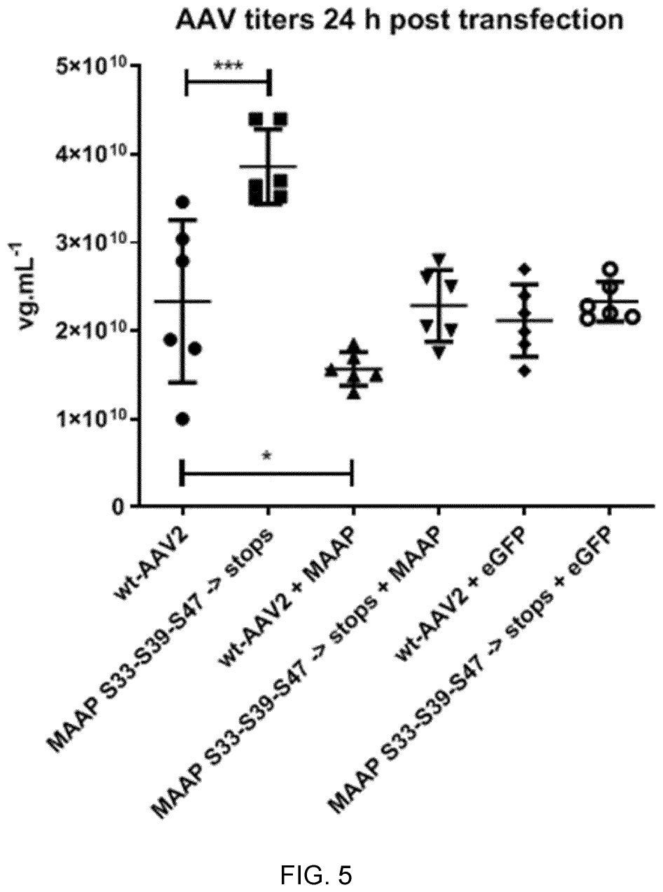

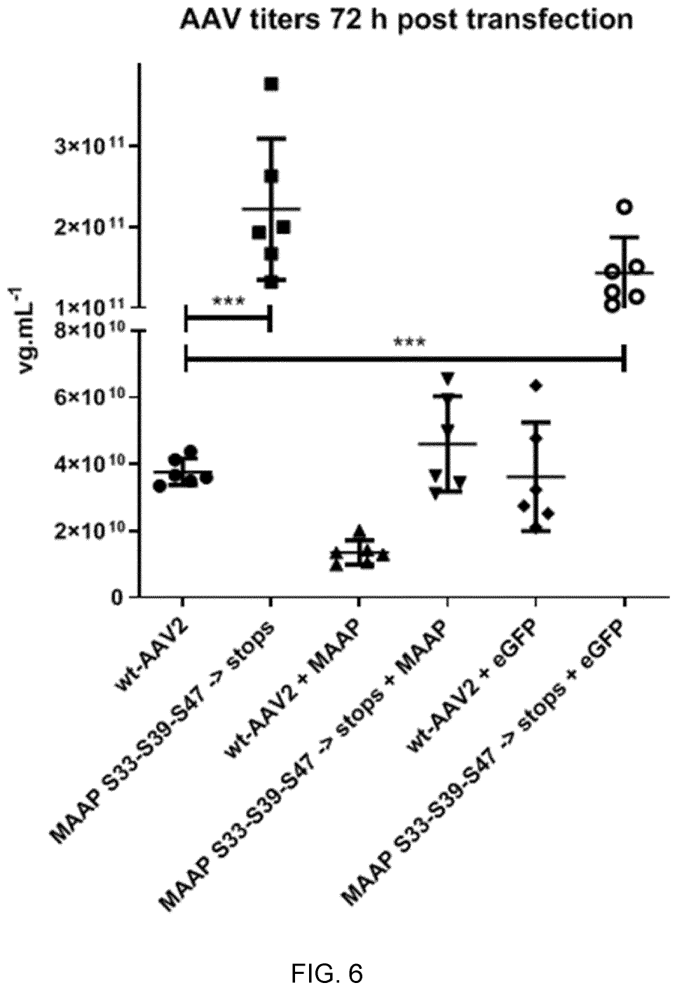

Next, we studied the effect of MAAP over-production to wt-AAV production, or to AAV mutant where MAAP-S33-S39-S47 were mutated to stop codons. At 24 h after transfection, when additional MAAP was expressed with wt-AAV2, we observed a 0.67-fold reduction in AAV titer compared to wt-AAV2. See FIG. 5, left. At 72 h time point, a 0.36-fold reduction compared to wt-AAV2 was observed. See FIG. 6, right. When we added to the wt-AAV reference cell a plasmid of similar size to the MAAP expression plasmid (to mimic the additional DNA mass provided by the addition of the MAAP expression plasmid), a 0.91-fold reduction compared to wt-AAV2 reference was observed at 24 h time point and 0.96-fold reduction at 72 h time point.

TABLE 8
Amount of Viral Genomes
Amount of Viral Genomes (υg)/mL

| Rep 24 h | A A | B | C | D | E | F |
|---|---|---|---|---|---|---|
| I | 2.33 | 3.86 | 1.57 | 2.28 | 2.12 | 2.33 |
| II | — | 1.66 | 0.67 | 0.98 | 0.91 | 1.00 |

KEY:
Column A = wt-AAV2.
Column B = MAAP S33-S39-S47 → stops.
Column C = wt-AAV1 + MAAP.
Column D = MAAP S33-S39-S47 → stops + MAAP.
Column E = wt-AAV2 + eGFP.
Column F = MAAP S33-S39-S47 → stops + eGFP.
Row I = mean (vg.mL⁻¹) × 10¹⁰.
Row II = fold difference vs wt-AAV2.

TABLE 9
Amount of Viral Genomes
Amount of Viral Genomes (υg)/mL

| Rep 72 h | A | B | C | D | E | F |
|---|---|---|---|---|---|---|
| I | 3.77 | 22.2 | 1.36 | 4.61 | 3.63 | 14.3 |
| II | — | 5.89 | 0.36 | 1.22 | 0.96 | 3.80 |

KEY:
Column A = wt-AAV2.
Column B = MAAP S33-S39-S47 → stops.
Column C = wt-AAV1 + MAAP.
Column D = MAAP S33-S39-S47 → stops + MAAP.
Column E = wt-AAV2 + eGFP.
Column F = MAAP S33-S39-S47 → stops + eGFP.
Row I = mean (vg.mL⁻¹) × 10¹⁰.
Row II = fold difference vs wt-AAV2.

When we produced AAV for which MAAP-S33-S39-S47 are mutated to stop codons we observed a 1.66 fold increase in vg titers compared to wt-AAV2 at 24 h time point and 5.89-fold increase at 72 h time point.

MAAP added to AAV for which MAAP-S33-S39-S47 are mutated to stop codons resulted in vg titers similar to wt-AAV2 at 24 h time point and with 1.22-fold increase at 72 h time point. When the MAAP-S33-S39-S47 are mutated to stop codons and is complemented with plasmid of similar size to the MAAP expression plasmid, the titers were equal to the wt-AAV2 reference at 24 h time point and 3.80-fold higher at 72 h time point.

In summary, for culture periods longer than 24 hours, we surprisingly found that the over-expression of the MAAP protein resulted in reduced viral genome (vg) titers. This suggests that MAAP is required in a stoichiometric amount relative to another AAV protein(s), potentially the AAP or the VPs.

Packaging of Contaminating DNA

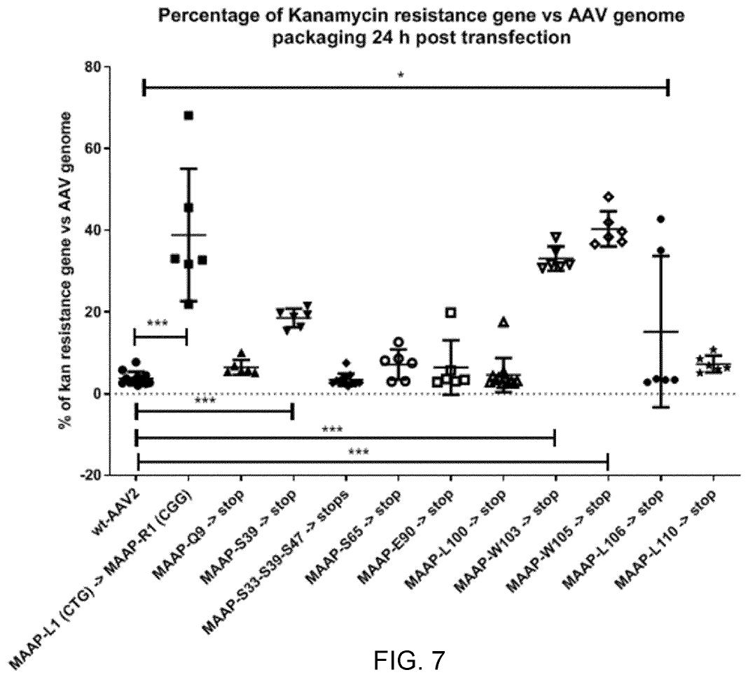

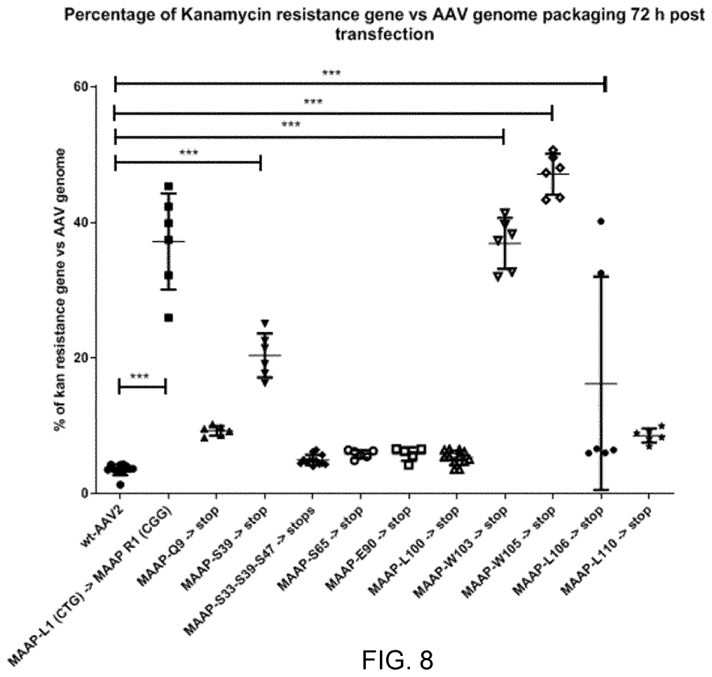

Next, we studied the effect of MAAP on the packaging of contaminating DNA, into the AAV capsid, originating from the AAV producer plasmids. We first studied the level of kanamycin-resistance gene packaged in the AAV capsid by ddPCR. The kanamycin-resistance gene is present both in the adenovirus serotype 5 helper plasmid and the plasmid encoding the AAV genome. During wt-AAV production, at 24 h after transfection, we measured 3.77% of Kanamycin resistance gene contamination packaged, compared to wt-AAV2 genome packaging, while at 72 h time point this percentage was equal to 3.50%. See FIGS. 7 and 8.

TABLE 10
Kanamycin resistance gene vs AAV genome packaging
% of Kanamycin resistance gene vs AAV genome packaging
at 24 hours

| | A | B | C | D | E | F | G | H | I | J | K | L |
|---|---|---|---|---|---|---|---|---|---|---|---|---|
| I | 3.77 | 38.88 | 6.47 | 18.55 | 3.47 | 7.20 | 6.45 | 4.59 | 33.10 | 40.34 | 15.19 | 7.29 |
| II | — | 10.31 | 1.72 | 4.92 | 0.92 | 1.91 | 1.71 | 1.22 | 8.78 | 10.70 | 4.03 | 1.93 |
| n | 6 | 3 | 3 | 3 | 6 | 3 | 3 | 6 | 3 | 3 | 3 | 3 |

KEY:
Column A = wt-AAV2.
Column B = MAAP-L1 (CTG) → MAAP R1 (CGG).
Column C = MAAP-Q9 → stop.
Column D = MAAP-S39 → stop.
Column E = MAAP-S33-S39-S47 → stop.
Column F = MAAP-S65 → stops.
Column G = MAAP-E90 → stop.
Column H = MAAP-L100 → stop.
Column I = MAAP-W103 → stop.
Column J = MAAP-W105 → stop.
Column K = MAAP-L106 → stop.
Column L = MAAP-L110 → stop.
Row I = % of Kanamycin resistance gene vs AAV genome packaging.
Row II = fold difference vs wt-AAV2.
n = number of samples.

TABLE 11
Kanamycin resistance gene vs AAV genome packaging
% of Kanamycin resistance gene vs AAV genome packaging
at 72 hours

| | A | B | C | D | E | F | G | H | I | J | K | L |
|---|---|---|---|---|---|---|---|---|---|---|---|---|
| I | 3.50 | 37.21 | 9.25 | 20.40 | 4.93 | 5.82 | 5.82 | 5.27 | 36.93 | 47.12 | 16.27 | 8.55 |
| II | — | 10.63 | 2.64 | 5.83 | 1.41 | 1.66 | 1.66 | 1.51 | 10.55 | 13.46 | 4.65 | 2.44 |
| n | 6 | 3 | 3 | 3 | 6 | 3 | 3 | 6 | 3 | 3 | 3 | 3 |

KEY:
Column A = wt-AAV2.
Column B = MAAP-L1 (CTG) → MAAP R1 (CGG).
Column C = MAAP-Q9 → stop.
Column D = MAAP-S39 → stop.
Column E = MAAP-S33-S39-S47 → stop.
Column F = MAAP-S65 → stops.
Column G = MAAP-E90 → stop.
Column H = MAAP-L100 → stop.
Column I = MAAP-W103 → stop.
Column J = MAAP-W105 → stop.
Column K = MAAP-L106 → stop.
Column L = MAAP-L110 → stop.
Row I = % of Kanamycin resistance gene vs AAV genome packaging.
Row II = fold difference vs wt-AAV2.
n = number of samples.

TABLE 12
Kanamycin resistance gene vs AAV genome packaging.
% of Kanamycin resistance gene us AAV genome packaging
at 24 hours

| | A | B | C | D | E | F |
|---|---|---|---|---|---|---|
| I | 4.05 | 3.11 | 7.08 | 7.34 | 5.43 | 5.09 |
| II | — | 0.77 | 1.75 | 1.81 | 1.34 | 1.26 |

KEY:
Column A = wt-AAV2.
Column B = MAAP S33-S39-S47 → stops.
Column C = wt-AAV2 + MAAP.
Column D = MAAP S33-S39-S47 → stops + MAAP.
Column E = wt-AAVS + eGFP.
Column F = MAAP S33-39-S47 → stops + eGFP.
Row I = % of Kanamycin resistance gene vs AAV genome packaging.
Row II = fold difference vs wt-AAV2.

TABLE 13
Kanamycin resistance gene vs AAV genome packaging.
of Kanamycin resistance gene vs AAV genome packaging
at 72 hours

| | A | B | C | D | E | F |
|---|---|---|---|---|---|---|
| I | 5.78 | 6.10 | 10.15 | 8.43 | 5.22 | 5.86 |
| II | — | 1.06 | 1.76 | 1.46 | 0.90 | 1.01 |

KEY:
Column A = wt-AAV2.
Column B = MAAP S33-S39-S47 → stops.
Column C = wt-AAV2 + MAAP.
Column D = MAAP S33-S39-S47 → stops + MAAP.
Column E = wt-AAVS + eGFP.
Column F = MAAP S33-39-S47 → stops + eGFP.
Row I = % of Kanamycin resistance gene vs AAV genome packaging.
Row II = fold difference vs wt-AAV2.

The level of kanamycin gene packaging was increased significantly when MAAP-L1 CTG start codon was modified to CGG, or when MAAP-S39, MAAP-W103 or MAAP-W105 and MAAP-L106 were modified to stop codon, with increases respectively of 10.31, 4.92, 8.78, 10.70 and 4.03-fold compared to wt-AAV at 24 h time point. See FIG. 7. This increased 10.63, 5.83, 10.55, 13.46 and 4.65-fold compared to wt-AAV at 72 h time point. See FIG. 8. The highest contamination level was observed for the MAAP-W105 mutated to stop codon with an increase of 13.46-fold over wt-AAV, with Kanamycin resistance gene packaging representing 47.12% compared to the AAV genome. See FIG. 8.

AAV for which MAAP-S33-S39-S47 are mutated to stop codons, MAAP-S65, MAAP-E90, MAAP-L100 and MAAP-L110 showed a trend of higher Kanamycin packaging compared to wt-AAV2 both at 24 h and at 72 h time points.

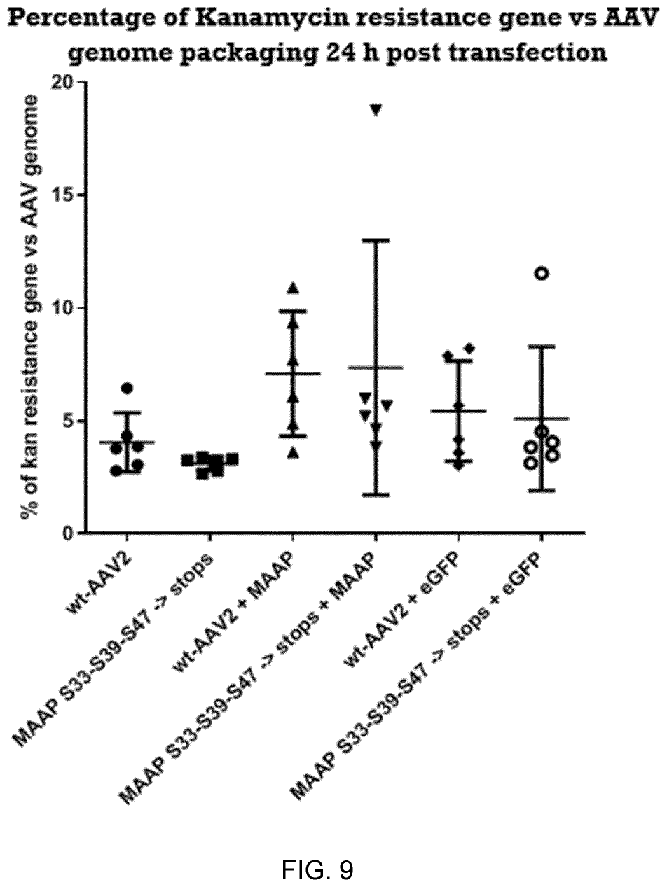

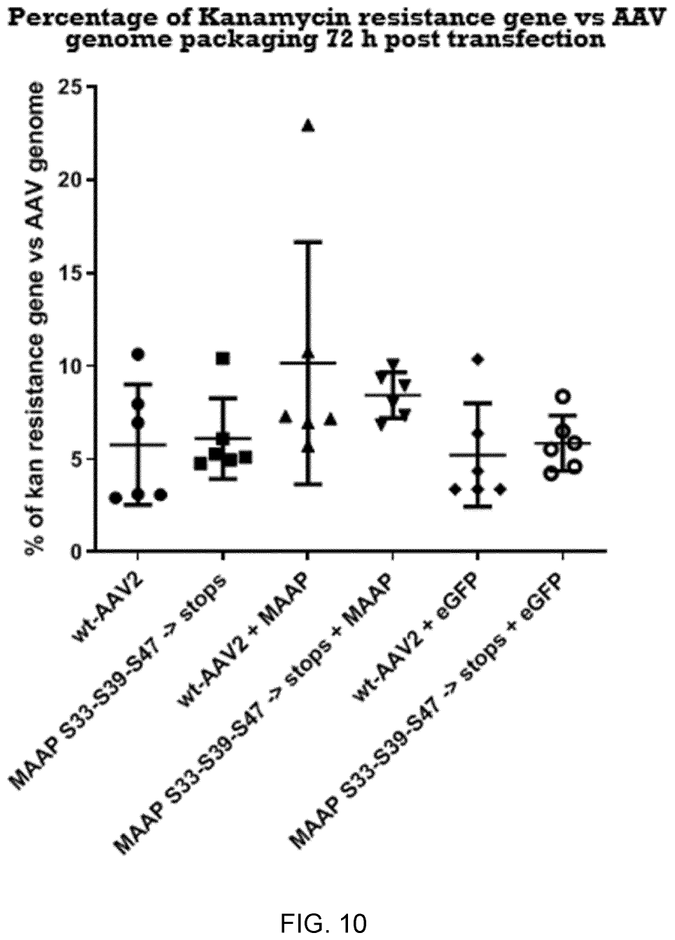

When MAAP was added in complement to wt-AAV production or to AAV in which MAAP-S33-S39-S47 are mutated to stop codons, we observed an increase in Kanamycin resistance gene packaging compared to the wt-AAV without addition MAAP protein expression. See FIGS. 9 and 10.

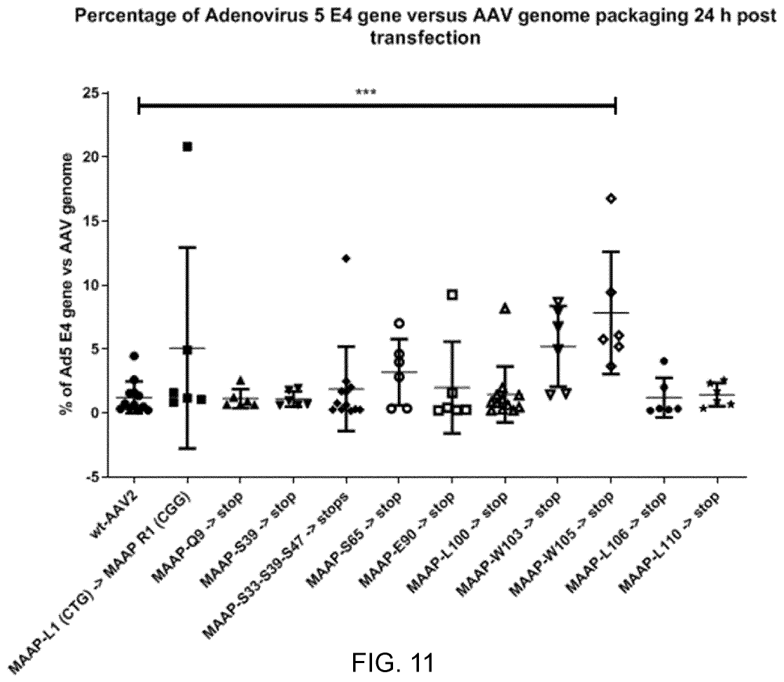

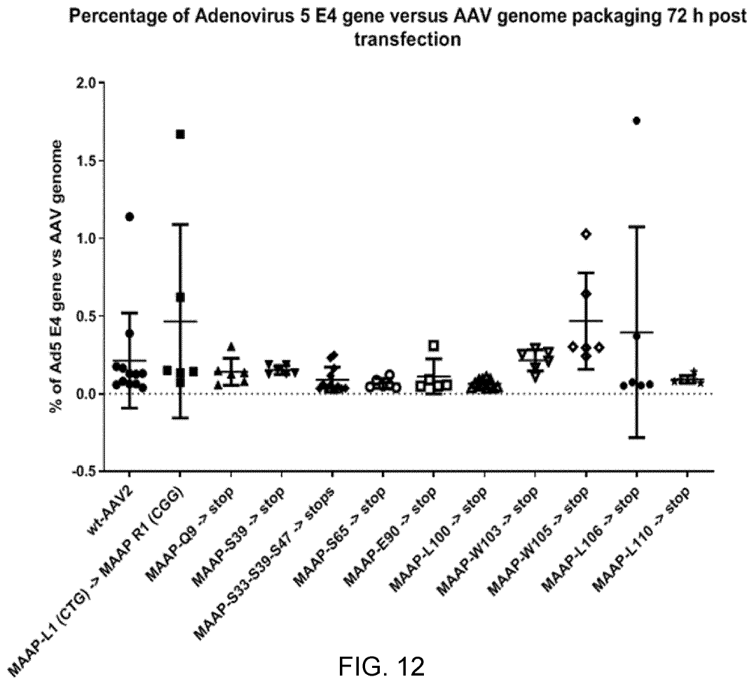

To study whether the antibiotic resistance gene contamination is originating preferentially from the Adenovirus helper plasmid or from the wt-AAV2 encoding plasmid, we measured the level of contamination originating from the Adenovirus 5 E4 gene present on the plasmid encoding the Adenovirus 5 helper functions necessary for AAV production. When MAAP-L1 CTG start codon was modified to CGG, we observed statistically significant increase only in Adenovirus 5 E4 gene packaging of 6.37-fold at 24 h time point. See FIG. 11. At 72 h time point, no statistically significant differences compared to wt-AAV2 were detected. However, at 72 h time point, a trend in reduction of Adenovirus 5 E4 gene packaging was observed particularly for mutants in which stop codons are introduced in place of MAAP-S33-S39-S47, MAAP-S65, MAAP-E90, MAAP-L100 or MAAP-L110 with 0.43, 0.32, 0.53, 0.31, 0.44-fold respectively. See FIG. 12.

TABLE 14
Ad5 E4 gene vs AAV genome packaging.
% of Ad5 E4 gene vs AAV genome packaging
at 24 hours

| | A | B | C | D | E | F | G | H | I | J | K | L |
|---|---|---|---|---|---|---|---|---|---|---|---|---|
| 1 | 1.23 | 5.09 | 1.14 | 1.12 | 1.90 | 3.21 | 2.00 | 1.47 | 5.23 | 7.83 | 1.22 | 1.45 |
| II | | 4.13 | 0.92 | 0.91 | 1.55 | 2.61 | 1.63 | 1.19 | 4.25 | 6.37 | 0.99 | 1.18 |
| n | 6 | 3 | 3 | 3 | 6 | 3 | 3 | 6 | 3 | 3 | 3 | 3 |

KEY:
Column A = wt-AAV2.
Column B = MAAP-L1 (CTG) → MAAP R1 (CGG).
Column C = MAAP-Q9 → stop.
Column D = MAAP-S39 → stop.
Column E = MAAP-S33-S39-S47 → stop.
Column F = MAAP-S65 → stops.
Column G = MAAP-E90 → stop.
Column H = MAAP-L100 → stop.
Column I = MAAP-W103 → stop.
Column J = MAAP-W105 → stop.
Column K = MAAP-L106 → stop.
Column L = MAAP-L110 → stop.
Row I = % of Ad5 E4 gene vs AAV genome packaging.
Row II = fold difference vs wt-AAV2.
n = number of samples.

TABLE 15
Ad5 E4 gene vs AAV genome packaging.
% of Ad5 E4 gene vs AAV genome packaging
at 72 hours

| | A | B | C | D | E | F | G | H | I | J | K | L |
|---|---|---|---|---|---|---|---|---|---|---|---|---|
| I | 0.21 | 0.47 | 0.14 | 0.15 | 0.09 | 0.07 | 0.11 | 0.06 | 0.22 | 0.47 | 0.39 | 0.09 |
| II | — | 2.22 | 0.68 | 0.73 | 0.43 | 0.32 | 0.53 | 0.31 | 1.03 | 2.23 | 1.88 | 0.44 |
| n | 6 | 3 | 3 | 3 | 6 | 3 | 3 | 6 | 3 | 3 | 3 | 3 |

KEY:
Column A = wt-AAV2.
Column B = MAAP-L1 (CTG) → MAAP R1 (CGG).
Column C = MAAP-Q9 → stop.
Column D = MAAP-S39 → stop.
Column E = MAAP-S33-S39-S47 → stop.
Column F = MAAP-S65 → stops.
Column G = MAAP-E90 → stop.
Column H = MAAP-L100 → stop.
Column I = MAAP-W103 → stop.
Column J = MAAP-W105 → stop.
Column K = MAAP-L106 → stop.
Column L = MAAP-L110 → stop.
Row I = % of Ad5 E4 gene vs AAV genome packaging.
Row II = fold difference vs wt-AAV2.
n = number of samples.

The results suggest that a higher level of contaminating DNA is packaged in the AAV capsid when MAAP expression is impaired, or when it originates from the same plasmid backbone that encodes the AAV genome. However, DNA packaged from the adenovirus helper plasmid is reduced compared to the wt-AAV, particularly where MAAP-S33-S39-S47, MAAP-S65, MAAP-E90, MAAP-L100 or MAAP-L110 have been mutated to a stop codon. Overall, our results indicate that MAAP may be involved in ITR-mediated DNA packaging independently of the ITR D sequence, present on the AAV genome side but absent on the backbone side of the ITRs.

MAAP Impact on AAV Capsid

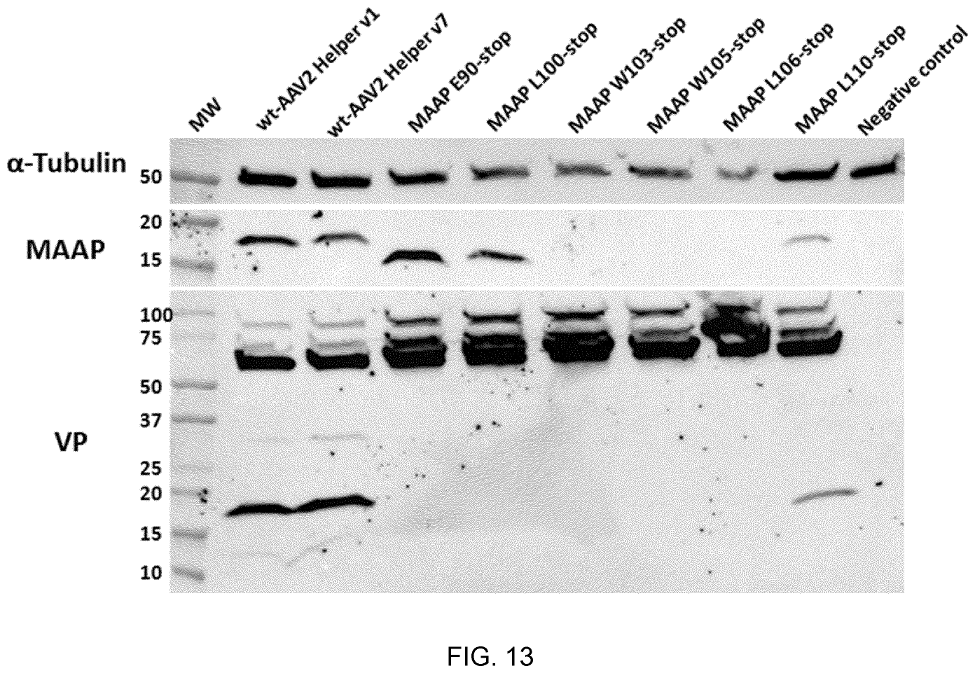

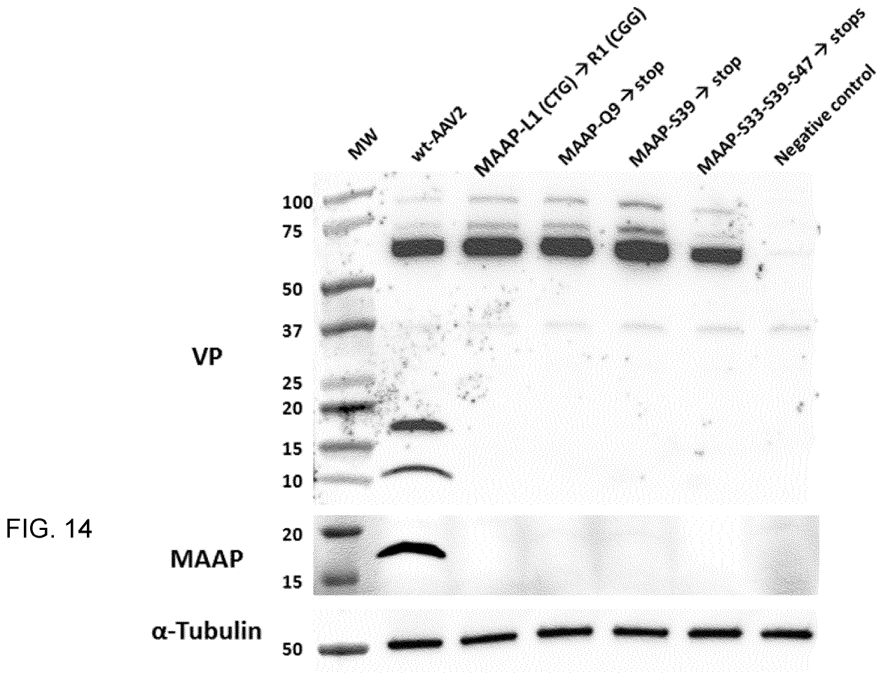

The wild-type AAV capsid is composed of the VP1, VP2 and VP3 proteins in a relative ratio of about 1:1:10. However, specific degradation products of these VPs are detected starting 21 h post-transfection in 293 and 293T cells. See FIG. 2 For example, in AAV serotype 2, VP-1, -2 and -3 are about 87, 73, and 61 kDa respectively. We detected degradation of the VPs by using a monoclonal antibody against each of the C-terminal ends of the three VPs. (The antibodies are described in Wobus C E et al., Monoclonal Antibodies against the Adeno-Associated Virus Type 2(AAV-2) Capsid: Epitope Mapping and Identification of Capsid Domains Involved in AAV-2-Cell Interaction and Neutralization of AAV-2Infection, 74 J. Virol. 9281 (2000).) Using this, we observed VP1, VP2 and VP3 degrading within 21 hours after transfection. These are observed in FIG. 2 at at 32 kDa (FIG. 2 dash; FIG. 13; FIG. 14), 18 kDa (FIG. 2 asterisk; FIG. 13; FIG. 14), and 14 kDa (FIG. 2 hash; FIG. 13; FIG. 14).

Interestingly, for all constructs in which MAAP expression was inactivated, except the MAAP-L110 to stop mutant, we observed the disappearance of the three 32 kDa, 18 kDa and 14 kDa polypeptides (FIG. 13; FIG. 14). We also observed an increase in the quantity of VP1 and VP2 proteins. This suggests that the degradation products originated from the degradation of VP1 and VP2 specifically (FIG. 13; FIG. 14). However, the variant in which MAAP-L110 was mutated to stop codon still displayed the specific 18 kDa AAV capsid degradation product, but with lower intensity compared to wt-AAV2. This suggests that the specific proteolytic activity observed, when MAAP is expressed, is associated with its C-terminal part, more specifically with the basic amino acid-rich “BR3” domain (amino acid residues 107-119 on SEQ ID NO. 2), as only the MAAP-L110 to stop mutant, still encoding the BR3 domain, displayed the VP proteolytic fragment. When the MAAP was expressed in complement to the AAV mutant in which MAAP-S33-S39-S47 are replaced by stop codons, the proteolytic activity on the AAV capsid was restored. These data show that the virus preparation made from a genome that is MAAP-deleted or MAAP-truncated to eliminate the proteolytic domain, is physically different from virus made from a wild-type genome. Our finding here thus provides a way to increase both the yield of AAV, and the stability of the resulting AAV.

We analyzed the ratio of capsids containing the AAV genome in comparison to total AAV capsids at 72 h, to derive the ratio of empty capsids (lacking the desired DNA) to full capsids (carrying the desired DNA). Wild-type AAV2 showed 6.91% of capsid containing the AAV genome.

TABLE 16
Total Capsids Containing AAV Genome.
Percent of Total Capsids Containing AAV Genome

| | I | II | III | IV | V | VI | VII | VIII | IX | X | XI | XII |
|---|---|---|---|---|---|---|---|---|---|---|---|---|
| % | 6.91 | 3.39 | 4.60 | 5.99 | 11.80 | 13.07 | 8.24 | 8.76 | 4.52 | 3.71 | 7.04 | 7.04 |
| vs | = | 0.49 | 0.67 | 0.87 | 1.71 | 1.89 | 1.19 | 1.27 | 0.65 | 0.54 | 1.02 | 1.02 |
| wt | | | | | | | | | | | | |

Column Number Key:
I = wt-AAV2
II = MAAP gene with start codon (CTG) (L1 on the full-length polypeptide) mutated to CGG (R1)
III= MAAP gene with Q9 on the full-length polypeptide point-mutated to stop codon.
IV = MAAP gene with S39 point-mutated to stop codon.
V = MAAP gene with all of S33, S39 and S47 point-mutated to stop codon.
VI to XII = MAAP gene with S65, E90, L100, L103, L105, L106 or L110 (respectively) point-mutated to stop codon.

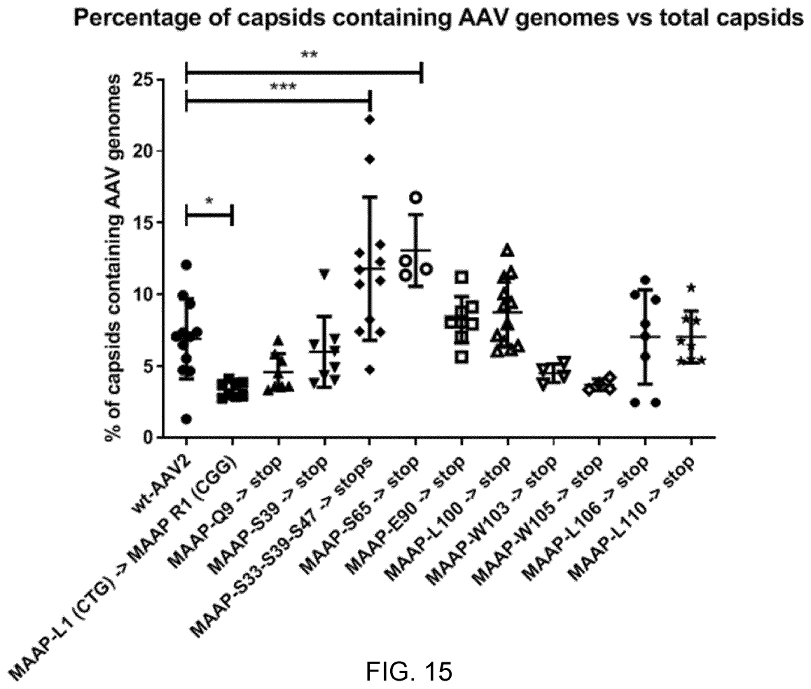

A reduction of capsids containing genome was observed when MAAP-L1 CTG start codon was modified to CGG, or when AAV MAAP-Q9, MAAP-39, MAAPW103 or MAAP-W105 were replaced by a stop codon. AAV MAAP-L106 or MAAP-L11=mutated to stop, encoding almost full-length MAAP, had similar level of AAV capsids containing AAV genomes compared to wt-AAV2. When AAV MAAP-S33-S39-S47 were replaced to stops, or when MAAP-S65, MAAP-E90 and MAAP-L100 were mutated to stop codon, we observed an increase of capsids containing AAV genomes compared to wt-AAV2. We plot more detailed data in FIG. 15.

TABLE 17
Capsids containing AAV genome
% of capsids containing AAV genome

| | A | B | C | D | E | F | G | H | I | J | K | L |
|---|---|---|---|---|---|---|---|---|---|---|---|---|
| I | 6.91 | 3.39 | 4.60 | 5.99 | 11.80 | 13.07 | 8.24 | 8.76 | 4.52 | 3.71 | 7.04 | 7.04 |
| II | — | 0.49 | 0.67 | 0.87 | 1.71 | 1.89 | 1.19 | 1.27 | 0.65 | 0.54 | 1.02 | 1.02 |

MAAP Truncation
Effect on Virus Yield and Purity

| | | | | | | | | | | | | |
|---|---|---|---|---|---|---|---|---|---|---|---|---|
| MAAP | | | | | | | | | | | | |
| variant | I | II | III | IV | V | VI | VII | VIII | IX | X | XI | XII |

Rep 24 h

| | | | | | | | | | | | | |
|---|---|---|---|---|---|---|---|---|---|---|---|---|
| mean | 6.49 | 1.38 | 2.55 | 3.53 | 4.93 | 3.99 | 6.18 | 5.98 | 0.99 | 1.28 | 5.87 | 6.62 |
| (vg/mL) * | | | | | | | | | | | | |
| 10¹⁰ | | | | | | | | | | | | |
| fold | — | 0.21 | 0.39 | 0.54 | 0.76 | 0.61 | 0.95 | 0.92 | 0.15 | 0.20 | 0.90 | 1.02 |
| difference vs | | | | | | | | | | | | |
| wt-AAV2 | | | | | | | | | | | | |
| N | 14 | 8 | 8 | 8 | 14 | 6 | 8 | 14 | 6 | 6 | 8 | 8 |

108

Rep 72 h

| | | | | | | | | | | | | |
|---|---|---|---|---|---|---|---|---|---|---|---|---|
| mean | 6.19 | 7.35 | 10.3 | 15.5 | 21.7 | 17.4 | 28.6 | 22.7 | 4.62 | 4.74 | 25.3 | 20.3 |
| (vg/mL) * | | | | | | | | | | | | |
| 1010 | | | | | | | | | | | | |
| fold | — | 1.19 | 1.67 | 2.51 | 3.50 | 2.82 | 4.62 | 3.67 | 0.75 | 0.76 | 4.08 | 3.28 |
| difference vs | | | | | | | | | | | | |
| wt-AAV2 | | | | | | | | | | | | |
| N | 14 | 8 | 8 | 8 | 14 | 6 | 7 | 14 | 6 | 6 | 8 | 8 |

107

% Kan 24 h

| | | | | | | | | | | | | |
|---|---|---|---|---|---|---|---|---|---|---|---|---|
| % of kan vs | 3.77 | 38.9 | 6.47 | 18.6 | 3.47 | 7.20 | 6.45 | 4.59 | 33.1 | 40.3 | 15.2 | 7.29 |
| AAV genome | | | | | | | | | | | | |
| packaging | | | | | | | | | | | | |
| fold | — | 10.3 | 1.72 | 4.92 | 0.92 | 1.91 | 1.71 | 1.22 | 8.78 | 10.7 | 4.03 | 1.93 |
| difference vs | | | | | | | | | | | | |
| wt-AAV2 | | | | | | | | | | | | |
| N | 12 | 6 | 6 | 6 | 12 | 6 | 6 | 12 | 6 | 6 | 6 | 6 |

90

% Kan 72 h

| | | | | | | | | | | | | |
|---|---|---|---|---|---|---|---|---|---|---|---|---|
| % kan VS | 3.50 | 37.2 | 9.25 | 20.4 | 4.93 | 5.82 | 5.82 | 5.27 | 36.9 | 47.1 | 16.3 | 8.55 |
| AAV genome | | | | | | | | | | | | |
| packaging | | | | | | | | | | | | |
| fold | — | 10.6 | 2.64 | 5.83 | 1.41 | 1.66 | 1.66 | 1.51 | 10.6 | 13.5 | 4.65 | 2.44 |
| difference vs | | | | | | | | | | | | |
| wt-AAV2 | | | | | | | | | | | | |
| N | 12 | 6 | 6 | 6 | 12 | 6 | 5 | 12 | 6 | 6 | 6 | 6 |

89

% Ad5 E4 24 h

| | | | | | | | | | | | | |
|---|---|---|---|---|---|---|---|---|---|---|---|---|
| % of Ad5 E4 | 1.23 | 5.09 | 1.14 | 1.12 | 1.90 | 3.21 | 2.00 | 1.47 | 5.23 | 7.83 | 1.22 | 1.45 |
| gene vs AAV | | | | | | | | | | | | |
| genome | | | | | | | | | | | | |
| packaging | | | | | | | | | | | | |
| fold | — | 4.13 | 0.92 | 0.91 | 1.55 | 2.61 | 1.63 | 1.19 | 4.25 | 6.37 | 0.99 | 1.18 |
| difference vs | | | | | | | | | | | | |
| wt-AAV2 | | | | | | | | | | | | |
| N | 12 | 6 | 6 | 6 | 12 | 6 | 6 | 12 | 6 | 6 | 6 | 6 |

90

% Ad5 E4 72 h

| | | | | | | | | | | | | |
|---|---|---|---|---|---|---|---|---|---|---|---|---|
| % of Ad5 E4 | 0.21 | 0.47 | 0.14 | 0.15 | 0.09 | 0.07 | 0.11 | 0.06 | 0.22 | 0.47 | 0.39 | 0.09 |
| gene vs AAV | | | | | | | | | | | | |
| genome | | | | | | | | | | | | |
| packaging | | | | | | | | | | | | |
| fold | — | 2.22 | 0.68 | 0.73 | 0.43 | 0.32 | 0.53 | 0.31 | 1.03 | 2.23 | 1.88 | 0.44 |
| difference vs | | | | | | | | | | | | |
| wt-AAV2 | | | | | | | | | | | | |
| N | 12 | 6 | 6 | 6 | 12 | 6 | 5 | 12 | 6 | 6 | 6 | 6 |

89

% of capsids containing AAV genomes

| | | | | | | | | | |
|---|---|---|---|---|---|---|---|---|---|
| % | 7.20 | 3.13 | 3.77 | 4.56 | 9.45 | 8.00 | 6.83 | 5.80 | 5.99 |
| fold | — | 0.43 | 0.52 | 0.63 | 1.31 | 1.11 | 0.95 | 0.81 | 0.83 |
| difference vs | | | | | | | | | |
| wt-AAV2 | | | | | | | | | |

573.00
1719

KEY:
Column A = wt-AAV2.
Column B = MAAP-L1 (CTG) → MAAP R1 (CGG).
Column C = MAAP-Q9 → stop.
Column D = MAAP-S39 → stop.
Column E =MAAP-S33-S39-S47 → stop.
Column F = MAAP-S65 → stops.
Column G = MAAP-E90 → stop.
Column H = MAAP-L100 → stop.
Column I = MAAP-W103 → stop.
Column J = MAAP-W105 → stop.
Column K = MAAP-L106 → stop.
Column L = MAAP-L110 → stop.
Row I = %.
Row II = fold difference vs wt-AAV2.
n = number of samples.
Column Number Key:
I = wt-AAV2
II = MAAP gene with start codon (CTG) (L1 on the full-length polypeptide) mutated to CGG (R1)
III = MAAP gene with Q9 on the full-length polypeptide point-mutated to stop codon.
IV = MAAP gene with S39 point-mutated to stop codon.
V = MAAP gene with all of S33, S39 and S47 point-mutated to stop codon.
VI to XII = MAAP gene with S65, E90, L100, L103, L105, L106 or L110 (respectively) point-mutated to stop codon. N-values here are presented as all measured samples not taking account of actual independent experiments vs. above

### Conclusions

MAAP expression improves virus replication 24 hours after infection. Over longer infection times, however, we surprisingly found that MAAP expression deteriorates virus replication. We demonstrated that for infection periods greater than 24 hours, inactivating wild-type MAAP expression produces a higher yield of virus than produced using a wild-type genome.

We also surprisingly found that MAAP appears to affect degradation of viral capsid proteins. MAAP may have direct proteolytic activity. Alternatively, MAAP may interact with another protein, or with the viral or host cell cellular genome, to inhibit viral capsid protein degradation. Alternatively, MAAP may affect cellular proteolysis or proteolytic activity against AAV capsid proteins.

We demonstrated that eliminating MAAP, or truncating its C-terminal end produces a virus preparation that is both higher in yield and more resistant to degradation than virus made from a wild-type genome.

We also found that impairing the expression of MAAP increases the percentage of full virions. We thus provide a way to improve the quality of manufactured virus. This is critically important in manufacturing human gene therapy vectors.

We predict that inactivating wild-type MAAP expression produces a virus that is better able to transduce target cells.

Given our disclosure here, the artisan can readily make certain modifications. For example, we truncate translation of full-length MAAP by inserting point-mutations in the underlying DNA. The artisan could achieve the same ends by, for example, co-transfecting the producer cell with a plasmid coding for interfering RNA that interferes with MAAP expression. Alternatively, the artisan could treat the producer cell with a monoclonal antibody directed to MAAP. Such approaches achieve the same end as point-mutating the viral genome, albeit perhaps at greater expense. We thus intend the legal scope of our patent to be defined not by our various examples, but by our appended legal claims.

### Example 2

Adeno-associated virus (AAV) was originally discovered as a contaminant of Adenovirus production¹. AAV serotype 2 is considered as the reference model and encodes a ssDNA genome of 4679 bases packaged in an icosahedral capsid. The AAV2 genome is flanked by GC-rich DNA regions structured in hairpin, Inverted Terminal Repeats (ITRs). ITRs are recognized by the AAV large Rep proteins, allowing AAV genome replication, but also its integration in the host chromosomal DNA in a site specific manner. The smaller Rep proteins are necessary for the genome packaging. Capsids are composed of VP1, VP2 and VP3 proteins with a ratio of approximately 1:1:10 at the population level and with a total of 60 VP per capsid. The capsid assembly and the transport of the VPs to the nucleus is mediated by the Assembly Activating Protein (AAP), also encoded on the capsid gene but on a different reading frame than the VPs. The Membrane Associated Accessory Protein (MAAP) is encoded on the cap gene in the region coding also for the VP1/2 unique domain, and is associated with the cell² and nuclear membranes (Example 1). MAAP accelerates wt-AAV2 replication. However, truncated C-terminal MAAP variants enhanced AAV2 production in plasmid based transfection production using an Adeno helper plasmid. Based on the results of Example 1, we have retained two AAV2 MAAP variants displaying potential interest for recombinant (r)AAV production, the MAAP-S33-S39-S47 mutated to stop codons (also referred to as a triple stop mutant) and the MAAP-L100 mutated to stop codon. Both mutant do not affect the VP1/2 protein sequences. Similar MAAP mutants were made in the cap gene encoding recombinant AAV serotypes 1, 2, 5, 6, 8 and 9, again without altering the VP1/2 amino acid sequence. In 293T cell line, viral genome (vg) yields were improved for all rAAV serotypes apart from rAAV5, particularly for cap genes encoding the MAAP-IUS100 variant. In these experiments the most drastic production increase was observed for rAAV6. The alteration of MAAP protein resulted also in the modification of the secretion profile of some rAAVs, resulting in rAAV6 and 8 present in the cell culture medium to be essentially retained inside the cells. DNA and protein sequences of MAAP of several AAV serotypes³ showed that almost all AAV cap genes could be implemented with MAAP-S33-S39-S47 and MAAP-L/S-100 mutations, without affecting their VP1 protein sequences. Constructing a phylogenetic tree of the MAAP shows two main clades of AAV that could be associated with specific properties of AAV serotypes.

Material and Methods

Virus Preparations

rAAV were constructed as described in Example 1. rAAV vectors were prepared as follows. 293T cells (European Collection of Cell Cultures 293T Number: 12022001) were grown in Dulbecco's modified Eagle medium (DMEM, Gibco 11965084) supplemented with 10% fetal bovine serum (FBS, Thermo Fisher 10091-148), supplemented with 2 mM L-glutamine (Gibco, 25030-024), and penicillin-streptomycin (Gibco 15070-063). Polyethylenimine (PEI) transfections of rep-cap plasmid, mSeAP-ITR plasmid and adenovirus helper plasmid (1:1:1 ratio, total 261 ng/cm²) were performed on 293T cells in 6-well plates (341000 cells/well). Alternatively pDG2, pDP6, pDP5rs, and pDP8 plasmids from Plasmid Factory were used in combination with the mSeAP-ITR plasmid (1:1 ratio, total 261 ng/cm²). The PEI Pro (Polyplus Transfection, ref #115-100)/DNA weight ratio was maintained at 1:1 in serum-free DMEM medium. Serum free media exchange was carried 24 h post transfection. rAAV were harvested 72 h after transfection. For viral genome titer determination and rAAV capsid ELISA samples, rAAV were harvested using Triton-X-100 buffer (0.5% Triton-X-100 (Sigma-Aldrich, ref #X100-1L) and 2 mM MgCl2 (Merck, ref #E13980) in 1×PBS (Gibco, rem 18912-014)) and Denarase (50 U/ml, c-Lecta, rem 20804-5M). Lysis buffer was added to the media and cells were incubated for 2 h at 37° C. before cell lysate was collected.

Quantification of rAAV Genomes by Droplet Digital PCR

To obtain droplet digital PCR (ddPCR) AAV vg titers, crude preparations of virus were DNaseI (0.01 U/μl, Invitrogen, rem 18047-019) and Proteinase K (0.1 μg/μl, Roche, rem 03115879001) treated, and viral titers were obtained by ddPCR amplification (QX200, Bio-Rad) with primers (CMV-FWD [5′-CATGACCTTATGGGACTTTCCT]; CMV-REV [5′-CTATCCACGCCCATTGATGTA]) and probe (CMV-PRB [5′-6-FAM/TCGCACCTG/ZEN/ATTGCCCGACATTAT/IABkFQ) detecting the CMV promoter driving the mSeAP expression cassette. All primers and probes were ordered from Integrated DNA Technologies. For mastermix generation, primers (900 nM) and probe (250 nM) were diluted in 2×ddPCR Supermix for Probes (no dUTP, Bio-Rad, ref #1863025) and nuclease free water (Thermo Scientific, remR0582).

ELISA

To determine the ratios of capsids containing rAAV genomes from AAV capsids, capsid ELISA was first performed on 500-10,000 serial dilutions of the virus preparation using AAV titration ELISA kits according to the manufacturer's instructions. For rAAV2, Progen, PRAT; for rAAV5, Progen PRAAV5; for rAAV6, Progen PRAAV6; for rAAV8, Progen PRAAV8. Ratios of capsids containing the genome of interest versus the total capsids, expressed as percentages, were then calculated by dividing rAAV vg titers by capsid titers.

Statistical Analysis

The statistical analyses were performed using GraphPad software (Prism). The significance values shown above the bars on the figures are denoted as **** (p<0.0001), *** (p<0.001), ** (p<0.01), * (p<0.05) or ns (not significant).

Phylogenetic Analysis

Cap gene sequences originally used by Gao and collaborators³ with accession number AY530553 to AY530629 were annotated for the MAAP ORF. The CTG codon found in the MAAP ORF was used to as start codon used for the translation of MAAP protein as found in 2 and for wt-AAV2 (Example 1 of this patent application). Alignment and phylogenetic reconstructions were performed using the function “build” of ETE3 v3.1.1⁶ as implemented on the GenomeNet (https://www.genome.jp/tools/ete/). Alignment was performed with MAFFI v6.861b with the default options⁷ or Multalin⁴. The tree was constructed using FastTree v2.1.8 with default parameters⁸. Graphical representation was performed using iTOL⁹.

Results

MAAP Variants Increase rAAV Productivity.

We generated rAAV of serotypes 1, 2, 5, 6, 8 and 9 encoding the mSeAP gene. The cap gene either encoded wild-type (wt)-MAAP of the respective serotypes, or the MAAP-S33-S39-S47 mutated to stop for rAAV1, 2, 6, 8 and 9 (corresponding to MAAP-S59-65-71 for AAV5) in the cap gene. Those MAAP mutants are referred to as MAAP triple stop herein. Lastly, we produced rAAV2 and 9 using MAAP-L100 to stop variant and rAAV1, 6 and 8 using MAAP-S100 to stop variant. Those variants are often referred to MAAP-US-100 in the text. Mutations introduced in the cap gene to obtain the MAAP triple stop and MAAP-U/S-100 variants did not affect the amino acid sequence of VP1/2 proteins. So, for all the AAV serotypes studied in this work can be introduced the MAAP-triple stop mutation, i.e a stop codon in the MAAP sequence in place of codons coding for S33-S39-S47 (for AAV5 those mutants correspond to S59-65-71), without modifying the amino acid sequence of the VPs. Likewise, all AAV serotypes can have their MAAP-L/S-100 mutated to stop codons without modifying the VP amino acid sequence. For AAV5 the equivalent mutation is found in MAAP-S123. For AAV hu.28, it corresponds to MAAP-W100.

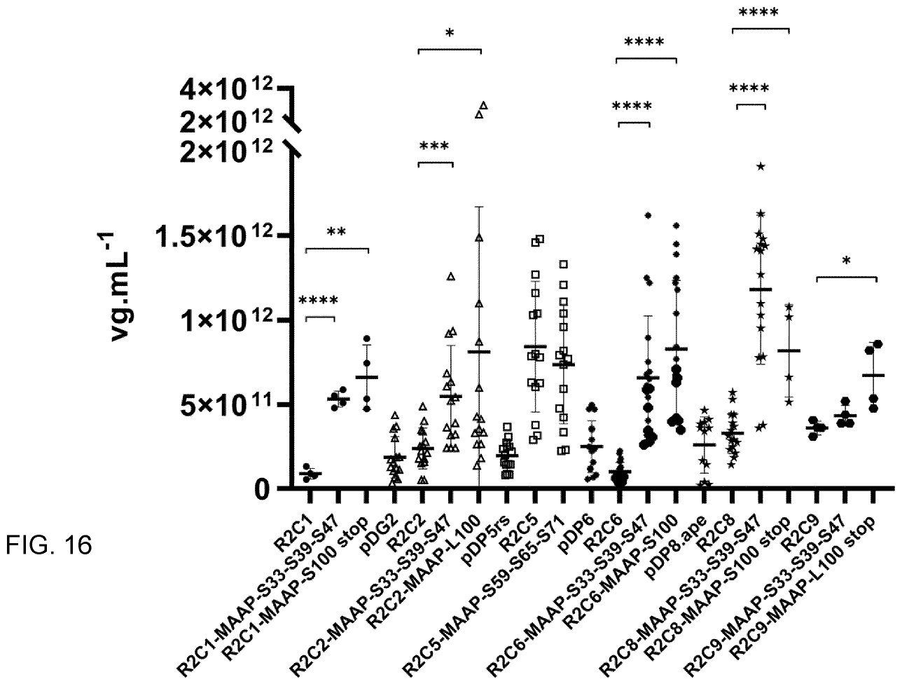

The triple stop rAAV1 encoding mSeAP increased viral genome titers (vg) 6.02-fold over wt-MAAP, while MAAP-S100 variants resulted in a 7.47-fold increase (FIG. 16 and table 18). For rAAV2-mSEAP, the MAAP triple stop increased viral genome titers (vg) 2.30-fold, while MAAP-L100 variant resulted in a 3.41 increase over the wt-MAAP (FIG. 16 and table 18) For rAAV6-mSeAP, the figures were 6.51 and 8.20-fold, respectively, and for AAV8-mSeAP, 3.60-fold and 2.49-fold increase was obtained. For AAV9, variants MAAP-S33-S39-847 and MAAP-L100 resulted in 1.21-fold and 1.87-fold increase in vg titers, respectively, over the wt-MAAP sequence. In the case of rAAV5-mSeAP, the same modifications did not affect the vg titers. In conclusion, we observed a global increase in the rAAV productivity for rAAV1, 2, 6, 8 and 9, but not 5, when MAAP triple stops and MAAP-L/S-100 variants were used over the wt-MAAP.

Impact of MAAP Variants on rAAV Genome Packaging

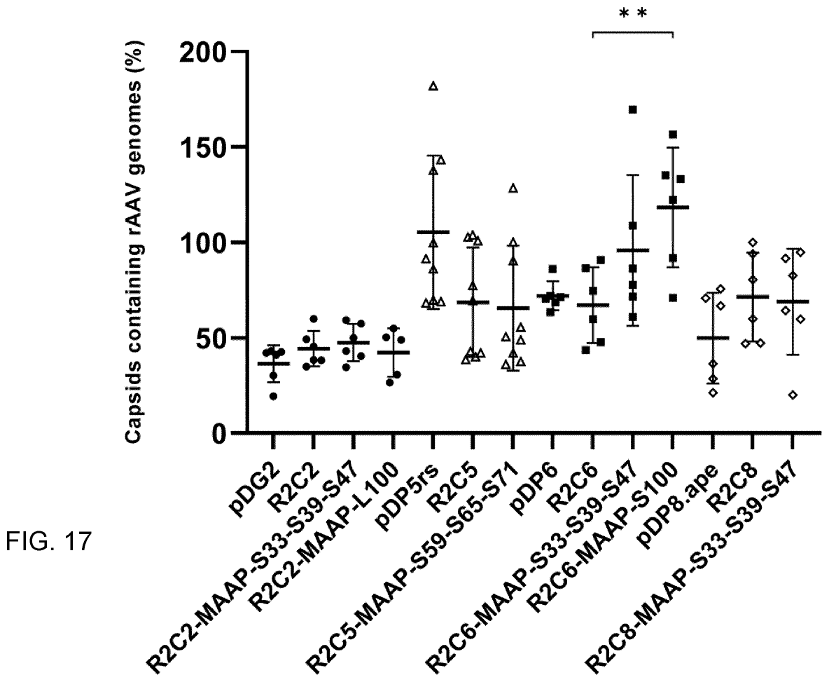

We studied also the role of the above described MAAP mutations on the percentage of capsids containing the transgene of interest versus the total capsids (full viruses) and found not a significant difference between the mutants and wt-MAAP encoding serotype 2, 5, 8 viruses (FIG. 17 and table 19). However, rAAV6 showed an increase in the level of full viruses when MAAP variants were used (FIG. 17 and table 19). When a cap6 gene encoding the wt-MAAP was used, we measured on average 67.3% of capsids containing the rAAV genome, and 95.9% and 118.4%, respectively, when the cap6 gene was encoding MAAP-S33-S39-S47 and MAAP-S100 variants.

MAAP Variants Affects Eggress of rAAV

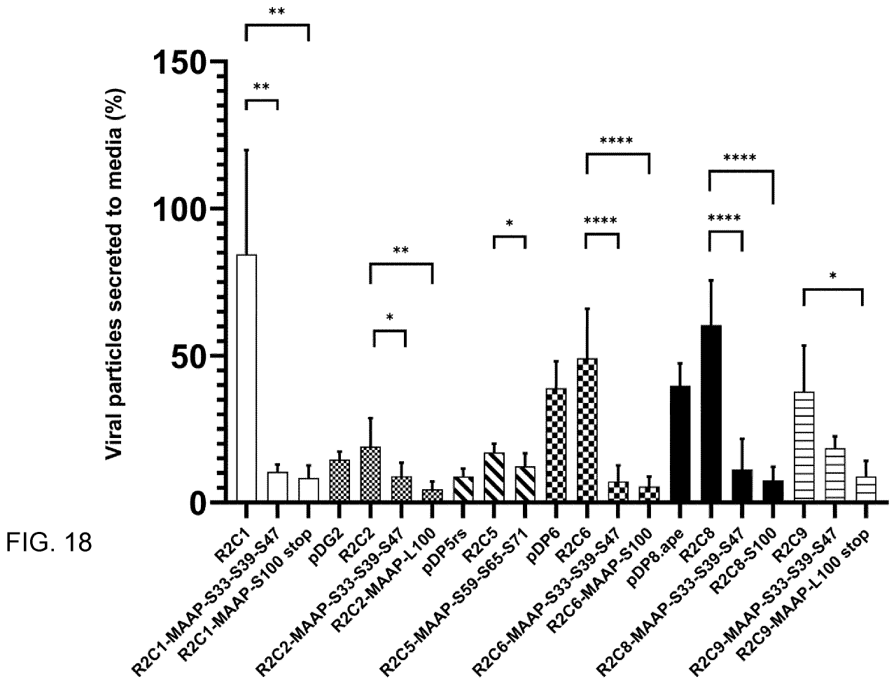

Next we studied how different rAAV serotypes egress into cell culture medium and observed that both MAAP-triple stops and MAAP-L/S100 variants led to a marked reduction of rAAV quantities in the cell media when compared to intracellular rAAV (FIG. 18 and table 20). For rAAV1 and 6, the proportion of vector found in the cell culture media prior to harvest was of 84.51% and 49.14% respectively. This proportion of vector found in the cell culture media felt to 10.45% for MAAP-triple stop and 8.31% for MAAP-S100 variants, respectively, for serotype 1, and 7.15% and 5.49% in the case of AAV6, respectively. Similar results were obtained for rAAV8, wt-MAAP resulting in 60.38% of rAAV8 in the cell culture media while the triple stop variant led to 11.33% and MAAP-S100 7.55% of the recombinant AAV present in the cell culture. A significant reduction in the proportion of modified rAAV2 and 5 in culture medium was also observed.

Phylogenetic Analysis of MAAP Across AAV Serotypes

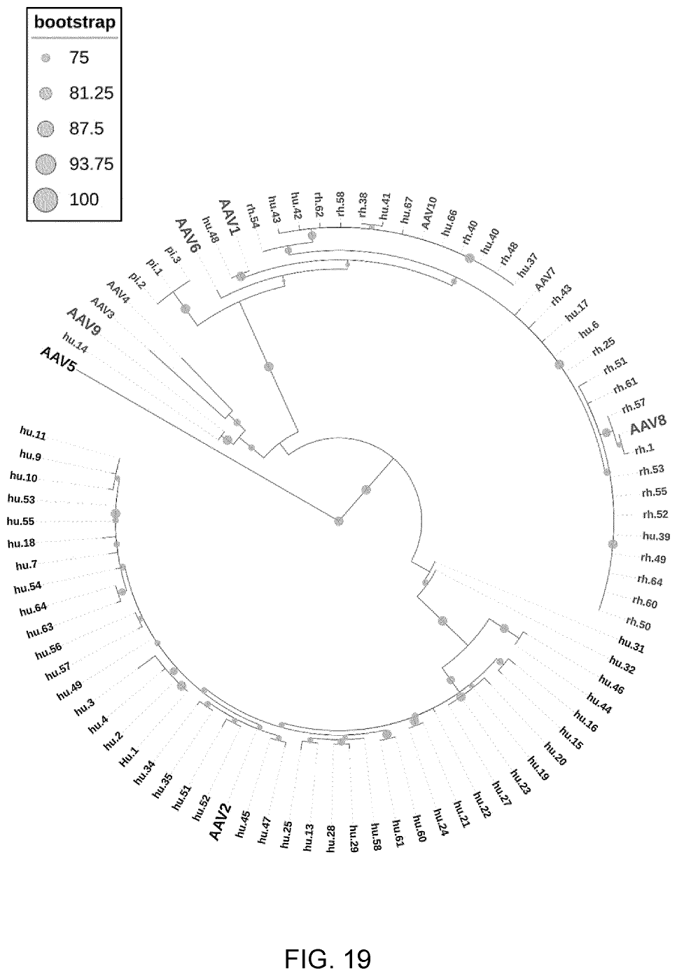

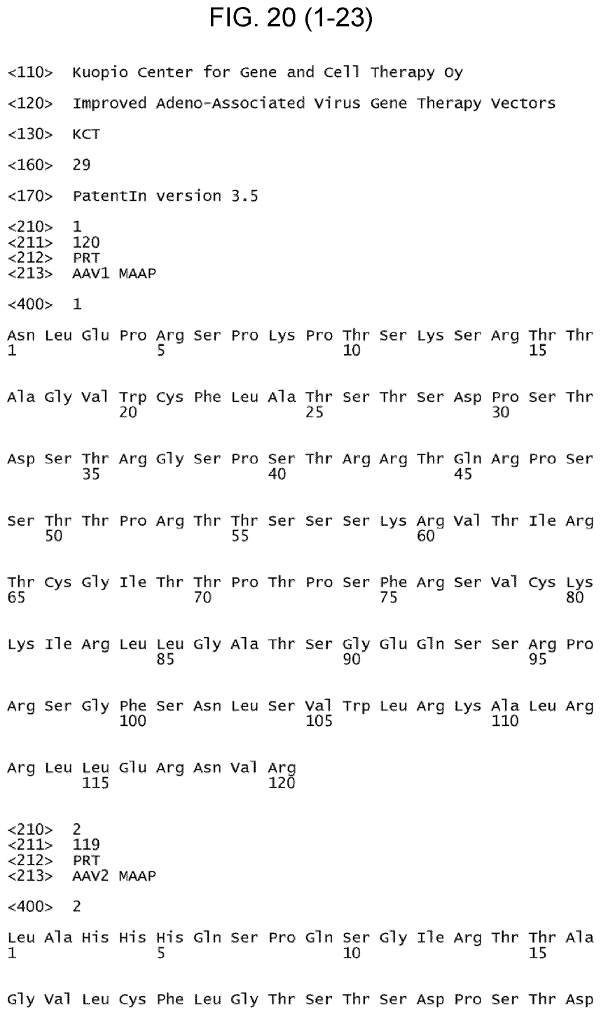

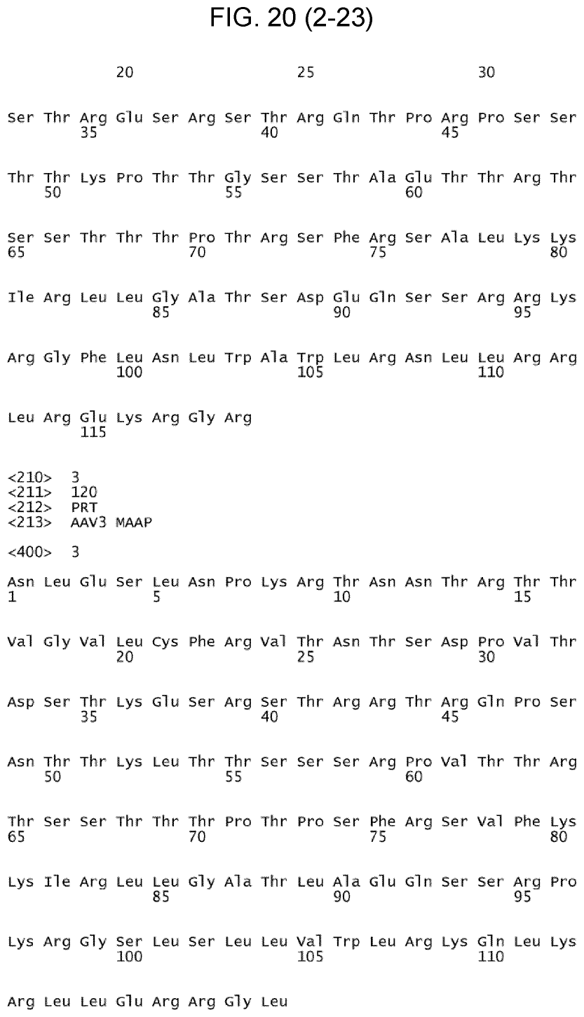

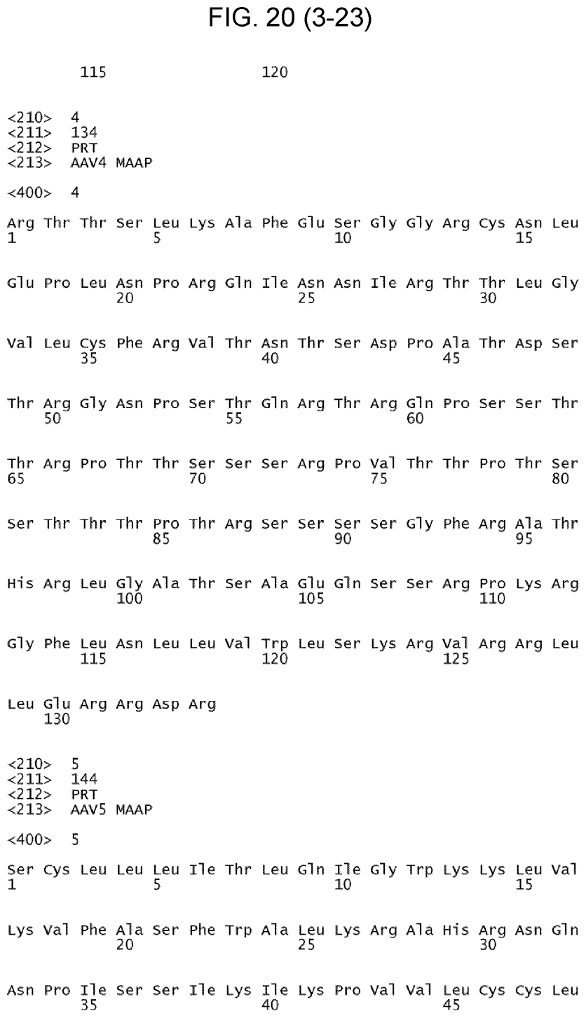

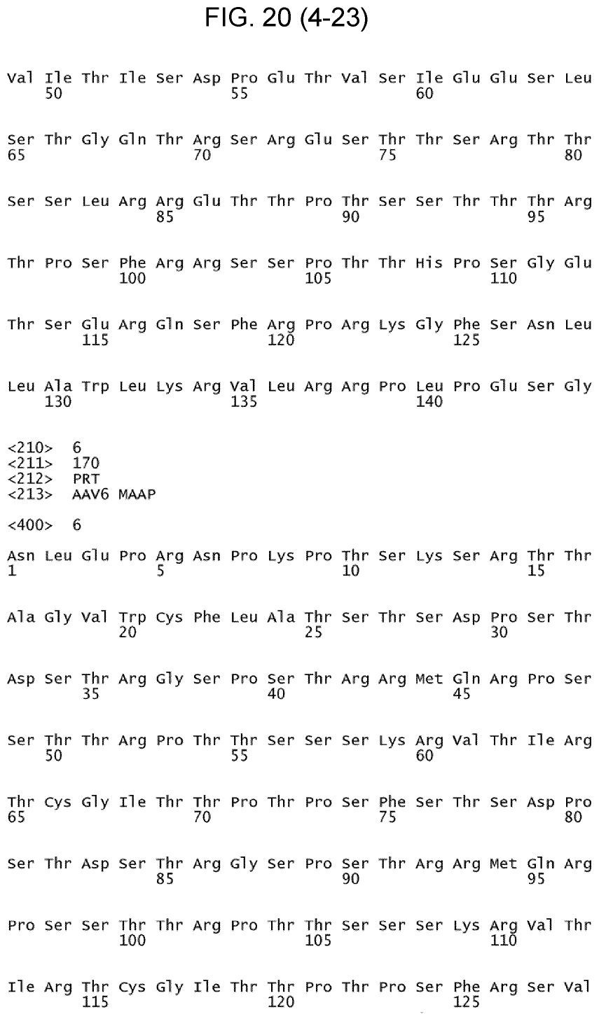

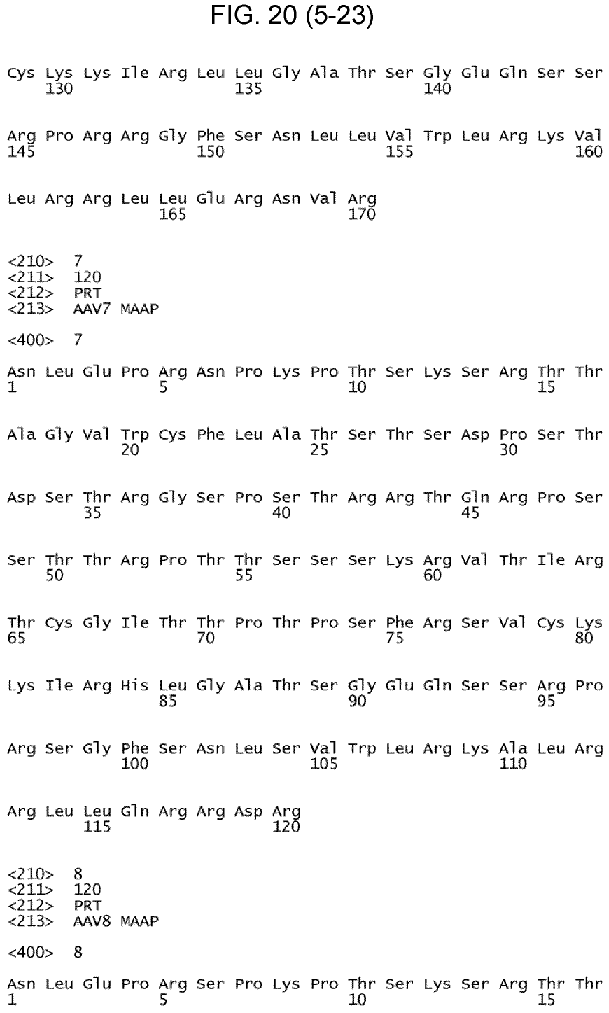

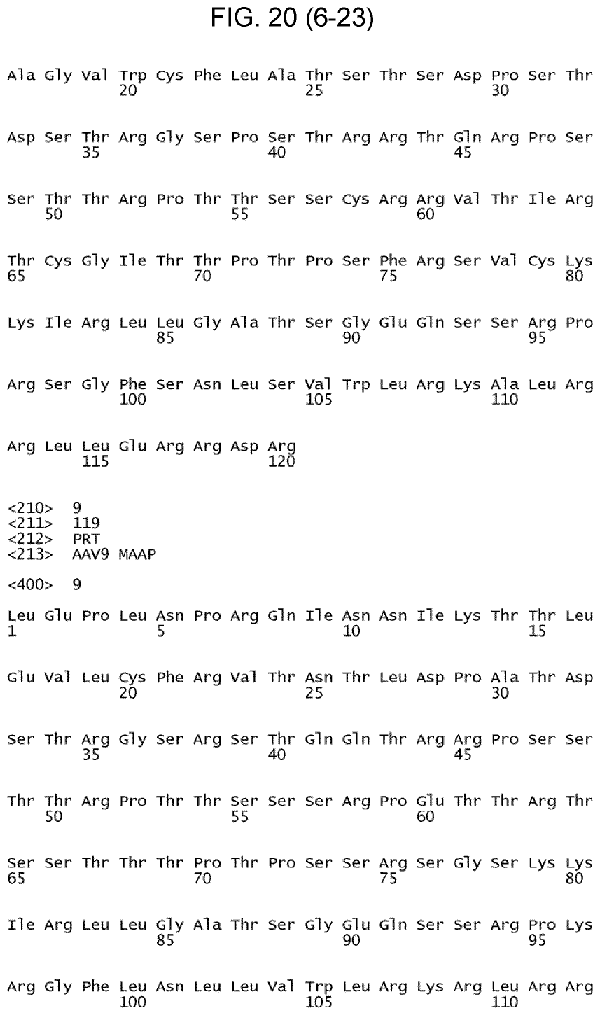

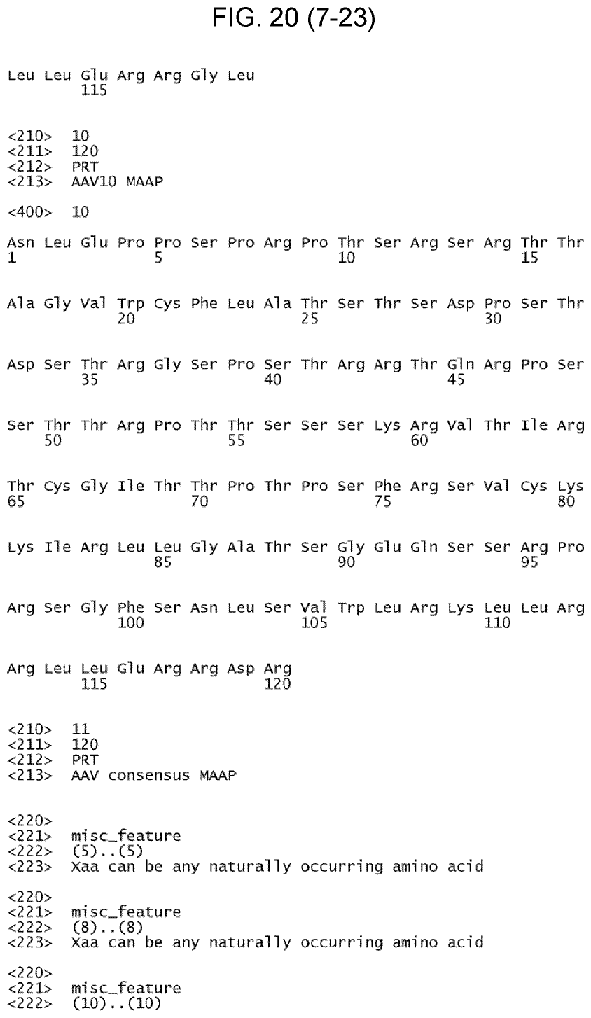

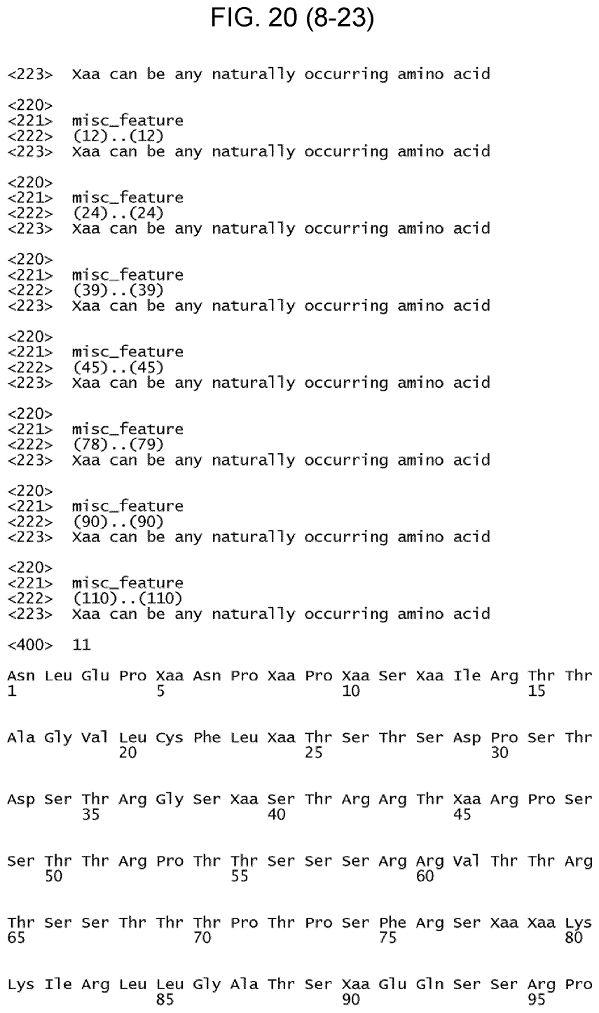

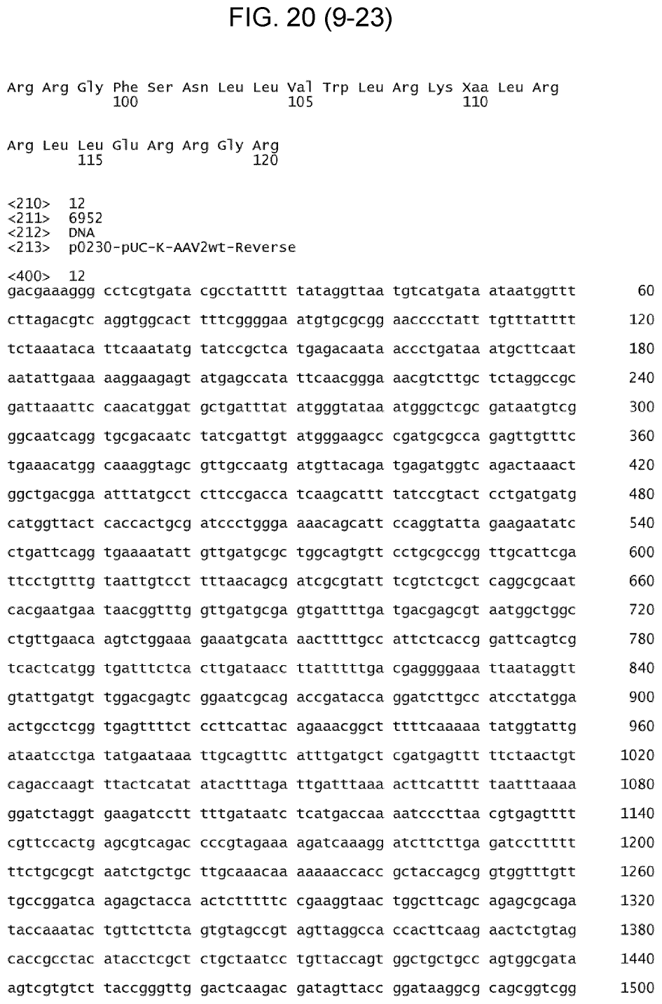

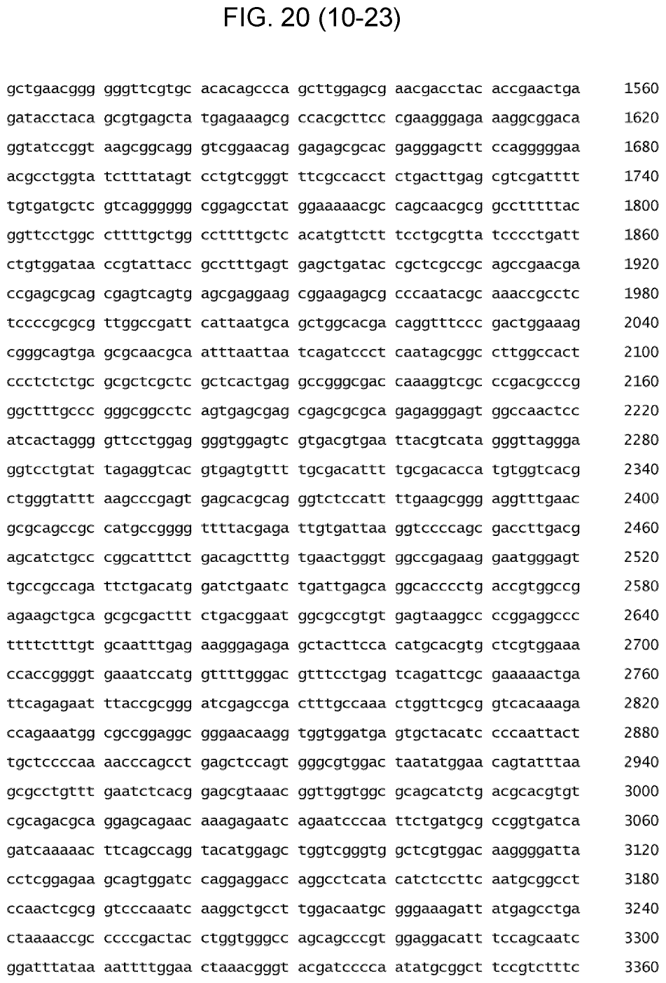

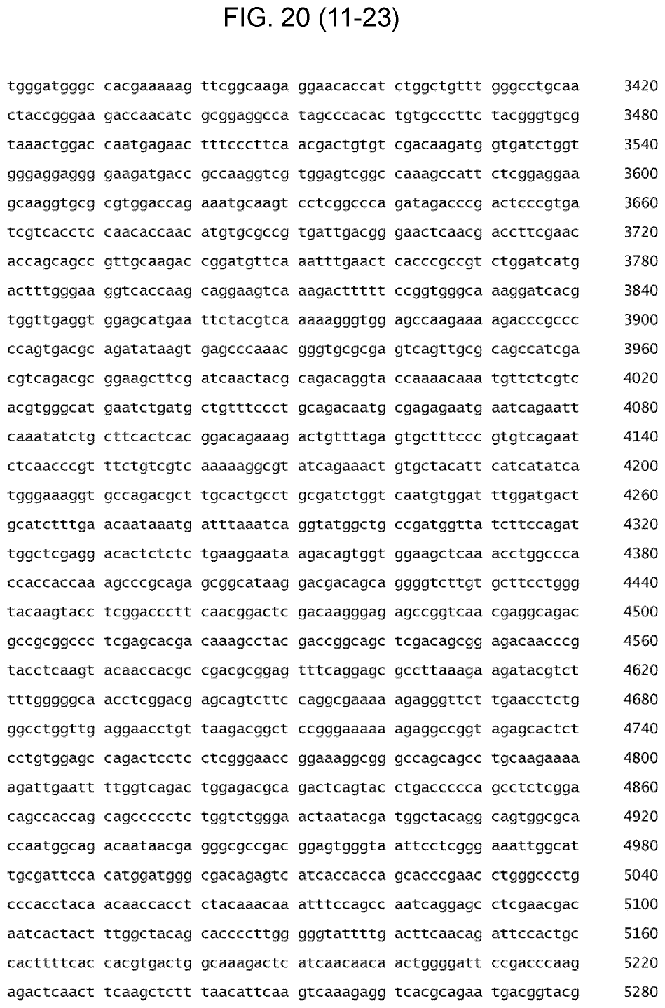

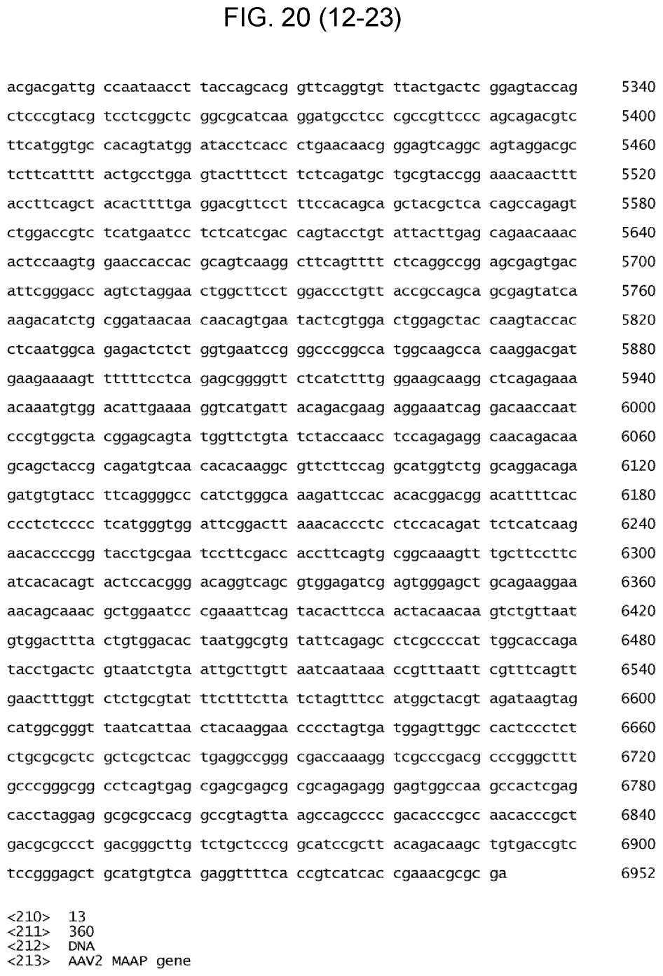

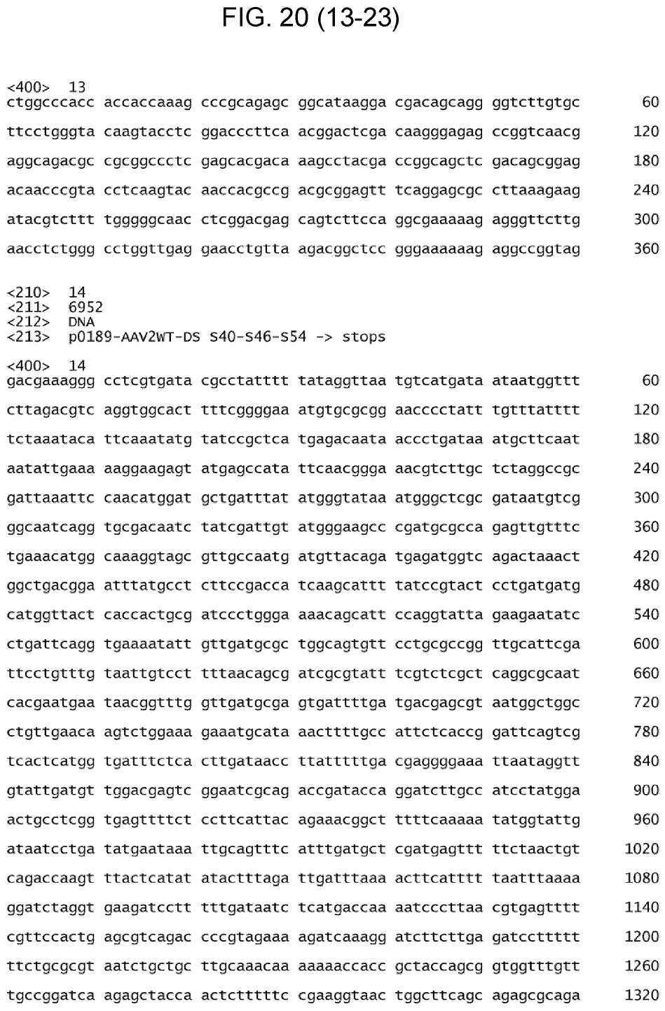

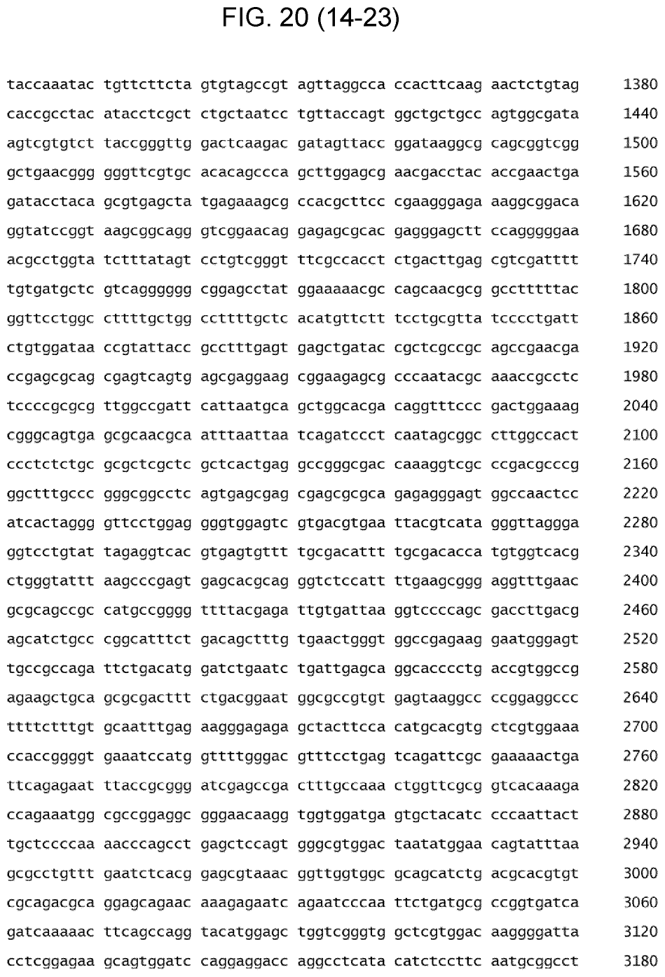

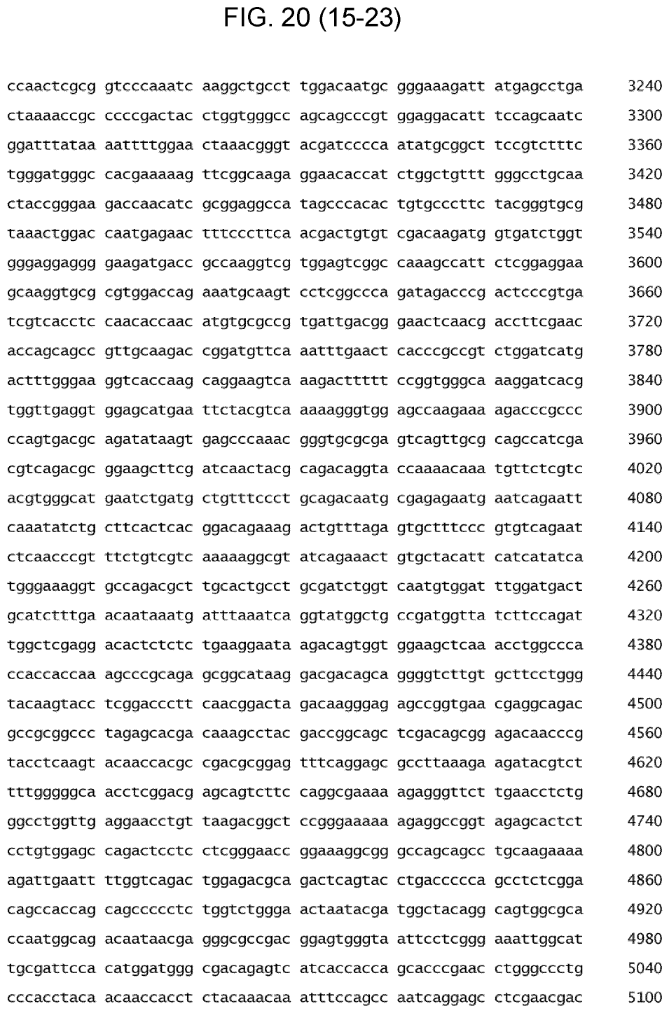

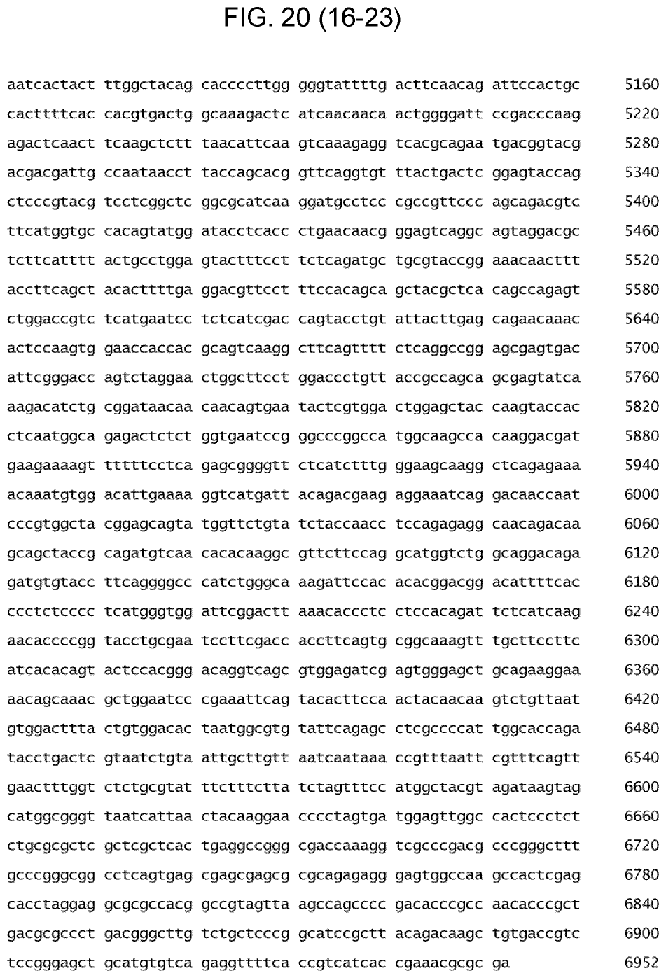

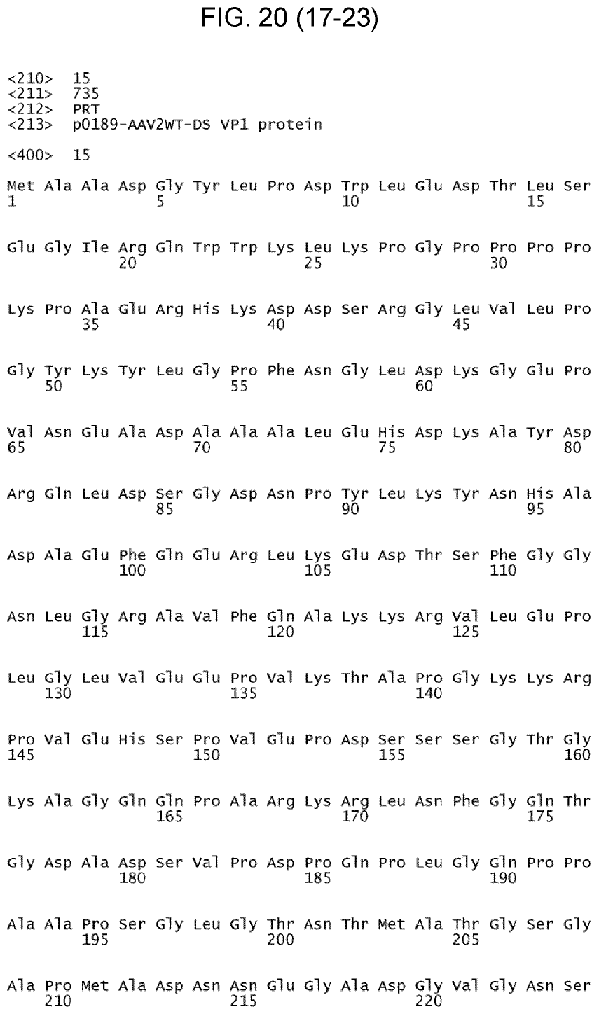

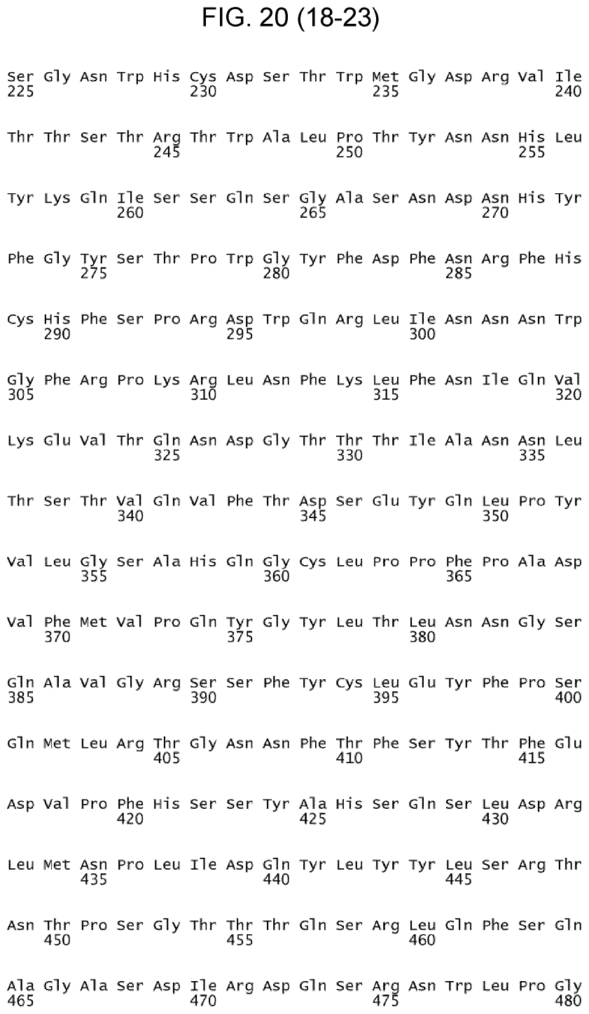

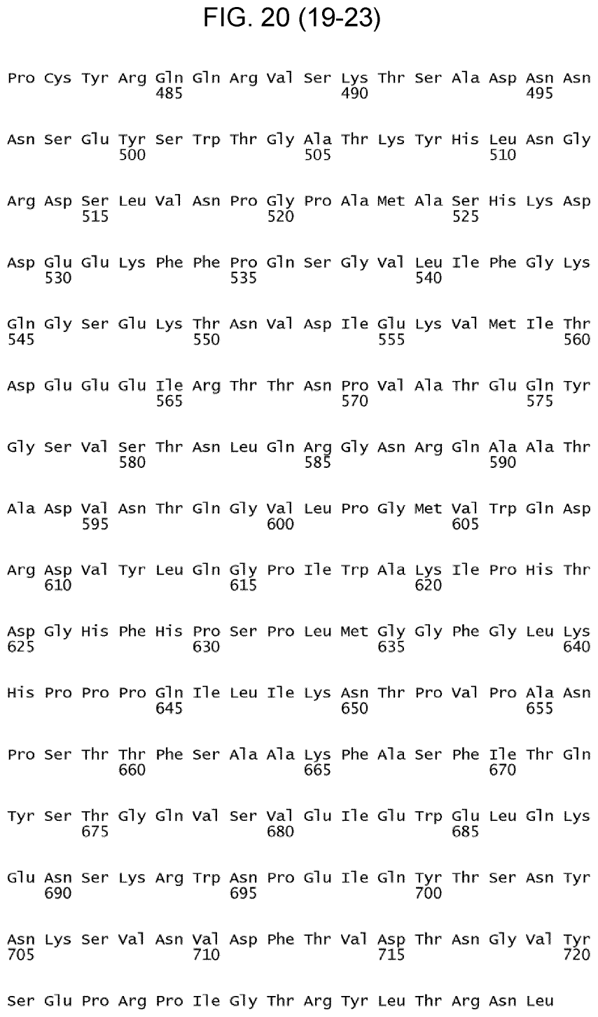

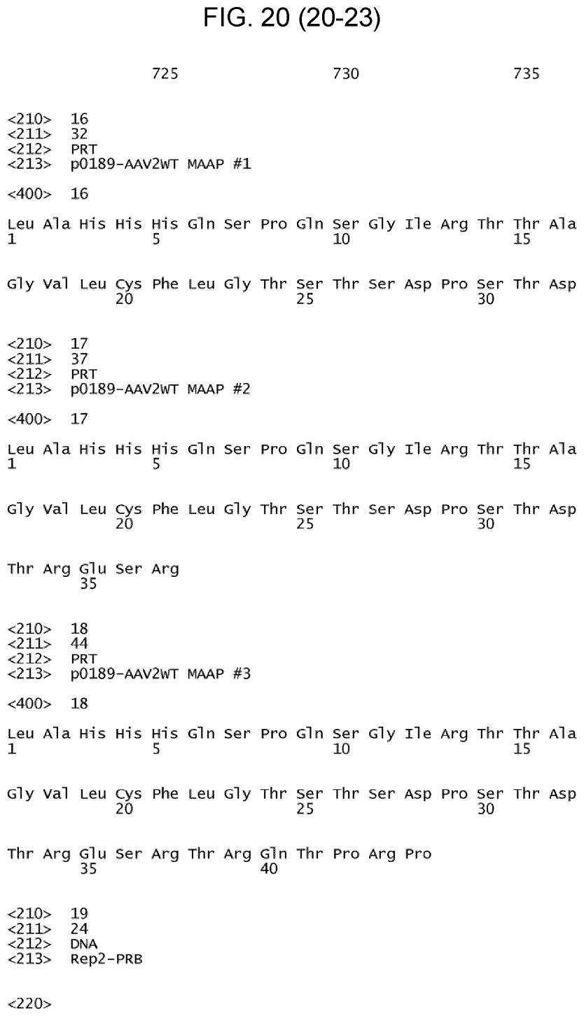

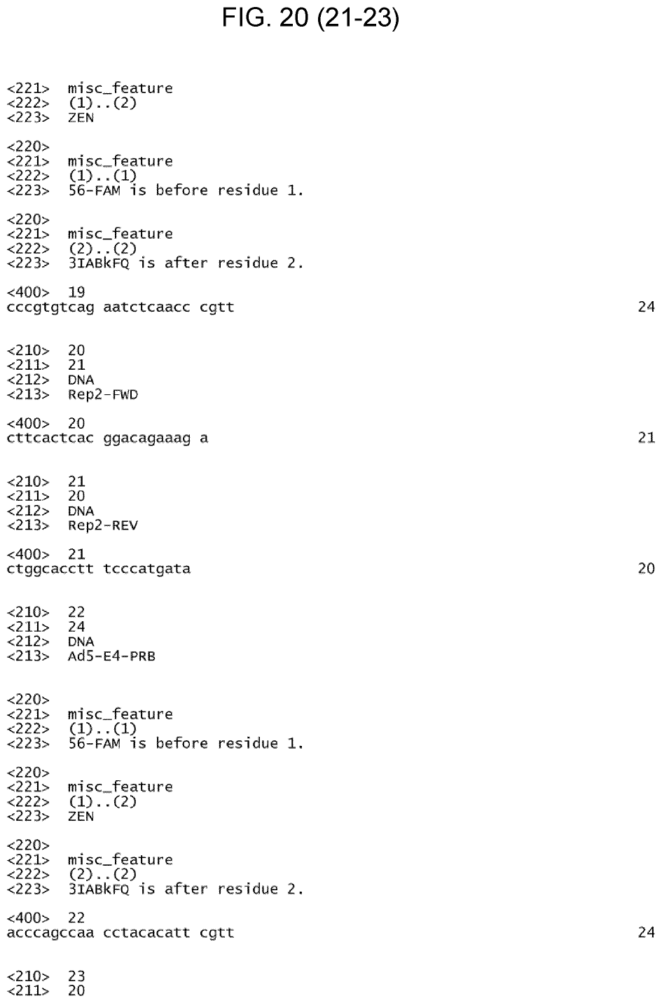

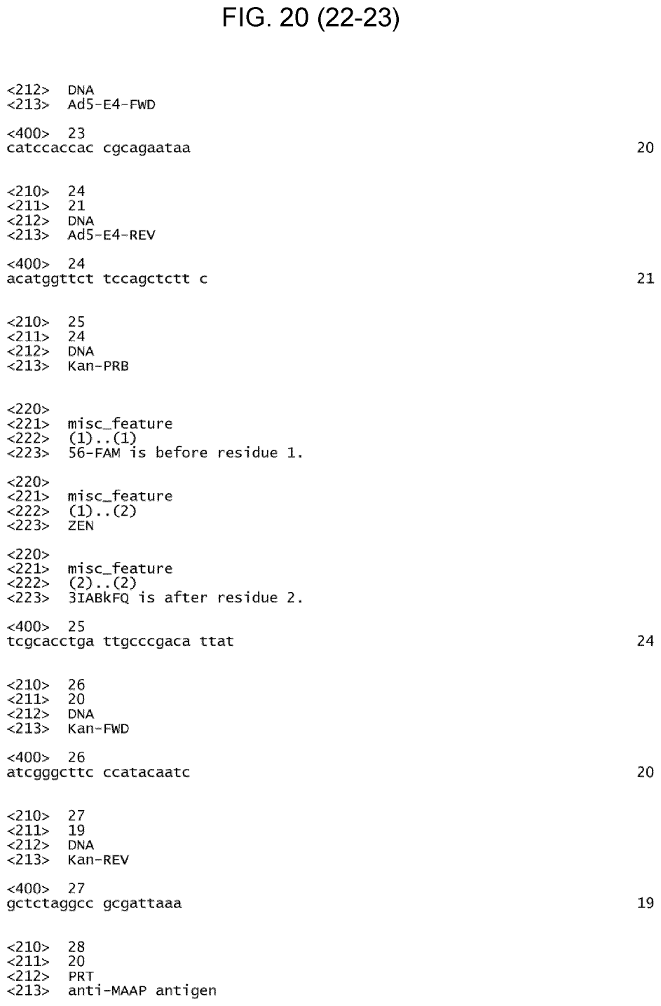

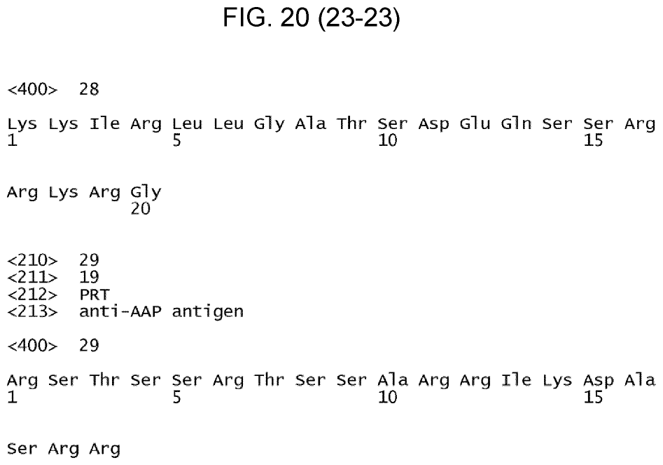

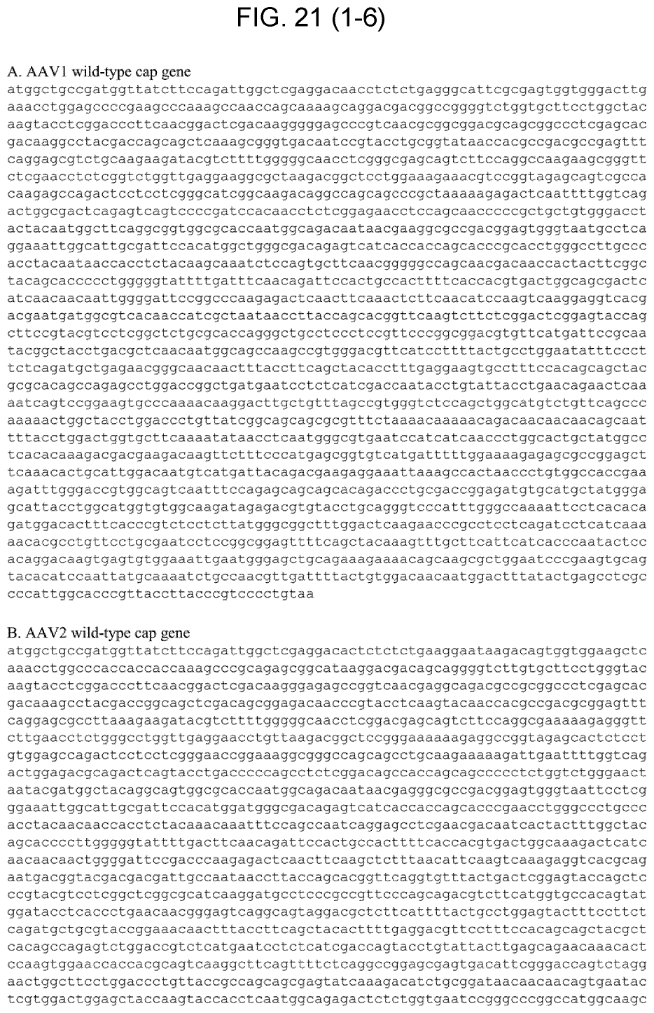

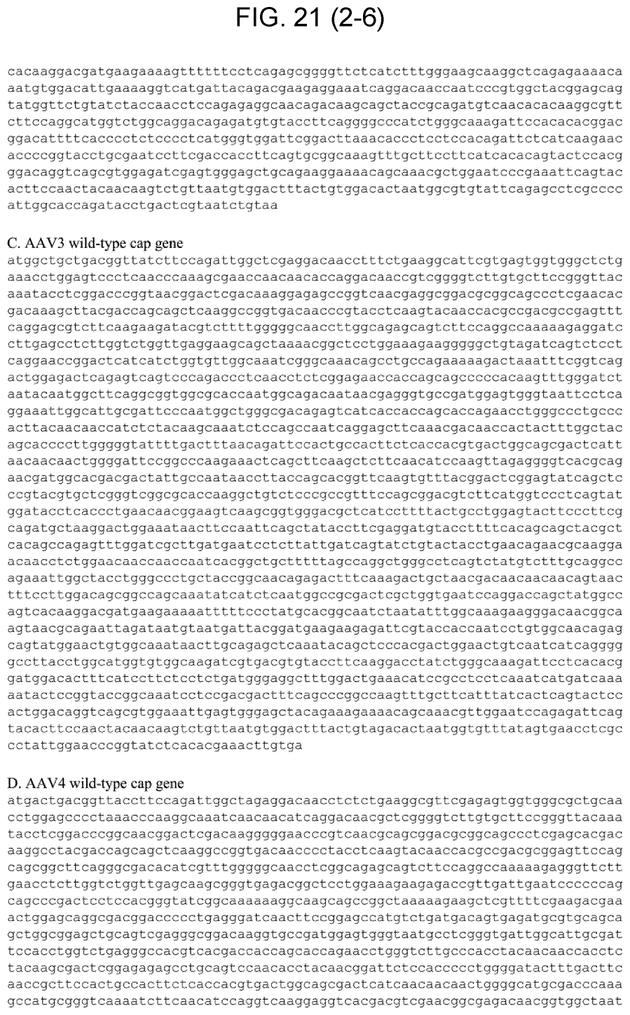

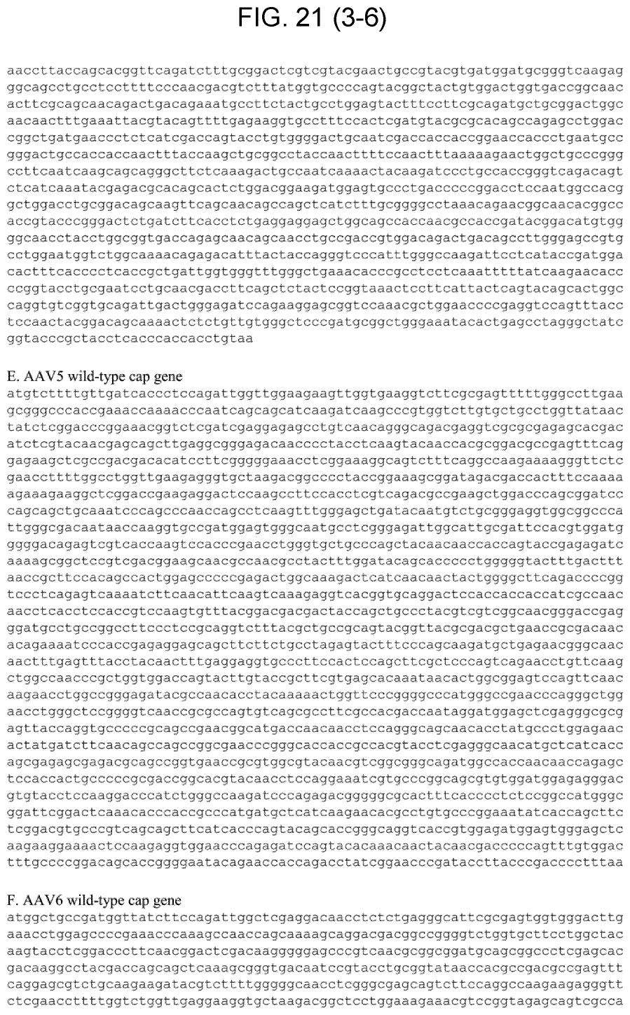

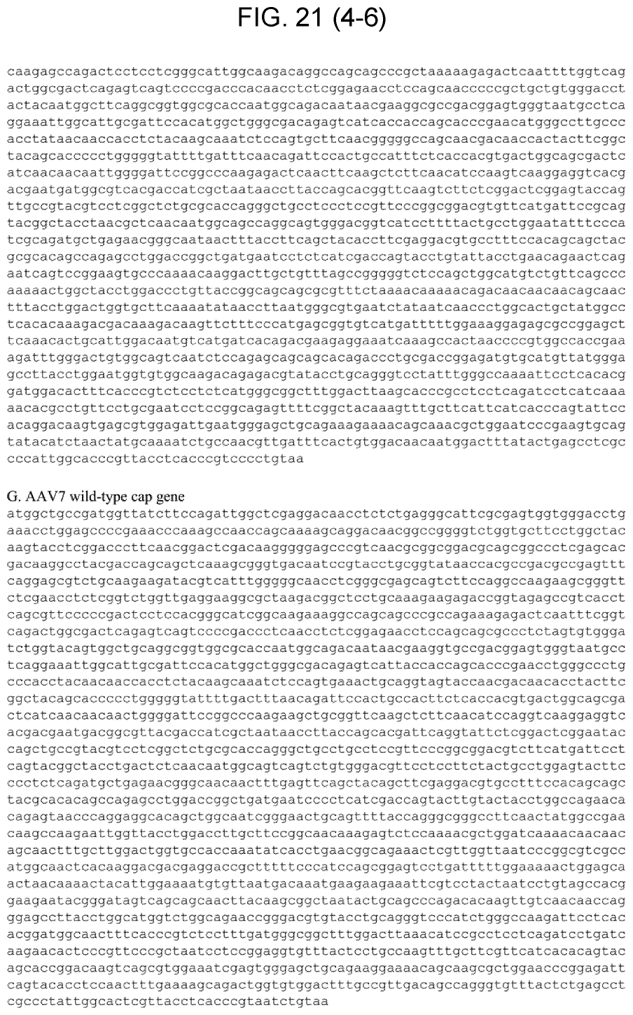

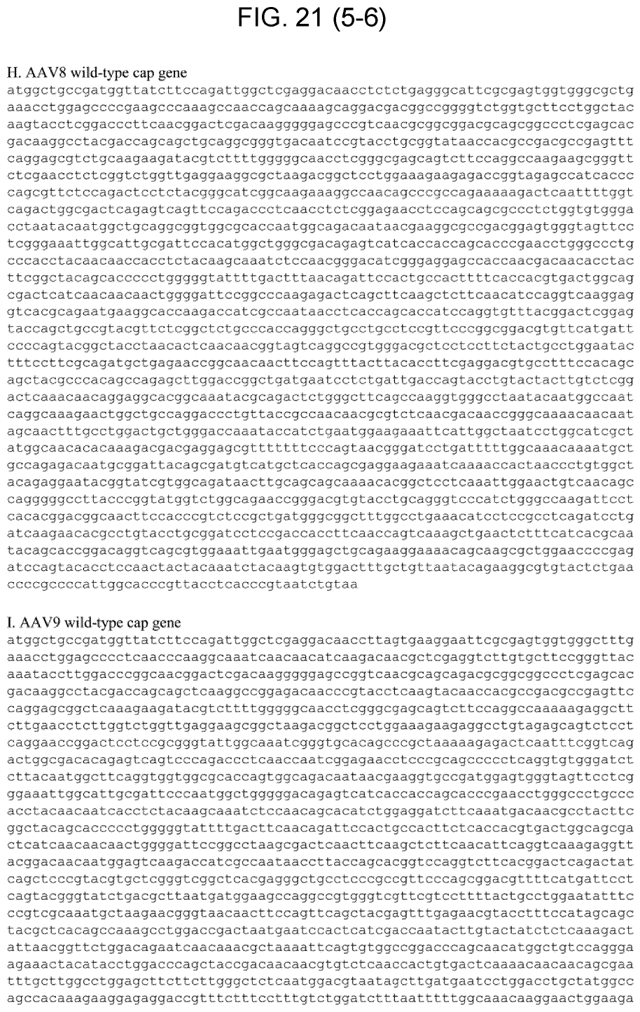

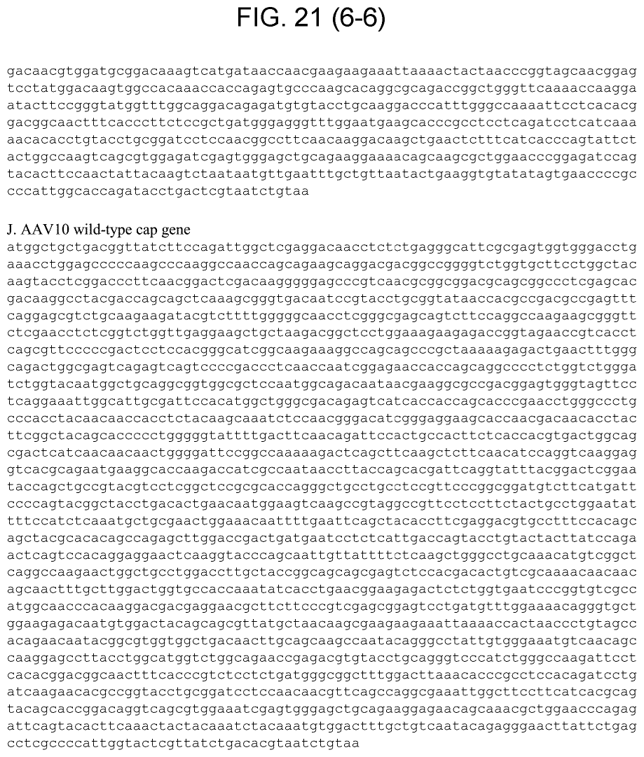

A MAAP phylogenetic tree was constructed of 87 AAV serotypes of human and non-human primate origins (FIG. 19). The MAAP sequences were obtained from the cap genes of the different clades of AAV previously defined based on the VP1 protein sequences 3.

A major fork divides AAVs in two different large groups plus a branch represented by AAV5. Clade A is represented by AAV1, 3, 4, 6, 8, 9, 10 and all non-human primates AAVs. Clade B is represented by AAV2 and AAV serotypes isolated from humans. AAV5 forms a separated branch by itself. Interestingly, AAV1, 6, 8 and 9, members of MAAP clade A, were characterised by at least 40% of the produced AAV found in the cell culture medium. In comparison, AAV2, a member of clade B, had less than 20% of the capsids containing the recombinant AAV genome found in the cell culture medium as was the case with AAV5, present on a major separated branch of the AAV phylogenetic MAAP tree. Thus, clade A AAVs seem to be more secreted compared to the member(s) of clade B and AAV5. However, further studies are needed to confirm this initial result by studing more members of clade B.

All studied members of MAAP clades A and B yielded higher vg titers when MAAP variants were used in rAAV production, AAV5 being the only exeption. We did not observed higher vg titer when MAAP-S59-S65-S71 variant was used. As AAV5 MAAP forms an isolated, own clade without other members compared to the many AAV members of clades A and B, it could be that MAAP-S59-S65-S71 mutant has different biological activities. However, because of having the most divergent MAAP, more extensive characterization of the right site of modification could still lead to productivity increase.

Interestingly, beside the AAV5 MAAP, all the other MAAP proteins align, without any gaps in the alignment, except AAV hu.57. It lacks one histidine at the beginning of the MAAP sequence (supporting file MAAP protein alignment). The results show a high level of MAAP conservation throughout the AAV family. The alignment highlight the main MAAP clades A and B, with members possessing highly related protein sequences. We also analysed whether the MAAP mutants could be transposed to other AAV serotypes, without modifying the original VP1 amino acid sequence. For all the studied serotypes presented in FIG. 19, we were able to perform the MAAP Triple-stop mutation without interfering with VP1 amino acid sequence, excluding the cap gene of AAV serotypes hu.15 and hu.16, belonging to MAAP clade B.

Table 18 shows the mean vg·mL⁻¹ titer of rAAV produced at 72 hpt, the fold difference in respect to rAAV made with wt-MAAP and MAAP variants within the same capsid serotype, and the repeats of each experiment (N) assessed in the study. Samples from left to right are. Samples from left to right are. rAAV1-mSeAP, produced 3-plasmid system, using Rep2-Cap1 plasmid encoding wt-MAAP; MAAP-S33-S39-S47; and MAAP-S100. rAAV2-mSeAP, produced using pDG2 (Plasmid Factory), 3-plasmid system, using Rep2-Cap2 plasmid encoding wt-MAAP; MAAP-S33-S39-S47; and MAAP-L100. rAAV6-mSeAP, produced using pDP6 (Plasmid Factory), 3-plasmid system, using Rep2-Cap6 plasmid encoding wt-MAAP; MAAP-S33-S39-S47; and MAAP-S100. rAAV5-mSeAP, produced using pDP5rs (Plasmid Factory), 3-plasmid system, using Rep2-Cap5 plasmid encoding wt-MAAP; and MAAP-S59-S65-S71. rAAV8-mSeAP, produced using pDP8.ape (Plasmid Factory), 3-plasmid system, using Rep2-Cap8 plasmid encoding wt-MAAP; MAAP-S33-S39-S47 and MAAP-S100. rAAV9-mSeAP produced with 3-plasmid system, using Rep2-Cap9 plasmid encoding wt-MAAP; MAAP-S33-S39-S47 and MAAP-L100.

TABLE 18

| rAAV vg titers | | R2C1-MAAP- | R2C1-MAAP- | | | R2C2-MAAP- | R2C2- |
|---|---|---|---|---|---|---|---|
| at 72 hpt | R2C1 | S33-S39-S47 | S100 stop | pDG2 | R2C2 | S33-S39-S47 | MAAP-L100 |
| mean (vg.mL⁻¹) | 8.85 × 10¹⁰ | 5.33 × 10¹¹ | 6.61 × 10¹¹ | 1.86 × 10¹¹ | 2.38 × 10¹¹ | 5.48 × 10¹¹ | 8.12 × 10¹¹ |
| Fold difference vs R2CX | — | 6.02 | 7.47 | 0.78 | — | 2.30 | 3.41 |
| N | 2 | 2 | 2 | 6 | 6 | 6 | 6 |
| rAAV vg titers | | | R2C5-MAAP | | | R2C6-MAAP- | R2C6- |
| at 72 hpt | pDP5rs | R2C5 | S59-S65-S71 | pDP6 | R2C6 | S33-S39-S47 | MAAP- S100 |
| mean (vg.mL⁻¹) | 1.95 × 10¹¹ | 8.43 × 10¹¹ | 7.37 × 10¹¹ | 2.49 × 10¹¹ | 1.01 × 10¹¹ | 6.58 × 10¹¹ | 8.29 × 10¹¹ |
| Fold difference vs R2CX | 0.23 | — | 0.87 | 2.46 | — | 6.51 | 8.20 |
| N | 6 | 6 | 6 | 6 | 8 | 8 | 8 |
| rAAV vg titers | | | R2C8-MAAP- | R2C8-MAAP- | | R2C9-MAAP- | R2C9-MAAP |
| at 72 hpt | pDP8.ape | R2C8 | S33-S39-S47 | S100 stop | R2C9 | S33-S39-S47 | L100 stop |
| mean (vg.mL⁻¹) | 2.59 × 10¹¹ | 3.28 × 10¹¹ | 1.18 × 10¹² | 8.18 × 10¹¹ | 3.59 × 10¹¹ | 4.33 × 10¹¹ | 6.73 × 10¹¹ |
| Fold difference vs R2CX | 0.79 | — | 3.60 | 2.49 | — | 1.21 | 1.87 |
| N | 5 | 7 | 7 | 2 | 2 | 2 | 2 |

Table 19 shows the mean of capsids containing rAAV genome measured for each virus produced at 72 hpt, the fold difference in respect to rAAV of same capsid serotype but encoding wt-MAAP or MAAP variants, and the amount of technical replicates (N) assessed in the study. Samples from left to right are. rAAV2-mSeAP, produced using pDG2 (Plasmid Factory), 3-plasmid system, using Rep2-Cap2 plasmid encoding wt-MAAP; MAAP-S33-S39-S47; and MAAP-L100. rAAV6-mSeAP, produced using pDP6 (Plasmid Factory), 3-plasmid system, using Rep2-Cap6 plasmid encoding wt-MAAP; MAAP-S33-S39-S47; and MAAP-S100. rAAV5-mSeAP, produced using pDP5rs (Plasmid Factory), 3-plasmid system, using Rep2-Cap5 plasmid encoding wt-MAAP; and MAAP-S59-S65-S71. rAAV8-mSeAP, produced using pDP8.ape (Plasmid Factory), 3-plasmid system, using Rep2-Cap8 plasmid encoding wt-MAAP; MAAP-S33-S39-S47.

TABLE 19

| Capsids containing rAAV | | | R2C2-MAAP- | R2C2-MAAP- | | | R2C5-MAAP |
|---|---|---|---|---|---|---|---|
| genomes at 72 hpt (%) | pDG2 | R2C2 | S33-S39-S47 | L100 | pDP5rs | R2C5 | S59-S65-S71 |
| Mean % | 36.60 | 44.48 | 47.62 | 42.46 | 105.38 | 68.79 | 65.68 |
| Fold difference vs R2CX | 0.82 | — | 1.07 | 0.95 | 1.53 | — | 0.95 |
| N | 2 | 2 | 2 | 2 | 3 | 3 | 3 |
| Capsids containing rAAV | | | R2C6-MAAP- | R2C6- | | | R2C8-MAAP- |
| genomes at 72 hpt (%) | pDP6 | R2C6 | S33-S39-S47 | MAAP-S100 | pDP8.ape | R2C8 | S33-S39-S47 |
| Mean % | 72.11 | 67.30 | 95.92 | 118.37 | 50.04 | 71.54 | 69.00 |
| Fold difference vs R2CX | 1.07 | — | 1.43 | 1.76 | 0.70 | — | 0.96 |
| N | 2 | 2 | 2 | 2 | 2 | 2 | 2 |

Table 20 shows the mean percentage of secreted rAAV particles measured for each virus produced at 72 hpt, the fold difference in respect to rAAV of same serotype but produced with wt-MAAP or the MAAP variants, and the amount of technical replicates (N) assessed in the study. Samples from left to right are: rAAV1-mSeAP, produced with 3-plasmid system, using Rep2-Cap1 plasmid encoding wt-MAAP; MAAP-S33-S39-S47; and MAAP-S100. rAAV2-mSeAP, produced using pDG2 (Plasmid Factory), 3-plasmid system, using Rep2-Cap2 plasmid encoding wt-MAAP; MAAP-S33-S39-S47; and MAAP-L100. rAAV5-mSeAP, produced using pDP5rs (Plasmid Factory), 3-plasmid system, using Rep2-Cap5 plasmid encoding wt-MAAP; and MAAP-S59-S65-S71. rAAV6-mSeAP, produced using pDP6 (Plasmid Factory), 3-plasmid system, using Rep2-Cap6 plasmid encoding wt-MAAP; MAAP-S33-S39-S47; and MAAP-S100. rAAV8-mSeAP, produced using pDP8.ape (Plasmid Factory), 3-plasmid system, using Rep2-Cap8 plasmid encoding wt-MAAP; MAAP-S33-S39-S47 and MAAP-S100. rAAV9-mSeAP produced with 3-plasmid system, using Rep2-Cap9 plasmid encoding wt-MAAP; MAAP-S33-S39-S47 and MAAP-L100.

TABLE 20

| Viral particles secreted | | R2C1-MAAP- | R2C1-MAAP- | | | R2C2-MAAP- | R2C2-MAAP |
|---|---|---|---|---|---|---|---|
| to media at 72 hpt (%) | R2C1 | S33-S39-S47 | S100 stop | PDG2 | R2C2 | S33-S39-S47 | L100 |
| Mean % | 84.51 | 10.45 | 8.39 | 14.56 | 19.08 | 9.00 | 4.51 |
| Fold difference vs R2CX | — | 0.12 | 0.10 | 0.76 | — | 0.47 | 0.24 |
| N | 2 | 2 | 2 | 3 | 3 | 3 | 3 |
| Viral particles secreted | | | R2C5-MAAP- | | | R2C6-MAAP- | R2C6-MAAP |
| to media at 72 hpt (%) | pDP5rs | R2C5 | S59-S65-S71 | pDP6 | R2C6 | S33-S39-S47 | S100 |
| Mean % | 8.88 | 17.13 | 12.33 | 38.97 | 49.14 | 7.15 | 5.49 |
| Fold difference vs R2CX | 0.52 | — | 0.72 | 0.79 | — | 0.15 | 0.11 |
| N | 3 | 3 | 3 | 3 | 5 | 5 | 5 |
| Viral particles secreted to | | | R2C8-MAAP- | R2C8-MAAP- | | R2C9-MAAP- | R2C9-MAAP- |
| media at 72 hpt (%) | pDP8.ape | R2C8 | S33-S39-S47 | S100 stop | R2C9 | S33-S39-S47 | L100 stop |
| Mean % | 39.81 | 60.38 | 11.33 | 7.55 | 37.79 | 18.42 | 8.93 |
| Fold difference vs R2CX | 0.66 | — | 0.19 | 0.13 | — | 0.49 | 0.24 |
| N | 3 | 5 | 5 | 2 | 2 | 2 | 2 |

Conclusions

Gene therapy requires typically large vector doses. We present in this work the use of two MAAP variants that improve the rAAV productivity of AAV. The quality, based on the higher level of capsids containing the rAAV genome, was improved for at least rAAV6, which could improve the manufacturing capabilities of rAAV vectors. One major interest in the use of MAAP variants to improve rAAV productivity and quality is that those mutations can be implemented in the cap gene without modifying the VP amino acid sequence and the rAAV capsid properties.

Based on Example 1, we observed that two MAAP variants presented particularly desirable properties for the production of the virus. Introducing early stop codons in the MAAP ORF, at position MAAP-L100 or MAAP-S33-S39-S47, without changing the VPs amino acid sequence led to reduced capsid degradation, improved AAV2 productivity, and improved ratio of capsids containing the wt-AAV2 genome in comparison to total capsids. Although our focus was on those two particular mutants, several others led to similar improvement of productivity and quality of AAV2 and could be implemented for rAAV vectors production for almost all AAV serotypes, again without modifying the VP1 amino acid sequence, if desired.

This example focused on rAAV1, 2, 5, 6, 8 and 9, and for all of them, beside the serotype 5, the MAAP mutants improved the productivity of the serotypes. When AAV serotypes are classified based on MAAP phylogeny, AAV5 is isolated as the unique representative of his own branch. AAV 1, 6, 8 and 9 are grouped in clade A with the non-human primate AAVs and clade B groups includes AAV2 and other serotypes identified from human samples. Thus, AAV serotypes for which the MAAP is classified as a member of clade A and B would demonstrate higher titer productivity levels when MAAP variants are introduced in the cap gene. Besides allowing the maximum level of productivity to reach a range of 10¹² vg·mL⁻¹ when MAAP variants are used to produce rAAVs, another interesting property with the use of MAAP variants is the modification of the distribution profile of the rAAVs in the cell culture media or within the cells. For all the MAAP variants used to produce the different rAAV serotypes, we observed that the rAAV remained almost exclusively within the cells. This property of MAAP variants can be applied to AAV manufacturing and purification. Indeed AAV can be harvested only from the cells, if desired, and not from a combination of cells plus cell culture media. Processing only the cells for the purification of rAAV is associated with reduced manufacturing (purification) costs as lower volumes are processed. This saves costs in the use of regents such as DNase, allows cell lysis for vector harvest in defined buffers compared to cell culture media and uses lower liquid solution volumes. Lastly, the use of MAAP variants improves the levels of rAAV capsids containing the genome of interest, as seen particularly for rAAV6. This could lead to improve rAAV safety profiles by reducing the levels of empty capsids and capsids not containing the recombinant genome of interest. This could also reduce some purification steps in downstream processes as additional separation steps of the capsids containing the genome of interest with other capsids could become unnecessary.

Based on the DNA sequence of the cap gene in the region encoding the MAAP ORF, out of the 87 AAV serotypes we studied, all could produce the MAAP-triple stops and MAAP-L/S-100 mutations, without modifying the VP protein sequence. Thus, it is believed that the examples herein are applicable to all AAV serotypes.

### REFERENCES EXAMPLE 2

1. Atchison, R. W., Casto, B. C. & Hammon, W. M. D. Adenovirus-Associated Virus.

Science (80-.). 149, 754-755 (1965).
2. Ogden, P. J., Kelsic, E. D., Sinai, S. & Church, G. M. Comprehensive AAV capsid fitness landscape reveals a viral gene and enables machine-guided design. Science (80). 366, 1139-1143 (2019).
3. Gao, G. et al. Clades of Adeno-Associated Viruses Are Widely Disseminated in Human Tissues. J. Virol. 78, 6381-6388 (2004).
4. Corpet, F. Multiple sequence alignment with hierarchical clustering. Nucleic Acids Res. 16, 10881-90 (1988).
5. Girod, A. et al. The VP1 capsid protein of adeno-associated virus type 2 is carrying a phospholipase A2 domain required for virus infectivity. J. Gen. Virol. 83, 973-978(2002).
6. Huerta-Cepas, J., Serra, F. & Bork, P. ETE 3: Reconstruction, Analysis, and Visualization of Phylogenomic Data. Mol. Biol. Evol. (2016). doi:10.1093/molbev/msw046
7. Katoh, K. & Standley, D. M. MAFFT multiple sequence alignment software version 7: Improvements in performance and usability. Mol. Biol. Evol. (2013). doi:10.1093/molbev/mst010
8. Price, M. N., Dehal, P. S. & Arkin, A. P. Fasttree: Computing large minimum evolution trees with profiles instead of a distance matrix. Mol. Biol. Evol. (2009). doi:10.1093/molbev/msp077
9. Letunic, I. & Bork, P. Interactive Tree Of Life (iTOL) v5: an online tool for phylogenetic tree display and annotation. Nucleic Acids Res. (2021). doi:10.1093/nar/gkab301

1.-82. (canceled)

83. An adeno-associated virus genome that has a mutation that reduces expression of full-length wild-type membrane associated accessory protein (MAAP) yet maintains expression of VP1, wherein said mutation introduces at least one stop codon to stop translation of full-length wild-type MAAP.

84. The adeno-associated virus genome of claim 83, wherein the genome further has a mutation that inactivates the MAAP mRNA translation-initiation codon.

85. The adeno-associated virus genome of claim 83, wherein the mutation that reduces expression of full-length wild-type membrane associated accessory protein (MAAP) introduces at least one stop codon to stop translation of MAAP polypeptide at a polypeptide residue aligning with MAAP polypeptide consensus sequence SEQ ID NO. 11 from residue numbers 9 to 110, preferably from residues numbers 39 to 103.

86. The adeno-associated virus genome of claim 83, wherein the mutation that reduces expression of full-length wild-type membrane associated accessory protein (MAAP) introduces at least one stop codon to stop translation of MAAP polypeptide at a polypeptide residue aligning with MAAP polypeptide consensus sequence SEQ ID NO. 11
a) residue number 9, 33, 39, 47, 65, 90, 100, 103, 105, 106 or 110;
b) residue numbers 33, 39 and 47; or
c) residue number 100.

87. The adeno-associated virus genome of claim 83, where the genome is a naturally-occurring serotype, preferably, where the genome is selected from a serotype 1 genome, serotype 2 genome, serotype 5 genome, serotype 6 genome, serotype 8 genome and a serotype 9 genome.

88. The adeno-associated virus genome of claim 83, wherein the VP1 peptide sequence (a) is unaltered from wild type, or (b) contains a mutation.

89. The adeno-associated virus genome of claim 83, wherein the MAAP and VP1 peptide sequences each have at least 80% homology to wild type.

90. A producer cell that produces adeno-associated virus, the producer cell comprising the adeno-associated virus genome of claim 83.

91. The producer cell of claim 90, wherein the producer cell is eukaryotic, preferably selected from:
a) mammalian, preferably human cells;
b) yeast cells; or
c) insect cells.

92. A method for producing adeno-associated virus, the method comprising: obtaining an adeno-associated virus genome, and then introducing said genome into a cell to create the producer cell of claim 90, and then culturing said producer cell whereby said producer cell produces adeno-associated virus, preferably further comprising harvesting said adeno-associated virus, where said harvested adeno-associated virus comprises a transgene.

93. The method of claim 92, where the producer cell produces
a) virus preparation wherein the ratio of the number of capsids containing the gene or genome of interest to the number of total physical capsids is at least as high as the ratio of the number of capsids containing the gene or genome of interest to the number of total physical capsids produced by a similar cell containing a wild-type adeno-associated virus genome:
b) virus having a ratio of full: empty virus capsids least as high as does a similar cell infected with a wild-type adeno-associated virus genome;
c) virus having a ratio of full: empty virus capsids 30% higher than does a similar cell infected with wild-type adeno-associated virus;
d) virus having at least as many viral genomes/mL as does a similar cell infected with wild-type adeno-associated virus; or
e) having at least four times as many viral genomes/mL as does a similar cell infected with wild-type adeno-associated virus.

94. The method of claim 92, wherein the producer cell is cultured for at least 30 hours, at least 36 hours, at least 48 hours, at least 72 hours, at least 96 hours.

95. Adeno-associated virus produced by the method of claim 92.

96. A method of increasing stability, increasing capsid integrity, or reducing capsid degradation of an adeno-associated virus (AAV), comprising including in the AAV the adeno-associated virus genome of claim 83.

97. A method of increasing the proportion of AAV capsids containing a gene or genome of interest, comprising including in the AAV the adeno-associated virus genome of claim 83 and the gene or genome of interest.

98. A method of the increasing the viral titre (viral genomes/mL) of a producer cell producing an AAV, comprising including in the AAV the adeno-associated virus genome of claim 83 and introducing the AAV in the producer cell.

99. A method for increasing the retention of viral genomes or viral particles in a producer cell producing an AAV, comprising including in the AAV the adeno-associated virus genome of claim 83 and introducing the AAV in the producer cell, preferably, further comprising harvesting and/or purifying the viral genomes or viral particles from the producer cells, preferably substantially free of media.


SEQUENCE LISTING (48 sequences)

SEQ ID NO: 1 - protein, 120 aa, Artificial Sequence
  AAV1 MAAP
NLEPRSPKPTSKSRTTAGVWCFLATSTSDPSTDSTRGSPSTRRTQRPSSTTPRTTSSSKRVTIRTCGITTPTPSFRSVCKKIRLLGATSGEQSSRPRSGFSNLSVWLRKALRRLLERNVR

SEQ ID NO: 2 - protein, 119 aa, Artificial Sequence
  AAV2 MAAP
LAHHHQSPQSGIRTTAGVLCFLGTSTSDPSTDSTRESRSTRQTPRPSSTTKPTTGSSTAETTRTSSTTTPTRSFRSALKKIRLLGATSDEQSSRRKRGFLNLWAWLRNLLRRLREKRGR

SEQ ID NO: 3 - protein, 120 aa, Artificial Sequence
  AAV3 MAAP
NLESLNPKRTNNTRTTVGVLCFRVTNTSDPVTDSTKESRSTRRTRQPSNTTKLTTSSSRPVTTRTSSTTTPTPSFRSVFKKIRLLGATLAEQSSRPKRGSLSLLVWLRKQLKRLLERRGL

SEQ ID NO: 4 - protein, 134 aa, Artificial Sequence
  AAV4 MAAP
RTTSLKAFESGGRCNLEPLNPRQINNIRTTLGVLCFRVTNTSDPATDSTRGNPSTQRTRQPSSTTRPTTSSSRPVTTPTSSTTTPTRSSSSGFRATHRLGATSAEQSSRPKRGFLNLLVWLSKRVRRLLERRDR

SEQ ID NO: 5 - protein, 144 aa, Artificial Sequence
  AAV5 MAAP
SCLLLITLQIGWKKLVKVFASFWALKRAHRNQNPISSIKIKPVVLCCLVITISDPETVSIEESLSTGQTRSRESTTSRTTSSLRRETTPTSSTTTRTPSFRRSSPTTHPSGETSERQSFRPRKGFSNLLAWLKRVLRRPLPESG

SEQ ID NO: 6 - protein, 170 aa, Artificial Sequence
  AAV6 MAAP
NLEPRNPKPTSKSRTTAGVWCFLATSTSDPSTDSTRGSPSTRRMQRPSSTTRPTTSSSKRVTIRTCGITTPTPSFSTSDPSTDSTRGSPSTRRMQRPSSTTRPTTSSSKRVTIRTCGITTPTPSFRSVCKKIRLLGATSGEQSSRPRRGFSNLLVWLRKVLRRLLERNVR

SEQ ID NO: 7 - protein, 120 aa, Artificial Sequence
  AAV7 MAAP
NLEPRNPKPTSKSRTTAGVWCFLATSTSDPSTDSTRGSPSTRRTQRPSSTTRPTTSSSKRVTIRTCGITTPTPSFRSVCKKIRHLGATSGEQSSRPRSGFSNLSVWLRKALRRLLQRRDR

SEQ ID NO: 8 - protein, 120 aa, Artificial Sequence
  AAV8 MAAP
NLEPRSPKPTSKSRTTAGVWCFLATSTSDPSTDSTRGSPSTRRTQRPSSTTRPTTSSCRRVTIRTCGITTPTPSFRSVCKKIRLLGATSGEQSSRPRSGFSNLSVWLRKALRRLLERRDR

SEQ ID NO: 9 - protein, 119 aa, Artificial Sequence
  AAV9 MAAP
LEPLNPRQINNIKTTLEVLCFRVTNTLDPATDSTRGSRSTQQTRRPSSTTRPTTSSSRPETTRTSSTTTPTPSSRSGSKKIRLLGATSGEQSSRPKRGFLNLLVWLRKRLRRLLERRGL

SEQ ID NO: 10 - protein, 120 aa, Artificial Sequence
  AAV10 MAAP
NLEPPSPRPTSRSRTTAGVWCFLATSTSDPSTDSTRGSPSTRRTQRPSSTTRPTTSSSKRVTIRTCGITTPTPSFRSVCKKIRLLGATSGEQSSRPRSGFSNLSVWLRKLLRRLLERRDR

SEQ ID NO: 11 - protein, 120 aa, Artificial Sequence
  AAV consensus MAAP
  misc_feature (5)..(5): Xaa can be any naturally occurring amino acid
  misc_feature (8)..(8): Xaa can be any naturally occurring amino acid
  misc_feature (10)..(10): Xaa can be any naturally occurring amino acid
  misc_feature (12)..(12): Xaa can be any naturally occurring amino acid
  misc_feature (24)..(24): Xaa can be any naturally occurring amino acid
  misc_feature (39)..(39): Xaa can be any naturally occurring amino acid
  misc_feature (45)..(45): Xaa can be any naturally occurring amino acid
  misc_feature (78)..(79): Xaa can be any naturally occurring amino acid
  misc_feature (90)..(90): Xaa can be any naturally occurring amino acid
  misc_feature (110)..(110): Xaa can be any naturally occurring amino acid
NLEPXNPXPXSXIRTTAGVLCFLXTSTSDPSTDSTRGSXSTRRTXRPSSTTRPTTSSSRRVTTRTSSTTTPTPSFRSXXKKIRLLGATSXEQSSRPRRGFSNLLVWLRKXLRRLLERRGR

SEQ ID NO: 12 - DNA, 6952 bp, Artificial Sequence
  p0230-pUC-K-AAV2wt-Reverse
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccctgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcaggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaatttaattaatcagatccctcaatagcggccttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcacgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcatggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccacccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcatcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcaccttaaagaagatacatcttttgggggcaacctcagacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcacaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaattgcttgttaatcaataaaccgtttaattcgtttcagttgaactttggtctctgcgtatttctttcttatctagtttccatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaagccactcgagcacctaggaggcgcgccacggccgtagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 13 - DNA, 360 bp, Artificial Sequence
  AAV2 MAAP gene
ctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtag

SEQ ID NO: 14 - DNA, 6952 bp, Artificial Sequence
  p0189-AAV2WT-DS 540-546-554 -> stops
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccctgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcagagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcacgttggccgattcattaatgcagctggcacgacaggtttcccgactggaaagcgggcagtgagcgcaacgcaatttaattaatcagatccctcaatagcggccttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgtattagaggtcacgtgagtgttttgcgacattttgcgacaccatgtggtcacgctgggtatttaagcccaagtgagcacacagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcacggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcatggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactagacaagggagagccggtgaacgaggcagacgccgcggccctagagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaattgcttgttaatcaataaaccgtttaattcgtttcagttgaactttggtctctgcgtatttctttcttatctagtttccatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaagccactcgagcacctaggaggcgcgccacggccgtagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 15 - protein, 735 aa, Artificial Sequence [sequence omitted]

SEQ ID NO: 16 - protein, 32 aa, Artificial Sequence
  p0189-AAV2WT MAAP #1
LAHHHQSPQSGIRTTAGVLCFLGTSTSDPSTD

SEQ ID NO: 17 - protein, 37 aa, Artificial Sequence
  p0189-AAV2WT MAAP #2
LAHHHQSPQSGIRTTAGVLCFLGTSTSDPSTDTRESR

SEQ ID NO: 18 - protein, 44 aa, Artificial Sequence
  p0189-AAV2WT MAAP #3
LAHHHQSPQSGIRTTAGVLCFLGTSTSDPSTDTRESRTRQTPRP

SEQ ID NO: 19 - DNA, 24 bp, Artificial Sequence
  Rep2-PRB
  misc_feature (1)..(2): ZEN
  misc_feature (1)..(1): 56-FAM is before residue 1.
  misc_feature (2)..(2): 3IABkFQ is after residue 2.
cccgtgtcagaatctcaacccgtt

SEQ ID NO: 20 - DNA, 21 bp, Artificial Sequence
  Rep2-FWD
cttcactcacggacagaaaga

SEQ ID NO: 21 - DNA, 20 bp, Artificial Sequence
  Rep2-Rev
ctggcacctttcccatgata

SEQ ID NO: 22 - DNA, 24 bp, Artificial Sequence
  Ad5-E4-PRB
  misc_feature (1)..(1): 56-FAM is before residue 1.
  misc_feature (1)..(2): ZEN
  misc_feature (2)..(2): 3IABkFQ is after residue 2.
acccagccaacctacacattcgtt

SEQ ID NO: 23 - DNA, 20 bp, Artificial Sequence
  Ad5-E4-FWD
catccaccaccgcagaataa

SEQ ID NO: 24 - DNA, 21 bp, Artificial Sequence
  Ad5-E4-REV
acatggttcttccagctcttc

SEQ ID NO: 25 - DNA, 24 bp, Artificial Sequence
  Kan-PRB
  misc_feature (1)..(1): 56-FAM is before residue 1.
  misc_feature (1)..(2): ZEN
  misc_feature (2)..(2): 3IABkFQ is after residue 2.
tcgcacctgattgcccgacattat

SEQ ID NO: 26 - DNA, 20 bp, Artificial Sequence
  Kan-FWD
atcgggcttcccatacaatc

SEQ ID NO: 27 - DNA, 19 bp, Artificial Sequence
  Kan-REV
gctctaggccgcgattaaa

SEQ ID NO: 28 - protein, 20 aa, Artificial Sequence
  anti-MAAP antigen
KKIRLLGATSDEQSSRRKRG

SEQ ID NO: 29 - protein, 19 aa, Artificial Sequence
  Anti-MAAP antigen
RSTSSRTSSARRIKDASRR

SEQ ID NO: 30 - protein, 4 aa, Artificial Sequence
  basic-amino-acid-rich (BR) cluster
KKIR

SEQ ID NO: 31 - protein, 4 aa, Artificial Sequence
  basic-amino-acid-rich (BR) cluster
RRKR

SEQ ID NO: 32 - protein, 13 aa, Artificial Sequence
  basic-amino-acid-rich (BR) cluster
RNLLRRLREKRGR

SEQ ID NO: 33 - DNA, 22 bp, Artificial Sequence
  primer CMV-Fwd
catgaccttatgggactttcct

SEQ ID NO: 34 - DNA, 21 bp, Artificial Sequence
  primer CMV-Rev
ctatccacgcccattgatgta

SEQ ID NO: 35 - DNA, 2211 bp, adeno-associated virus serotype 1
atggctgccgatggttatcttccagattggctcgaggacaacctctctgagggcattcgcgagtggtgggacttgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaaacgtccggtagagcagtcgccacaagagccagactcctcctcgggcatcggcaagacaggccagcagcccgctaaaaagagactcaattttggtcagactggcgactcagagtcagtccccgatccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgatttcaacagattccactgccacttttcaccacgtgactggcagcgactcatcaacaacaattggggattccggcccaagagactcaacttcaaactcttcaacatccaagtcaaggaggtcacgacgaatgatggcgtcacaaccatcgctaataaccttaccagcacggttcaagtcttctcggactcggagtaccagcttccgtacgtcctcggctctgcgcaccagggctgcctccctccgttcccggcggacgtgttcatgattccgcaatacggctacctgacgctcaacaatggcagccaagccgtgggacgttcatccttttactgcctggaatatttcccttctcagatgctgagaacgggcaacaactttaccttcagctacacctttgaggaagtgcctttccacagcagctacgcgcacagccagagcctggaccggctgatgaatcctctcatcgaccaatacctgtattacctgaacagaactcaaaatcagtccggaagtgcccaaaacaaggacttgctgtttagccgtgggtctccagctggcatgtctgttcagcccaaaaactggctacctggaccctgttatcggcagcagcgcgtttctaaaacaaaaacagacaacaacaacagcaattttacctggactggtgcttcaaaatataacctcaatgggcgtgaatccatcatcaaccctggcactgctatggcctcacacaaagacgacgaagacaagttctttcccatgagcggtgtcatgatttttggaaaagagagcgccggagcttcaaacactgcattggacaatgtcatgattacagacgaagaggaaattaaagccactaaccctgtggccaccgaaagatttgggaccgtggcagtcaatttccagagcagcagcacagaccctgcgaccggagatgtgcatgctatgggagcattacctggcatggtgtggcaagatagagacgtgtacctgcagggtcccatttgggccaaaattcctcacacagatggacactttcacccgtctcctcttatgggcggctttggactcaagaacccgcctcctcagatcctcatcaaaaacacgcctgttcctgcgaatcctccggcggagttttcagctacaaagtttgcttcattcatcacccaatactccacaggacaagtgagtgtggaaattgaatgggagctgcagaaagaaaacagcaagcgctggaatcccgaagtgcagtacacatccaattatgcaaaatctgccaacgttgattttactgtggacaacaatggactttatactgagcctcgccccattggcacccgttaccttacccgtcccctgtaa

SEQ ID NO: 36 - DNA, 2208 bp, adeno-associated virus serotype 2
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 37 - DNA, 2211 bp, adeno-associated virus serotype 3
atggctgctgacggttatcttccagattggctcgaggacaacctttctgaaggcattcgtgagtggtgggctctgaaacctggagtccctcaacccaaagcgaaccaacaacaccaggacaaccgtcggggtcttgtgcttccgggttacaaatacctcggacccggtaacggactcgacaaaggagagccggtcaacgaggcggacgcggcagccctcgaacacgacaaagcttacgaccagcagctcaaggccggtgacaacccgtacctcaagtacaaccacgccgacgccgagtttcaggagcgtcttcaagaagatacgtcttttgggggcaaccttggcagagcagtcttccaggccaaaaagaggatccttgagcctcttggtctggttgaggaagcagctaaaacggctcctggaaagaagggggctgtagatcagtctcctcaggaaccggactcatcatctggtgttggcaaatcgggcaaacagcctgccagaaaaagactaaatttcggtcagactggagactcagagtcagtcccagaccctcaacctctcggagaaccaccagcagcccccacaagtttgggatctaatacaatggcttcaggcggtggcgcaccaatggcagacaataacgagggtgccgatggagtgggtaattcctcaggaaattggcattgcgattcccaatggctgggcgacagagtcatcaccaccagcaccagaacctgggccctgcccacttacaacaaccatctctacaagcaaatctccagccaatcaggagcttcaaacgacaaccactactttggctacagcaccccttgggggtattttgactttaacagattccactgccacttctcaccacgtgactggcagcgactcattaacaacaactggggattccggcccaagaaactcagcttcaagctcttcaacatccaagttagaggggtcacgcagaacgatggcacgacgactattgccaataaccttaccagcacggttcaagtgtttacggactcggagtatcagctcccgtacgtgctcgggtcggcgcaccaaggctgtctcccgccgtttccagcggacgtcttcatggtccctcagtatggatacctcaccctgaacaacggaagtcaagcggtgggacgctcatccttttactgcctggagtacttcccttcgcagatgctaaggactggaaataacttccaattcagctataccttcgaggatgtaccttttcacagcagctacgctcacagccagagtttggatcgcttgatgaatcctcttattgatcagtatctgtactacctgaacagaacgcaaggaacaacctctggaacaaccaaccaatcacggctgctttttagccaggctgggcctcagtctatgtctttgcaggccagaaattggctacctgggccctgctaccggcaacagagactttcaaagactgctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactggaactgtcaatcatcagggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcatgatcaaaaatactccggtaccggcaaatcctccgacgactttcagcccggccaagtttgcttcatttatcactcagtactccactggacaggtcagcgtggaaattgagtgggagctacagaaagaaaacagcaaacgttggaatccagagattcagtacacttccaactacaacaagtctgttaatgtggactttactgtagacactaatggtgtttatagtgaacctcgccctattggaacccggtatctcacacgaaacttgtga

SEQ ID NO: 38 - DNA, 2205 bp, adeno-associated virus serotype 4
atgactgacggttaccttccagattggctagaggacaacctctctgaaggcgttcgagagtggtgggcgctgcaacctggagcccctaaacccaaggcaaatcaacaacatcaggacaacgctcggggtcttgtgcttccgggttacaaatacctcggacccggcaacggactcgacaagggggaacccgtcaacgcagcggacgcggcagccctcgagcacgacaaggcctacgaccagcagctcaaggccggtgacaacccctacctcaagtacaaccacgccgacgcggagttccagcagcggcttcagggcgacacatcgtttgggggcaacctcggcagagcagtcttccaggccaaaaagagggttcttgaacctcttggtctggttgagcaagcgggtgagacggctcctggaaagaagagaccgttgattgaatccccccagcagcccgactcctccacgggtatcggcaaaaaaggcaagcagccggctaaaaagaagctcgttttcgaagacgaaactggagcaggcgacggaccccctgagggatcaacttccggagccatgtctgatgacagtgagatgcgtgcagcagctggcggagctgcagtcgagggcggacaaggtgccgatggagtgggtaatgcctcgggtgattggcattgcgattccacctggtctgagggccacgtcacgaccaccagcaccagaacctgggtcttgcccacctacaacaaccacctctacaagcgactcggagagagcctgcagtccaacacctacaacggattctccaccccctggggatactttgacttcaaccgcttccactgccacttctcaccacgtgactggcagcgactcatcaacaacaactggggcatgcgacccaaagccatgcgggtcaaaatcttcaacatccaggtcaaggaggtcacgacgtcgaacggcgagacaacggtggctaataaccttaccagcacggttcagatctttgcggactcgtcgtacgaactgccgtacgtgatggatgcgggtcaagagggcagcctgcctccttttcccaacgacgtctttatggtgccccagtacggctactgtggactggtgaccggcaacacttcgcagcaacagactgacagaaatgccttctactgcctggagtactttccttcgcagatgctgcggactggcaacaactttgaaattacgtacagttttgagaaggtgcctttccactcgatgtacgcgcacagccagagcctggaccggctgatgaaccctctcatcgaccagtacctgtggggactgcaatcgaccaccaccggaaccaccctgaatgccgggactgccaccaccaactttaccaagctgcggcctaccaacttttccaactttaaaaagaactggctgcccgggccttcaatcaagcagcagggcttctcaaagactgccaatcaaaactacaagatccctgccaccgggtcagacagtctcatcaaatacgagacgcacagcactctggacggaagatggagtgccctgacccccggacctccaatggccacggctggacctgcggacagcaagttcagcaacagccagctcatctttgcggggcctaaacagaacggcaacacggccaccgtacccgggactctgatcttcacctctgaggaggagctggcagccaccaacgccaccgatacggacatgtggggcaacctacctggcggtgaccagagcaacagcaacctgccgaccgtggacagactgacagccttgggagccgtgcctggaatggtctggcaaaacagagacatttactaccagggtcccatttgggccaagattcctcataccgatggacactttcacccctcaccgctgattggtgggtttgggctgaaacacccgcctcctcaaatttttatcaagaacaccccggtacctgcgaatcctgcaacgaccttcagctctactccggtaaactccttcattactcagtacagcactggccaggtgtcggtgcagattgactgggagatccagaaggagcggtccaaacgctggaaccccgaggtccagtttacctccaactacggacagcaaaactctctgttgtgggctcccgatgcggctgggaaatacactgagcctagggctatcggtacccgctacctcacccaccacctgtaa

SEQ ID NO: 39 - DNA, 2175 bp, adeno-associated virus serotype 5
atgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaa

SEQ ID NO: 40 - DNA, 2211 bp, adeno-associated virus serotype 6
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggttctcgaaccttttggtctggttgaggaaggtgctaagacggctcctggaaagaaacgtccggtagagcagtcgccacaagagccagactcctcctcgggcattggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtccggaagtgcccaaaacaaggacttgctgtttagccgggggtctccagctggcatgtctgttcagcccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaa

SEQ ID NO: 41 - DNA, 2214 bp, adeno-associated virus serotype 7
atggctgccgatggttatcttccagattggctcgaggacaacctctctgagggcattcgcgagtggtgggacctgaaacctggagccccgaaacccaaagccaaccagcaaaagcaggacaacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcatttgggggcaacctcgggcgagcagtcttccaggccaagaagcgggttctcgaacctctcggtctggttgaggaaggcgctaagacggctcctgcaaagaagagaccggtagagccgtcacctcagcgttcccccgactcctccacgggcatcggcaagaaaggccagcagcccgccagaaagagactcaatttcggtcagactggcgactcagagtcagtccccgaccctcaacctctcggagaacctccagcagcgccctctagtgtgggatctggtacagtggctgcaggcggtggcgcaccaatggcagacaataacgaaggtgccgacggagtgggtaatgcctcaggaaattggcattgcgattccacatggctgggcgacagagtcattaccaccagcacccgaacctgggccctgcccacctacaacaaccacctctacaagcaaatctccagtgaaactgcaggtagtaccaacgacaacacctacttcggctacagcaccccctgggggtattttgactttaacagattccactgccacttctcaccacgtgactggcagcgactcatcaacaacaactggggattccggcccaagaagctgcggttcaagctcttcaacatccaggtcaaggaggtcacgacgaatgacggcgttacgaccatcgctaataaccttaccagcacgattcaggtattctcggactcggaataccagctgccgtacgtcctcggctctgcgcaccagggctgcctgcctccgttcccggcggacgtcttcatgattcctcagtacggctacctgactctcaacaatggcagtcagtctgtgggacgttcctccttctactgcctggagtacttcccctctcagatgctgagaacgggcaacaactttgagttcagctacagcttcgaggacgtgcctttccacagcagctacgcacacagccagagcctggaccggctgatgaatcccctcatcgaccagtacttgtactacctggccagaacacagagtaacccaggaggcacagctggcaatcgggaactgcagttttaccagggcgggccttcaactatggccgaacaagccaagaattggttacctggaccttgcttccggcaacaaagagtctccaaaacgctggatcaaaacaacaacagcaactttgcttggactggtgccaccaaatatcacctgaacggcagaaactcgttggttaatcccggcgtcgccatggcaactcacaaggacgacgaggaccgctttttcccatccagcggagtcctgatttttggaaaaactggagcaactaacaaaactacattggaaaatgtgttaatgacaaatgaagaagaaattcgtcctactaatcctgtagccacggaagaatacgggatagtcagcagcaacttacaagcggctaatactgcagcccagacacaagttgtcaacaaccagggagccttacctggcatggtctggcagaaccgggacgtgtacctgcagggtcccatctgggccaagattcctcacacggatggcaactttcacccgtctcctttgatgggcggctttggacttaaacatccgcctcctcagatcctgatcaagaacactcccgttcccgctaatcctccggaggtgtttactcctgccaagtttgcttcgttcatcacacagtacagcaccggacaagtcagcgtggaaatcgagtgggagctgcagaaggaaaacagcaagcgctggaacccggagattcagtacacctccaactttgaaaagcagactggtgtggactttgccgttgacagccagggtgtttactctgagcctcgccctattggcactcgttacctcacccgtaatctgtaa

SEQ ID NO: 42 - DNA, 2217 bp, adeno-associated virus serotype 8
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 43 - DNA, 2211 bp, adeno-associated virus serotype 9
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 44 - DNA, 2217 bp, adeno-associated virus serotype 10
atggctgctgacggttatcttccagattggctcgaggacaacctctctgagggcattcgcgagtggtgggacctgaaacctggagcccccaagcccaaggccaaccagcagaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaagctgctaagacggctcctggaaagaagagaccggtagaaccgtcacctcagcgttcccccgactcctccacgggcatcggcaagaaaggccagcagcccgctaaaaagagactgaactttgggcagactggcgagtcagagtcagtccccgaccctcaaccaatcggagaaccaccagcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgaacctgggccctgcccacctacaacaaccacctctacaagcaaatctccaacgggacatcgggaggaagcaccaacgacaacacctacttcggctacagcaccccctgggggtattttgacttcaacagattccactgccacttctcaccacgtgactggcagcgactcatcaacaacaactggggattccggccaaaaagactcagcttcaagctcttcaacatccaggtcaaggaggtcacgcagaatgaaggcaccaagaccatcgccaataaccttaccagcacgattcaggtatttacggactcggaataccagctgccgtacgtcctcggctccgcgcaccagggctgcctgcctccgttcccggcggatgtcttcatgattccccagtacggctacctgacactgaacaatggaagtcaagccgtaggccgttcctccttctactgcctggaatattttccatctcaaatgctgcgaactggaaacaattttgaattcagctacaccttcgaggacgtgcctttccacagcagctacgcacacagccagagcttggaccgactgatgaatcctctcattgaccagtacctgtactacttatccagaactcagtccacaggaggaactcaaggtacccagcaattgttattttctcaagctgggcctgcaaacatgtcggctcaggccaagaactggctgcctggaccttgctaccggcagcagcgagtctccacgacactgtcgcaaaacaacaacagcaactttgcttggactggtgccaccaaatatcacctgaacggaagagactctctggtgaatcccggtgtcgccatggcaacccacaaggacgacgaggaacgcttcttcccgtcgagcggagtcctgatgtttggaaaacagggtgctggaagagacaatgtggactacagcagcgttatgctaacaagcgaagaagaaattaaaaccactaaccctgtagccacagaacaatacggcgtggtggctgacaacttgcagcaagccaatacagggcctattgtgggaaatgtcaacagccaaggagccttacctggcatggtctggcagaaccgagacgtgtacctgcagggtcccatctgggccaagattcctcacacggacggcaactttcacccgtctcctctgatgggcggctttggacttaaacacccgcctccacagatcctgatcaagaacacgccggtacctgcggatcctccaacaacgttcagccaggcgaaattggcttccttcatcacgcagtacagcaccggacaggtcagcgtggaaatcgagtgggagctgcagaaggagaacagcaaacgctggaacccagagattcagtacacttcaaactactacaaatctacaaatgtggactttgctgtcaatacagagggaacttattctgagcctcgccccattggtactcgttatctgacacgtaatctgtaa

SEQ ID NO: 45 - DNA, 24 bp, Artificial Sequence
  Rep2-PRB
  misc_feature (1)..(1): 56-FAM
  misc_feature (9)..(9): ZEN
  misc_feature (24)..(24): 3IABkFQ
cccgtgtcagaatctcaacccgtt

SEQ ID NO: 46 - DNA, 24 bp, Artificial Sequence
  Ad5-E4-PRB
  misc_feature (1)..(1): 56-FAM
  misc_feature (9)..(9): ZEN
  misc_feature (24)..(24): 3IABkFQ
acccagccaacctacacattcgtt

SEQ ID NO: 47 - DNA, 24 bp, Artificial Sequence
  Kan-PRB
  misc_feature (1)..(1): 56-FAM
  misc_feature (9)..(9): ZEN
  misc_feature (24)..(24): 3IABkFQ
tcgcacctgattgcccgacattat

SEQ ID NO: 48 - DNA, 24 bp, Artificial Sequence
  CMV-PRB
  misc_feature (1)..(1): 6-FAM
  misc_feature (9)..(9): ZEN
  misc_feature (24)..(24): IABkFQ
tcgcacctgattgcccgacattat